(19) United States
(12) Patent Application Publication
(10) Pub. No.: US 2023/0053864 A1
(43) Pub. Date: Feb. 23, 2023

(54) Methods for Identifying Modulators of G Protein-Coupled Receptors

(71) Applicant: University of Miami, Miami, FL (US)

(72) Inventors: Daniel Isom, Miami, FL (US); William Morgan, Miaimi, FL (US); Nicholas Kapolka, Miami, FL (US); Geoffrey Taghon, Miami, FL (US); Jacob Rowe, Miami, FL (US)

(21) Appl. No.: 17/785,524

(22) PCT Filed: Dec. 16, 2020
(86) PCT No.: PCT/US2020/065247

§ 371 Date: Jun. 15, 2022

Related U.S. Application Data
(60) Provisional application No. 62/949,069, filed on Dec. 17, 2019.
(60) Provisional application No. 62/985,960, filed on Mar. 6, 2020.

(51) Int. Cl. G01N 33/68; C07K 14/705; C12N 15/90

(52) U.S. Cl. CPC G01N 33/6872; C07K 14/705; C12N 15/905; C12N 2310/20; G01N 2500/04; G01N 2500/10

(57) ABSTRACT

The disclosure relates to a plurality of cells, compositions and methods for identifying modulators of a target protein. The cells, compositions and methods comprise a (i) a target domain gene (ii) an intracellular chimeric G-protein alpha subunit comprising an endogenous G-protein alpha subunit with a humanized C-terminus; and (iii) an inducible reporter, wherein the expression of the reporter is dependent on the activation of the target domain encoded by target domain gene, and wherein the target domain gene comprises a barcode. The disclosure further relates to a host cell comprising a plurality of exogenous landing pads integrated in the host cell's genome, wherein each exogenous landing pad is integrated at a safe harbor genome loci in the host cell's genome.

### CROSS-REFERENCE TO RELATED APPLICATIONS

This application claims the benefit of U.S. Provisional Patent Application Ser. No. 62/985,960 filed on Mar. 6, 2020 and U.S. Provisional Patent Application Ser. No. 62/949,069 filed on Dec. 17, 2019, the disclosures of each of which are explicitly incorporated by reference herein.

### STATEMENT REGARDING FEDERALLY FUNDED RESEARCH OR DEVELOPMENT

This invention was made with government support under GM119518 and TR0029086 awarded by the National Institutes of Health. The government has certain rights in the invention.

### FIELD OF THE DISCLOSURE

The disclosure relates to a plurality of cells, compositions and methods for identifying modulators of a target protein. The cells, compositions and methods comprise a (i) one or more of a target domain gene that specifically binds to a binding partner (ii) one or more of an intracellular chimeric G-protein alpha subunit comprising an endogenous G-protein alpha subunit with a humanized C-terminus; and (iii) one or more of an inducible reporter, wherein the expression of the reporter is dependent on the activation of the target domain encoded by target domain gene, and wherein the target domain gene comprises a barcode. The disclosure further relates to a host cell comprising a plurality of exogenous expression cassettes, herein after referred to as landing pads, integrated in the host cell's genome, wherein each exogenous landing pad is integrated at a safe harbor genome loci in the host cell's genome.

### BACKGROUND

Metabolites function both as energy sources and biosynthetic building blocks. However, many metabolites from bacteria (e.g. short-chain fatty acid and bile acid metabolites) and humans (e.g. lactate, succinate, ketone bodies) are known to function as extracellular signaling molecules similar to neurotransmitters and hormones (Husted et al., (2017) Cell Metab 25, 777-796). G protein-coupled receptors (GPCRs) are the largest and most diverse group of membrane receptors in humans (Fredriksson et al., (2003) Mol Pharmacol 63, 1256-1272). Having over 800 members, with more than 360 detecting endogenous ligands (endoGPCRs) (Alexander et al., (2017) TBr J Pharmacol 174 Suppl 1, S17-S129), it is likely that many of the 114,000 known dietary, bacterial, and metabolically-derived human metabolites (Wishart et al., (2018) Nucleic Acids Res 46, D608-D617) target numerous endoGPCRs. However, only a small subset of endoGPCRs (-20) residing primarily in the enteroendocrine, neuronal, and immune cells of the gut and liver are currently classified as metabolite sensors (mGPCRs) (Husted et al., (2017) Cell Metab 25, 777-796). It is likely that many more mGPCRs remain undiscovered throughout the body where they detect local autocrine and paracrine metabolite signals with nanomolar to micromolar affinity.

GPCRs mediate cellular decision-making and physiological processes by detecting a wide variety of chemical signals, such as small molecule regulators, peptides, and proteins. GPCRs transduce these extracellular signals across the plasma membrane to activate intracellular G proteins that amplify the receptor response through a variety of downstream second messengers (cAMP, IP3, DAG, and Ca²⁺). While more than 360 endoGPCRs comprise the largest and most therapeutically targeted class of cell surface receptors in humans, only 30-40% have well-defined biological ligands and are currently druggable (Sriram and Insel (2018) Mol Pharmacol 93, 251-258). The remaining 60-70%, many of which are classified as “pharmacologically dark” by the Illuminating the Druggable Genome program initiated by the National Institutes of Health (Rodgers et al., (2018) Nat Rev Drug Discov 17, 301-302), represent a substantial opportunity to advance biological insights that improve understanding of metabolic (dys)regulation and inform drug development.

Thus there is a need in this art for reagents and methods for identifying metabolic or other ligands involved in the activity of the many uncharacterized GPCRs in humans and other organisms.

SUMMARY

Provided herein is a plurality of cells, wherein each cell comprises (i) one or more of a target domain gene that specifically binds to a binding partner (ii) one or more of an intracellular chimeric G-protein alpha subunit comprising an endogenous G-protein alpha subunit with a humanized C-terminus; and (iii) one or more of an inducible reporter, wherein the expression of the reporter is dependent on the activation of the target domain encoded by the target domain gene, and wherein the target domain gene comprises a barcode.

Also provided herein are methods for identifying a compound capable of modulating the activity of a target domain, comprising: (a) contacting the plurality of cells of with a compound; (b) determining the activity of the target domain by detecting the reporter; wherein detection of the reporter in the cell indicates that the compound interacts with the target domain.

Also provided herein is a yeast cell comprising a plurality of exogenous landing pads integrated in the yeast cell's genome, wherein each exogenous landing pad is integrated at a safe harbor genome loci in the yeast cell's genome.

### BRIEF DESCRIPTION OF THE DRAWINGS

FIGS. 1(A)-(E) illustrate the engineering and validation of the dCyFIRscreen profiling method. FIG. 1(A) shows a simplified schematic of the yeast pheromone pathway model for studying human GPCRs (see FIG. 2 for further details). FIG. 1(B) shows the workflow of the high-throughput CRISPR/Cas9 genome editing pipeline showing primary screening data (more than 5,000 mTq2 fluorescence measurements) for the exploratory panel of 30 human GPCRs. All fluorescence values are reported as relative fluorescence units (RFUs). FIG. 1(C) shows dCyFIRscreen profiles for 300 GPCR-Gα strains against their known agonists, with error bars representing the SD of n=4 technical replicates. Untreated/treated conditions are represented by white/colored bars. All RFU measurements were quantified using the same gain setting. FIG. 1(D) shows heat map of agonist-induced signaling in the 300 GPCR-Gα strains. FIG. 1(E) shows heat map of constitutive activity in the 300 GPCR-Gα strains.

FIGS. 2(A)-(E) illustrate engineering and characterization of the base GPCR-Gα reporter strains. FIG. 2(A) shows the schematic of the engineered yeast pheromone pathway for studying human GPCRs. A gene encoding a human GPCR was directly integrated into the yeast genome into a synthetic expression cassette at the X-2 locus. Human GPCR signaling through a chimeric yeast Gα protein was prolonged due to the deletion of a negative regulator (RGS protein, SST2). MAP kinase cascade signaling drives the expression of a bright cyan fluorescent protein, mTq2, that was directly installed into the yeast genome replacing a pheromone-responsive gene, FIG. 1. Deletion of FAR1 prevents cell cycle arrest upon MAPK signaling. FIG. 2(B) shows the 16 genes encoding human Gα proteins can be represented by 10 degenerate Gα chimeras (10 unique yeast strains), each with the corresponding 5 C-terminal amino acids from humans. FIG. 2(C) shows the dose-response curves to the yeast pheromone, a-factor, for negative (strain lacking mTq2) and positive (strain overexpressing mTq2 from the X-2 expression cassette) controls, and for strains with mTq2 replacing two pheromone-responsive genes (FIG. 1 and Fus1) with the endogenous yeast GPCR, Ste2, expressed from its endogenous locus. Error bars represent SD of n=3 technical replicates (n=1 for controls). Expression of mTq2 from the FIG. 1 locus was determined to be optimal due to the lower background signal. FIG. 2(D) shows the FACS analysis of the strains in panel (C) corroborates the results of the titrations in (C). FIG. 2(E) shows qPCR data illustrating similar heterotrimeric G protein expression levels across all 10 GPCR-Gα reporter strains. Data reported as Cq values normalized to two housekeeping genes (ALG9 and TAF10) with error bars representing SEM of n=3-5 technical replicates.

FIGS. 3(A)-(E) illustrate the developing and validation of the dCyFIRplex profiling method. FIG. 3(A) shows a schematic of the dCyFIRplex workflow showing strain consolidation to build the multiplex, FACS to collect active receptor strain pools, and the two primary multiplex deconvolution techniques. FIG. 3(B) shows confocal microscopy images of treated and untreated (vehicle) samples of the GPCR-Gα 300-plex with the added mRuby3 tracer strain (maximum intensity projections, 63× magnification). FIG. 3(C) shows FACS analysis of the inactive (gray), active (cyan), and tracer (red) pools for the 300-plex shown in panel B. FIG. 3(D) shows FACS analysis of negative (gray), positive (cyan), and tracer (red) controls; also, a representative standard curve of tracer event counts versus active pool event counts for our reference conditions of −/+adenosine used to calibrate the FACS sorting procedure. T racer event counts between 3-5 k gave the most consistent deconvolution results. FIG. 3(E) shows PCR deconvolution of SUCNR1 and HTR4 10-plexes and combined 20-plex visualized by gel electrophoresis; also, the family of normalized titration curves that corresponded to the active GPCR-Gα reporter strains in each 10- and 20-plex (errors bars represent the SD of n=4 technical replicates). FIG. 3(F) shows dCyFIRplex profiles deconvoluted via qPCR for the agonist-treated 300-plexes characterized in panels B and C. Expected hits are colored blue. ΔCq values correspond to the Cq difference between treated and untreated conditions, with error bars representing the SEM of n=6 repeats derived from 3 independent 300-plex consolidations deconvoluted in technical duplicate. ΔCq values correspond to a log₂ scale.

FIGS. 4(A)-(B) illustrate using dCyFIRplex profiling to recapitulate known agonist interactions. FIG. 4(A) shows dCyFIRplex profiles for known GPCR agonists in the 30-receptor panel deconvoluted via qPCR. FIG. 4(B) shows the same samples as in panel A deconvoluted using NanoString. FIGS. 4(A, B) show the ΔCq values correspond to the Cq difference between treated and untreated conditions, with error bars representing the SEM of n=6 repeats derived from 3 independent 300-plex consolidations deconvoluted in technical duplicate. ΔCq values correspond to a loge scale. NanoString transcript counts were collected in technical duplicate, averaged, and normalized to the maximal RNA transcript count for each GPCR gene.

FIGS. 5(A)-(D) illustrate using dCyFIRplex to discover new interactions for known GPCR metabolite agonists. FIG. 5(A) shows dCyFIRplex profiles identifying new GPCR-ligand interactions (pink bars) discovered in the process of screening known agonists (purple bars) within the panel of 30 exploratory receptors. FIG. 5(B) shows dCyFIRscreen profiles confirming the dCyFIRplex discoveries in panel A, with error bars representing the SD of n=4 technical replicates. FIG. 5(C and D) shows select titrations confirming the dCyFIRplex discoveries in panel A, with error bars representing the SD of n=4 technical replicates. Dashed lines and boxes indicate datasets showing that KYNA activates HCAR3 with greater potency than GPR35 and is also an endogenous negative allosteric modulator of ADRA2B.

FIGS. 6(A)-(F) illustrate the follow-up titrations and control experiments for new ligand discoveries related to FIG. 5. FIG. 6(A) shows the dose-response curves for S1PR1 and S1PR2 with lysophosphatidic acid (LPA). Error bars represent SD of n=4 technical replicates. FIG. 6(B) shows the dose-response curves for HCAR3 with 3-hydroxyoctanoic acid (left) and 3-hydroxyoctanoic acid (dashed line, open circles) in the presence of kynurenic acid (solid line, closed circles) (right). Error bars represent SD of n=4 technical replicates. FIG. 6(C) shows the dose-response curves for ADRA2B with kynurenic acid (left) and epinephrine (dashed line, open circles) in the presence of kynurenic acid (solid line, closed circles) (right). Error bars represent SD of n=4 technical replicates. FIG. 6(D) shows the dose-response curves for GPR35 with kynurenic acid. Error bars represent SD of n=4 technical replicates. FIG. 6(E) shows the coarse 4-point titrations of ADRA2B with phenethylamine (PEA). Error bars represent SD of n=4 technical replicates. FIG. 6(F) shows the coarse 4-point titrations of ADRA2B, GPR35, and HCAR3 with L-kynurenine. The increase in fluorescence as a function of increasing L-kynurenine is due to the autofluorescence of L-kynurenine. Error bars represent SD of n=4 technical replicates.

FIGS. 7(A)-(D) illustrate dCyFIRscreen and dCyFIRplex profiling of a human metabolite library. FIG. 7(A) shows the step-by-step workflow used to screen a library of 320 endogenous human metabolites. FIG. 7(B) shows the Z-score profiles for metabolite screens of receptor set 1 (ADORA1, ADORA2A, FFAR2, GPR4, GPR65, GPR68, HCAR2, HCAR3, LPAR1, LPAR4, MRGPRD), set 2 (ADORA2B, ADRA2A, ADRA2B, AVPR2, CHRM1, CHRM3, CHRM5, CNR2, GPR35), and set 3 (HTR4, MTNR1A, MTNR1B, PTAFR, PTGER3, S1PR1, S1PR2, S1PR3, SSTR5, SUCNR1). Grey bands indicate Z-scores between ±1. FIG. 7(C) shows fluorescence microscopy images for Z-score hits in receptor subsets 1 (110-plex), 2 (90-plex), and 3 (100-plex). FIG. 7(D) shows the discovery workflow illustrating tryptamine agonism of HTR4 and ADRA2B and dopamine agonism of ADRA2A and ADRA2B. Once tryptamine and dopamine were identified as hits (steps 1-3 in panel A), dCyFIRplex profiling was used to identify their GPCR target(s), dCyFIRscreen profiling to identify their Gα coupling pattern(s), and titrations to quantify their ECso values (steps 4-6 in panel A).

FIGS. 8(A)-(D) illustrate the identification, validation, and characterization of new GPCR-metabolite interactions. FIGS. 8(A, B) show dCyFIRplex profiles for (A) new metabolite agonists and (B) allosteric modulators, with error bars representing the SEM of n=6 repeats derived from 3 independent 300-plex consolidations deconvoluted in technical duplicate. FIGS. 8(C, D) show titrations of new metabolite (C) agonist and (D) positive allosteric modulator interactions in detailed (16-point) and coarse (4-point, FIG. 9) formats, with error bars representing the SD of n=4 technical replicates.

FIGS. 9(A)-(B) illustrate dCyFIRscreen profiles and coarse titrations used to confirm new metabolite agonists and allosteric modulators related to FIGS. 7 and 8. FIG. 9(A) shows the coarse 4-point titrations for ADRA2B with tryptamine and dopamine, and ADRA2A with dopamine. Error bars represent SD of n=4 technical replicates. FIG. 9(B) shows dCyFIRscreen profiles and coarse 4-point titrations for new metabolite interactions (2-amino-1-phenylethanol and petroselinic acid). Error bars represent SD of n=4 technical replicates.

FIGS. 10(A)-(B) illustrate detailed titrations for new metabolite allosteric modulators related to FIG. 8. FIG. 10(A) shows the dose-response curves for GPR65, ADORA2A, and GPR35 (dashed lines, open circles) in the presence of inositol (solid lines, closed circles). Error bars represent SD of n=4 technical replicates. FIG. 10(B) shows the dose-response curves for ADORA2A, GPR35, and HCAR3 (dashed lines, open circles) in the presence of DHEA (solid lines, closed circles). Error bars represent SD of n=4 technical replicates.

FIGS. 11(A)-(F) illustrate control dCyFIRscreen profiles for new metabolite allosteric modulators related to FIG. 8. FIG. 11(A) shows dCyFIRscreen profiles for GPCR-Gα reporter strains treated with inositol. Error bars represent SD of n=4 technical replicates. Blue boxes indicate PAM interactions. FIG. 11(B) shows dCyFIRscreen profiles for GPCR-Gα reporter strains treated with DHEA in EtOH. Error bars represent SD of n=4 technical replicates. Blue boxes indicate PAM interactions. FIG. 11(C) shows dCyFIRscreen profiles for GPCR-Gα reporter strains treated with DHEA in DMSO/EtOH. Error bars represent SD of n=4 technical replicates. Blue boxes indicate PAM interactions. FIG. 11(D) shows dCyFIRscreen profiles for GPCR-Gα reporter strains treated with androsterone. Error bars represent SD of n=4 technical replicates. Blue boxes indicate PAM interactions. FIG. 11(E) shows dCyFIRscreen profiles for GPCR-Gα reporter strains treated with DHEA-S. Error bars represent SD of n=4 technical replicates. FIG. 11(F) shows dCyFIRscreen profiles for GPCR-Gα reporter strains treated with cholesterol. Error bars represent SD of n=4 technical replicates.

FIG. 12 illustrates control dCyFIRscreen profiles used to assess dCyFIRplex sensitivity related to FIGS. 4-8. FIG. 12 shows the control dCyFIRscreen profiles for receptors with low ΔCq values from dCyFIRplex profiling. Error bars represent SD of n=4 technical replicates.

FIGS. 13(A)13(D) are schematics illustrating the process for humanizing the yeast pheromone pathway for GPCR studies. Simplified schematics of the (FIG. 13(A)) native and (FIG. 13 (B)) humanized pheromone pathway. In response to pheromone, the yeast GPCR Ste2 activates the intracellular Gα subunit to stimulate a MAP-kinase cascade that drives the expression of pheromone-responsive genes such as FIG1. In the humanized model, Ste2 is replaced with a human GPCR that is coupled to pheromone pathway via a Gα subunit chimera in which the last five residues of the native yeast Gα are replaced with the last five residues of a human Gα (indicated in orange). FIG. 13(C) shows that in the 2Δ reporter strain, the GTPase-activating protein SST2 is deleted, the cell cycle arrest factor FAR1 is deleted, and FIG1 is replaced with the transcriptional reporter mTq2. FIG. 13(D) shows the 3Δ reporter strain is derived from the 2Δreporter strain and has the additional deletion of the native yeast GPCR STE2.

FIGS. 14(A)-(B) illustrate installation and validation of individual CRISPR-addressable landing pads. FIG. 14A (Top) shows the variants of the 3Δ reporter strain, each containing a single landing pad placed at known safe harbor chromosome loci X-2, X-3, XI-2, or XII-5. Each landing pad contains a 20 bp Unique Targeting Sequence (UnTS) not found in the native yeast genome that provides a synthetic locus for CRISPR-addressable editing. FIG. 14A (Bottom) shows PCR validation of landing pad installation. PCR primers used were homologous to the native genomic loci sequences flanking the landing pads. Expected product sizes were 915 bp for X-2, X-3, and XII-5 landing pads and 816 bp for the XI-2 landing pad. FIG. 14 (B) shows rescuing pheromone signaling by expressing Ste2 from each landing pad in the single-padded 3Δ reporter strains. Data are reported as relative fluorescence units (RFU) at an OD₆₀₀ₘₙ of 1.0 and instrument gain 1200. Each strain was titrated with α-factor, the endogenous peptide pheromone for Ste2. Triangles correspond to strains expressing Ste2 from a landing pad: X-2 (turquoise), X-3 (red), XI-2 (purple), and XII-5 (green). Positive control titrations (labeled PC) correspond to the 2Δ reporter strain with Ste2 expressed from its native genome locus. Negative control titrations (labeled NC) correspond to the empty-padded 3Δ reporter strains described in panel A.

FIGS. 15(A)-(C) illustrate engineering four-padded strains for studying human GPCRs. FIG. 15(A) shows the engineered components of the 10 single-padded (left) and 10 four-padded (right) GPCR reporter strains used in this disclosure. Although the full set of 10 humanized Gα chimeras are shown, each of the 10 single-padded and 10 four-padded padded GPCR reporter strains contained only one Gα chimera. FIG. 15(B) shows validation of the functionality of each landing pad in the four-padded GPCR-Gα_{I} reporter strain using mTq2. The negative control, NC, corresponds to the empty four-padded GPCR-Gα_{I} reporter strain. FIG. 15(C) shows validation of the functionality of each landing pad in the GPCR reporter strains using constitutively active human GPR68. For FIGS. 15(B) and (C), data are reported as RFU at an OD₆₀₀ₘₙ of 1.0 and instrument gain 1200 for the expression of mTq2 (FIG. 15(B)) and GPR68 (FIG. 15(C)) from the X-2 (turquoise), X-3 (red), XI-2 (purple), or XII-5 (green) landing pad. Error bars represent the SEM of four biological replicates.

FIGS. 16(A)-(D) illustrate applications of the four-padded GPCR reporter strains. FIG. 16(A) shows Gα coupling as a function of GPR68 copy number in the 10 four-padded GPCR reporter strains. Data are reported as RFU at an OD₆₀₀ₘₙ of 1.0 and instrument gain 1200 for strains expressing one (X-2), two (X-2, XII-5), three (X-2, XII-5, X-3), and four (X-2, XII-5, X-3, XI-2) copies of GPR68. Error bars represent the SEM of four biological replicates. FIG. 16(B) shows Gα coupling as a function of GPR68 copy number in each of the 10 four-padded GPCR reporter strains shown in FIG. 16(A). FIG. 16(C) is a schematic representation using two of the four landing pads for the autocrine activation of the somatostatin receptor SSTR5 (installed in the X-2 pad) with its genetically-encoded peptide agonist SRIF-14 (installed in XII-5 pad). FIG. 16(D) shows autocrine activation of SSTR5 using the panel of 10 GPCR reporter strains illustrated in FIG. 16(C). Data are reported as ARFU (i.e. RFU of each SSTR5/SRIF-14 autocrine strain subtracted from its counterpart untreated SSTR5-only strain) corresponding to an OD₆₀₀ₘₙ of 1.0 and instrument gain 1200. Error bars represent SEM of four biological replicates.

FIGS. 17(A)-(C) illustrate installation and testing of genome-integrated landing pads in the BY4741 strain. FIG. 17(A) shows the four genome-integrated landing pads of the enhanced BY4741 strain. FIG. 17(B) shows validation of the functionality of each landing pad in the four-padded BY4741 strain using mTq2. Data are reported as RFU at an OD₆₀₀ₘₙ of 1.0 and instrument gain 900. The negative control, NC, corresponds to the empty four-padded BY4741 strain. FIG. 17(C) shows validation of each landing pad using confocal microscopy to quantify the fluorescence of mTq2, mRuby3, pHluorin, and mNeonGreen expressed from the X-2, X-3, XI-2, and XII-5 pads, respectively.

FIGS. 18(A)-(C) show autocrine activation of SSTR5 using the panel of 10 GPCR reporter strains. Data are reported as ΔRFU (i.e. RFU of each SSTR5/SRIF-14 autocrine strain subtracted from its counterpart untreated SSTR5-only strain) corresponding to an OD₆₀₀ₘₙ of 1.0 and instrument gain 1200. Error bars represent SEM of four biological replicates.

FIGS. 19(A)-19(B) show that mTq2 was used to confirm the functionality of each pad in the GPCR reporter strains. In all cases, mTq2 fluorescence was brighter in the BY4741 background than the GPCR-Gα_{I} reporter strain background.

### DETAILED DESCRIPTION

The disclosure relates to a plurality of cells, compositions and methods for identifying modulators of a target domain. The cells, compositions and methods comprise a (i) one or more of a target domain gene that specifically binds to a binding partner (ii) one or more of an intracellular chimeric G-protein alpha subunit comprising an endogenous G-protein alpha subunit with a humanized C-terminus; and (iii) one or more of an inducible reporter, wherein the expression of the reporter is dependent on the activation of the target domain encoded by the target domain gene, and wherein the target domain gene comprises a barcode.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

Without limiting the disclosure, a number of embodiments of the disclosure are described herein for purpose of illustration.

In particular embodiments, provided herein are a plurality of cells, wherein each cell comprises (i) one or more of a target domain gene that specifically binds to a binding partner (ii) one or more of an intracellular chimeric G-protein alpha subunit comprising an endogenous G-protein alpha subunit with a humanized C-terminus; and (iii) one or more of an inducible reporter, wherein the expression of the reporter is dependent on the activation of the target domain encoded by target domain gene, and wherein the target domain gene comprises a barcode.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof.

The plurality of cells may comprise cells, each of which contains only one target domain comprising a barcode that can be used to identify the target domain and an inducible reporter that is activated in the same cell. In particular embodiments, the plurality of cells is ns a “mixture” or “multiplex mixture” of many different GPCR-containing cells against a particular ligand. The population of cells may comprise at least or at most 5, 10, 20, 30, 40, 50, 60, 70, 80, 90, 100, 150, 200, 250, 300, 350, 400, 450, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4500, 5000, 6000, 7000, 8000, 9000, 10000.

“Binding partner” refers to an ion, ligand, small molecule, metabolite, aptamer, peptide, or protein that activates the target domain gene.

The target domain gene can encode a membrane channel, a symporter transporter, an antiporter transporter, an ATPase, an enzyme or a receptor. In particular embodiments, the receptor is a G-protein coupled receptor (GPCR).

The term “G-Coupled Protein Receptor” or “GCPR” refers to any member of the large family of transmembrane receptors that typically function to bind molecules outside the cell and activate inside signal transduction pathways, ultimately inducing one or more cellular responses. G protein-coupled receptors are found only in eukaryotes, including yeast and animals.

Binding and activation of a GPCR typically involves signal transduction pathways including the cAMP signal pathway and the phosphatidylinositol signal pathway. When a ligand binds to the GPCR it causes a conformational change in the GPCR, which allows it to act as a guanine nucleotide exchange factor (GEF). The GPCR can then activate an associated G-protein by exchanging its bound GDP for a GTP. The G-protein's a subunit, together with the bound GTP, can then dissociate from the β and γ subunits to further affect intracellular signaling proteins or target functional proteins directly depending on the a subunit type (Gαs, Gαi/o, Gαq/11, Gα12/13).

All GPCRs share a common structure and mechanism of signal transduction. Generally, GPCRs can be grouped into 6 classes based on sequence homology and functional similarity: Class A (or 1) (Rhodopsin-like), Class B (or 2) (Secretin receptor family), Class C (or 3) (Metabotropic glutamate/pheromone), Class D (or 4) (Fungal mating pheromone receptors), Class E (or 5) (Cyclic AMP receptors), Class F (or 6) (Frizzled/Smoothened). The human genome alone encodes thousands of G protein-coupled receptors, many of which are involved in detection of endogenous ligands (e.g., hormones, growth factors, etc.).

In some embodiments, the GPCR is localized to the cell membrane. In some embodiments, the GPCR is localized intracellularly. In some embodiments, the cell lacks an endogenous gene that encodes for a GPCR that is at least 80% identical to the target domain GPCR gene. In some embodiments, the GPCR gene is integrated into the cell's genome. In particular embodiments, the GPCR gene is integrated from a safe harbor locus located in chromosome X, known as X-2. In some embodiments, the inducible reporter is integrated into the cell's genome.

In particular embodiments, the target domain gene comprises a “barcode” or a unique sequence. The barcode is used to uniquely identify or distinguish the target domain. The barcode may be of any suitable length for unambiguously identifying the target domain gene. The length of the barcode sequence is not critical, and may be of any length sufficient to distinguish the barcode sequence from other barcode sequences. In particular embodiments, the target domain gene is heterologous to the yeast system and represents a unique DNA sequence that can be identified by quantitative polymerase chain reaction, NanoString, sequencing, and similar methods.

In some embodiments, the reporter is induced by signal transduction upon activation of the GPCR. In some embodiments, the reporter comprises one or more of a cAMP response element (CRE), a nuclear factor of activated T-cells response element (NFAT-RE), serum response element (SRE), and serum response factor response element (SRF-RE). In some embodiments, the reporter is a transcriptional reporter such as mTurquoise2 (mTq2). In particular embodiments, the mTq2 reporter replaces the pheromone-responsive gene FIG1 open reading frame in the cell.

In some embodiments, the cells are yeast cells Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

In particular embodiments, the cells have disruption of the pheromone pathway such as deletion of FAR1 and SST2. In particular embodiments, the factor arrest protein (FAR1) is deleted to prevent cell-cycle arrest upon pathway activation. In some embodiments, the GTPase-activating protein (SST2) is deleted to sensitize the pheromone pathway by prolonging Gα activation. In some embodiments, the endogenous yeast GPCR STE2 gene is deleted.

In particular embodiments, each cell has an endogenous G-protein alpha subunit with a humanized C-terminus. In particular embodiments, the last five yeast residues of the yeast Gα subunit, Gpa1, is replaced by the last five residues of a human Gα subunit. In some embodiments, activation of the Gα chimera triggers a MAP kinase signaling cascade that drives the expression of the transcriptional reporter, mTurquoise2 (mTq2).

In particular embodiments, each cell has two target domain genes that specifically binds to a binding partner, two intracellular chimeric G-protein alpha subunits comprising an endogenous G-protein alpha subunit with a humanized C-terminus, and two inducible reporters. In particular embodiments, each cell has three target domain genes that specifically binds to a binding partner, three intracellular chimeric G-protein alpha subunits comprising an endogenous G-protein alpha subunit with a humanized C-terminus, and three inducible reporters. In particular embodiments, each cell has four target domain genes that specifically binds to a binding partner, four intracellular chimeric G-protein alpha subunits comprising an endogenous G-protein alpha subunit with a humanized C-terminus, and four inducible reporters.

In some embodiments, provided herein is a method for identifying a compound capable of modulating the activity of a target domain, comprising: (a) contacting the plurality of cells disclosed herein with a compound; (b) determining the activity of the target domain by detecting the reporter; wherein detection of the reporter in the cell indicates that the compound interacts with the target domain.

In one embodiment, a compound is capable of modulating the activity of a target domains when it is capable of affecting directly or indirectly the activity of the domain.

The methods disclosed herein involve identification of a candidate compound which affects in some way the activity of the target domain. The methods also encompass the ability to screen a library of potential candidate compounds, such that compounds can be utilized in further therapeutic development. Many GPCRs bind ligands at multiple recognition sites. Endogenous ligands that bind to primary binding sites are referred to as orthosteric ligands, while those that bind to secondary sites are allosteric modulators. In particular embodiments, the methods allow identification of metabolites that serve as these types of regulators for a variety of GPCRs. In particular embodiments, the methods allow identification of activating ligands, agonists, antagonists, lead compounds, drugs, or portions thereof.

In particular embodiments, determining the activity of the target domain by detecting the reporter is performed using fluorescence activated cell sorting. In particular embodiments, a tracer strain is included in the methods to enable the comparison of different runs and to empirically determine the optimal duration of the sorting procedure. In particular embodiments, FACS gates are used to discern tracer, active, and inactive cell pools.

A safe harbor loci refers to a loci of the host cell located in non-coding regions and possess high gene expression. In some embodiments provided herein is a yeast cell comprising a plurality of exogenous landing pads integrated in the yeast cell's genome, wherein each exogenous landing pad is integrated at a safe harbor genome loci in the yeast cell's genome.

In some embodiments, the host cell comprises between 1 to 4 exogenous landing pads. In some embodiments, the host cell comprises 1, 2 or 3 exogenous landing pads. In some embodiments, the host cell comprises 4 exogenous landing pads.

In some embodiments, the host cell is Saccharomyces cervisiae. In some embodiments, the plurality of exogenous landing pads are integrated at loci X-2, X-3, XI-2, and/or XII-5 of the host cell's genome. In some embodiments, the plurality of exogenous landing pads are integrated sequentially. In some embodiments, the plurality of exogenous landing pads are integrated sequentially in the following order: X-2 , XII-5, X-3, and XI-2.

In some embodiments, the plurality of exogenous landing pads comprise a unique targeting sequence. In some embodiments, the plurality of exogenous landing pads comprise a unique target sequence, a PAM site, buffer DNA.

### EXAMPLES

The Examples that follow are illustrative of specific embodiments of the disclosure, and various uses thereof. They are set forth for explanatory purposes only and should not be construed as limiting the scope of the disclosure in any way.

### Example 1: High-Throughput CRISPR Genome Editing Pipeline

### i. Materials and Methods

Media. The different media types used in the examples are detailed in Table 1.

TABLE 1

| REAGENT or RESOURCE | SOURCE | | IDENTIFIER |
|---|---|---|---|
| Media Formulations | Concentration | | |
| Medium | | | |
| Synthetic Complete Dextrose/Synthetic Complete | SCD/SCD -U | | |
| Dextrose - Uracil Medium | | | |
| Ammonium sulfate | 5.00 | g/L | |
| CSM/CSM-URA supplement mixture | 0.79/0.77 | g/L | |
| Glucose | 20.00 | g/L | |
| Sodium hydroxide | 0.1 | g/L | |
| Yeast nitrogen base without amino acids & | 1.70 | g/L | |
| ammonium sulfate | | | |
| Synthetic Complete Dextrose Low Fluorescence | | | SCD LoFo pH 7.0 |
| Screening Medium | | | |
| Ammonium sulfate | 5.00 | g/L | |
| CSM supplement mixture | 0.79 | g/L | |
| Dextrose | 20.00 | g/L | |
| MES monohydrate | 9.76 | g/L | |
| Potassium phosphate dibasic | 8.72 | g/L | |
| Yeast nitrogen base- Low fluorescence without | 1.70 | g/L | |
| amino acids, folic acid, and riboflavin | | | |
| Potassium hydroxide solution | pH adjustment to 7.0 | | |
| Hydrochloric acid solution | pH adjustment to 7.0 | | |
| Yeast Peptone Dextrose Medium | | | YPD |
| Yeast extract | 10.00 | g/L | |
| Peptone | 20.00 | g/L | |
| D-(+)-Glucose | 10.00 | g/L | |
| 5FOA Counterselection Medium | | | 5FOA |
| Yeast nitrogen base | 6.7 | g/L | |
| CSM supplement mixture | 0.79 | g/L (has 20 mg uracil) | |
| Uracil | 30 | mg/L (50 mg total) | |
| 5FOA | 1 | g/L | |
| Dextrose | 20 | g/L | |
| Bacto Agar | 15 | g/L | |

| | | |
|---|---|---|
| Chemicals, Peptides, and Recombinant Proteins | | |
| (+)-Sodium L-ascorbate | Research Products | S42165 |
| | International | |
| (±)-Epinephrine hydrochloride | Sigma-Aldrich | E4642 |
| (Arg⁸)-vasopressin acetate | Sigma-Aldrich | V9879 |
| 18:1 Lyso PA (LPA) | Avanti Polar Lipids | 857130 |
| 2-amino-1-phenylethanol (PEOA) | Sigma-Aldrich | A72405 |
| 2-arachidonoylglycerol (2-AG) | Sigma-Aldrich | A8973 |
| 3-hydroxyoctanoic acid | Sigma-Aldrich | H3898 |
| Acetylcholine chloride | Sigma-Aldrich | A6625 |
| Adenosine | Sigma-Aldrich | A9521 |
| Agar bacteriological grade | Research Products | 20030 |
| | International | |
| Ammonium sulfate | Sigma-Aldrich | A4915 |
| Androsterone | Sigma-Aldrich | E3375 |
| CSM supplement mixture | MP Biomedicals | SKU 114500012 |
| CSM-URA supplement mixture | MP Biomedicals | SKU 114511212 |
| D-(+)-Glucose | Research Products | G32045 |
| | International | |
| Dehydroepiandrosterone (DHEA) | EMD Millipore | 252805 |
| Dopamine hydrochloride | Sigma-Aldrich | H8502 |
| Ethylenediaminetetraacetate disodium salt | Sigma-Aldrich | E5134 |
| dihydrate (EDTA) | | |
| HU-210 | Supelco | S-024 |
| Human Endogenous Metabolite Compound Library | MedChemExpress | HY-L030 |
| Hydrochloric acid 5N | J T Baker | 5618-02 |
| Isoprenaline hydrochloride | Sigma-Aldrich | I5627 |
| Kynurenic acid | Sigma-Aldrich | K3375 |
| L-kynurenine | Sigma-Aldrich | K8625 |
| Lithium acetate | Sigma-Aldrich | 517992 |
| Melatonin | Sigma-Aldrich | M5250 |
| MES monohydrate | Research Products | M22040 |
| | International | |
| Nicotinic acid (niacin) | Sigma-Aldrich | N4126 |
| Peptone | Research Products | P20240 |
| | International | |
| Petroselinic acid | Sigma-Aldrich | P8750 |
| Platelet-activating factor (PAF) | Sigma-Aldrich | P7568 |
| Polyethylene glycol 3350 (PEG₃₃₅₀) | Sigma-Aldrich | 88276 |
| Potassium phosphate dibasic | Sigma-Aldrich | P3786 |
| Prostaglandin E2 | Sigma-Aldrich | P5172 |
| Salmon sperm DNA | Invitrogen | 15632-011 |
| Serotonin hydrochloride | Sigma-Aldrich | H9523 |
| Sodium acetate | Sigma-Aldrich | S2889 |
| Sodium ascorbate | Sigma-Aldrich | A7631 |
| Sodium butyrate | Sigma-Aldrich | B5887 |
| Sodium hydroxide | BDH-VWR | BDH9292 |
| Sodium propionate | Sigma-Aldrich | P1880 |
| Sodium succinate | Sigma-Aldrich | 14160 |
| Somatostatin-14 (SRIF-14) | Cayman Chemical | 20809 |
| Sphingosine-1-phosphate (S1P) | Avanti Polar Lipids | LM2144 |
| Trizma base | Sigma-Aldrich | 93352 |
| Tryptamine hydrochloride | Sigma-Aldrich | 246557 |
| Yeast extract | Research Products | Y20020 |
| | International | |
| Yeast nitrogen base - Low fluorescence without | Formedium | CYN6505 |
| amino acids, folic acid and riboflavin | | |
| Yeast nitrogen base without amino acids & | Research Products | Y20060 |
| ammonium sulfate | International | |
| β-alanine | Sigma-Aldrich | 146064 |
| Experimental Models: Organisms/Strains | | |
| BY4741 | Gift from Dohlman lab | BY4741 |
| BY4741 far1Δ sst2Δ ste2Δ fig1Δ::mTq2 X- | This paper | DI dCyFIR P1 I |
| 2: P_{TEF1a}-UnTS-T_{CYC1b} gpal(468- | | |
| 472)(KIGII > ECGLY) | | |
| BY4741 far1Δ sst2Δ ste2Δ fig1Δ::mTq2 X- | This paper | DI dCyFIR P1 O |
| 2: P_{TEF1a}-UnTS-T_{CYC1b} gpal(468- | | |
| 472)(KIGII > GCGLY) | | |
| BY4741 far1Δ sst2Δ ste2Δ fig1Δ::mTq2 X- | This paper | DI dCyFIR P1 T |
| 2: P_{TEF1a}-UnTS-T_{CYC1b} gpal(468- | | |
| 472)(KIGII > DCGLF) | | |
| BY4741 far1Δ sst2Δ ste2Δ fig1Δ::mTq2 X- | This paper | DI dCyFIR P1 Z |
| 2: P_{TEF1a}-UnTS-T_{CYC1b} gpal(468- | | |
| 472)(KIGII > YIGLC) | | |
| BY4741 far1Δ sst2Δ ste2Δ fig1Δ::mTq2 X- | This paper | DI dCyFIR P1 Q |
| 2: P_{TEF1a}-UnTS-T_{CYC1b} gpal(468- | | |
| 472)(KIGII > EYNLV) | | |
| BY4741 far1Δ sst2Δ ste2Δ fig1Δ::mTq2 X- | This paper | DI dCyFIR P1 14 |
| 2: P_{TEF1a}-UnTS-T_{CYC1b} gpal(468- | | |
| 472)(KIGII > EFNLV) | | |
| BY4741 far1Δ sst2Δ ste2Δ fig1Δ::mTq2 X- | This paper | DI dCyFIR P1 15 |
| 2: P_{TEF1a}-UnTS-T_{CYC1b} gpal(468- | | |
| 472)(KIGII > EINLL) | | |
| BY4741 far1Δ sst2Δ ste2Δ fig1Δ::mTq2 X- | This paper | DI dCyFIR P1 12 |
| 2: P_{TEF1a}-UnTS-T_{CYC1b} gpal(468- | | |
| 472)(KIGII > DIMLQ) | | |
| BY4741 far1Δ sst2Δ ste2Δ fig1Δ::mTq2 X- | This paper | DI dCyFIR P1 13 |
| 2: P_{TEF1a}-UnTS-T_{CYC1b} gpal(468- | | |
| 472)(KIGII > QLMLQ) | | |
| BY4741 far1Δ sst2Δ ste2Δ fig1Δ::mTq2 X- | This paper | DI dCyFIR P1 S |
| 2: P_{TEF1a}-UnTS-T_{CYC1b} gpal(468- | | |
| 472)(KIGII > QYELL) | | |
| Software and Algorithms | | |
| GraphPad Prism 7 | GraphPad Software | https://www.graphpad.com |
| BLAST | National Center for | https://blast.ncbi.nlm.nih.gov |
| | Biotechnological | |
| | Information | |
| Python v2.7.16 | Python Software Foundation | https://www.python.org |
| Primer3 | Open Source | https://primer3.org |
| JMP | SAS | https://jmp.com |
| Zen | Zeiss | https://www.zeiss.com |
| biomekController.py | This study | N/A |
| matrixMinusPlus.py | This study | N/A |
| matrixMinusPlusParse.py | This study | N/A |
| libraryScreener.py | This study | N/A |
| hitPickerByZ.py | This study | N/A |
| biomekYeastTrafo.py | This study | N/A |
| qPcrPrimerDesigner.py | This study | N/A |
| biomekQpcrDeconvo.py | This study | N/A |
| qPcrDeconvoParse.py | This study | N/A |

CRISPR transformation reactions. CRISPR edits in yeast were done by co-transforming plasmids containing the CRISPR machinery (Cas9 endonuclease and guide RNA) and DNA payloads containing homology arms typically within 30 bp of the double-stranded DNA break made by Cas9 at the targeted genome locus. The transformations were performed both on an individual basis and in high-throughput format.

Individual CRISPR transformation reactions. Five milliliters of cells were grown to mid-log phase (OD=0.2-1.0) in YPD medium. Cells were harvested by centrifugation and washed with 5 mL TE buffer (10 mM Tris, 1 mM EDTA), then harvested again and washed with 5 mL lithium acetate mix buffer (LiOAc mix; 10 mM Tris, 1 mM EDTA, 100 mM LiOAc). Cells were harvested by centrifugation and resuspended in 200 μL LiOAc mix buffer. CRISPR vector(s) (300 ng) and DNA payload (4-5 μg) were combined with salmon sperm DNA (100 μg) in a mix with 50 μL cells and 350 μL PEG mix (10 mM Tris, 1 mM EDTA, 100 mM LiOAc, 40% PEG₃₃₅₀). This mixture was incubated at room temperature for 30 minutes before addition of 24 μL DMSO and a 15 minute heat shock at 42° C. Following heat shock, cells were harvested by centrifugation at 8000×g for 1 minute, resuspended in 200 μL YPD and spread on selective media plates poured in petri-dishes with a 100 mm diameter. This protocol is sufficient for 4 individual CRISPR transformations and is scalable.

High-throughput CRISPR transformation reactions. Hundreds of new yeast strains were engineered using a miniaturized version of the protocol for “Individual CRISPR transformation reactions”. Both approaches used the same culture and plate media compositions. For the miniaturized protocol, the necessary CRISPR vector(s) (150 ng) and DNA payload (4-5 μg) for many transformation reactions (typically 80-100 at a time) were combined in a mixture with 50 μL cells and 175 μL PEG mix in individual wells of a 96-well plate (CytoOne CC7672-7596). This mixture was incubated for 30 minutes at room temperature before addition of 12 μL DMSO and a 15 minute heat shock at 42° C. Cells were harvested by centrifugation and resuspended in 100 μL YPD. 30 μL transformant resuspension was distributed over miniaturized selective media plates poured in a 22.1 mm 12-well microplates (CytoOne CC7672-7512). Each step was performed on our Biomek NXp liquid-handling robot driven by our custom Python code. Colonies were picked into SCD-U media.

Counterselection to remove CRISPR vectors. An advantage of CRISPR in yeast is the ability to engineer a genome modification without exhausting available auxotrophic markers. In the examples, CRISPR vectors conferring URA selectivity were primarily used, which could be removed via counter-selection on 5FOA plates after the desired genome edit was confirmed. In cases where two CRISPR vectors where used, such as gene deletions, the CRISPR vector conferring URA selectivity was removed as usual by counter-selection on 5FOA and the CRISPR vector conferring LEU selectivity was naturally lost after several generations of outgrowth in non-selective media. As a result, the base BY4741 genotype remained unchanged, despite the many changes introduced into the genomes of the GPCR-Gα reporter strains.

CRISPR guide RNA plasmid design. Previously described methods and base CRISPR vectors pML104 (Addgene #67638), pML107 (Addgene #67639), and pT040 (Addgene #67640) (Laughery, et al., (2015). Yeast 32, 711-720) were used to construct all genomically targeted guide RNA plasmids used in this study. Briefly, pML104 and pML107 plasmids both contain the Cas9 endonuclease ORF, a gRNA scaffold flanked by a SNR52 promoter and SUP4 terminator, and an auxotrophic marker (URA and LEU, respectively). Plasmid pT040 contains the same gRNA scaffold as pML104 and pML107, as well as a URA auxotrophic marker.

CRISPR DNA payload design. In every CRISPR transformation reaction, the necessary CRISPR vector(s) were co-transformed with a DNA sequence that serves to both 1) patch the locus-specific double-stranded DNA break caused by the Cas9 endonuclease and 2) incorporate the desired gene deletion, edit, replacement, or knock-in. This DNA sequence, referred to as a DNA payload, contains both the DNA required for the desired CRISPR change (usually a heterologous gene or gene fragment) and important design features for targeting the intended genome location (homology arms) and preventing continued Cas9 cutting once the genome has been altered (PAM silencing).

Homology arm design. The DNA payloads were flanked by homology arms that typically contained 60 bp of genome sequence upstream and downstream of the targeted genome locus. These homology arms were introduced by PCR amplification (using primers with overhangs containing the necessary homology), or by including the sequence homology directly in the designs of synthetic payloads (e.g. gBlocks and synthetic DNA constructs cloned into storage vectors). Based on empirical observations from thousands of CRISPR edits, homology arms less than 60 bp were not used to prevent diminished CRISPR efficiency.

PAM silencing design. Once a DNA payload has been integrated into the yeast genome, Cas9 endonuclease will continue to create a double-stranded break at the targeted genome locus if the PAM site is not removed by the genome edit. Such constant cutting can reduce CRISPR efficiency due to its effect as a cytotoxic stress and as a mechanism for reversing the desired genome edit. In such cases where PAM silencing was needed, continuous genome cutting was prevented by including a point mutation in the portion of the homology arm that corresponds to the PAM site. This process was called PAM silencing. In cases where PAM silencing occurred within the open reading frame of a protein, an alternative amino acid codon that removes the PAM site was used in place of the original codon in the homology arm.

The collection of PAM-silenced homology arms used to deliver the various CRISPR payloads is listed in Table 2. Deletion, editing, and replacement of all genes was verified by PCR gel electrophoresis using the relevant primers.

TABLE 2

| Construct | | |
|---|---|---|
| name | Type | Sequence (5'-3') |
| FAR1 | gBlock | TGTCTTGAGAGTGTATATTATCTTATCTATTCAAAAAATTTC |
| DP | | TATTTACTTTTATATTTCTTGACCATCCTTTACACAAAGTCT |
| | | ATAGATCCACTGGAAAGCTTCGTGGGCGTAAGAAGGCAAT |
| | | CTATTATAGTTCGGGAATCGAGGCCCGTATTTCGAGGCTTT |
| | | TGCTTTTCCTTTTTTTTTTTTCGTTTCTCCACGTCTATACTAC |
| | | GCAATGACTGAATATATATAATGCGTCGTAAATAGCAGTT |
| | | AATGTATAAATA |
| Ste2 DP | gBlock | CTTCAAAGCAATACGATACCTTTTCTTTTCACCTGCTCTGGC |
| | | TATAATTATAATTGGTTACTTAAAAATGCACCGTTAAGAAC |
| | | CATATCCAAGAATCAAAATCAAAATTTACGGCTTTGAAAA |
| | | AGTAATTTCGTGACCTTCGGTATAAGGTTACTACTAGATTC |
| | | AGGTGCTCATCAGATGCACCACATTCTCTATAAAAAAA |
| GPA1_ | gBlock | TTTTTGAGCTTGAATAAGACAAACAAACCAATCTACGTGA |
| chimera_ | | AACGAACCTGCGCTACCGATACCCAAACTATGAAGTTCGT |
| GNAI3 | | ATTGAGTGCAGTCACTGATCTAATCATCCAGCAAAACCTTA |
| | | AAGAATGTGGTTTGTATTGAAGGAACTGTATAATTAAAGT |
| | | AGTGTTTAGATACGTAAATTCTGTTTCCGAAGATGCAAGAA |
| | | GGAGCAGCAGCACCAGAAAAAATTACTATTTTTCTTCTCCA |
| | | TTAGAGTCTATGATGGAA |
| GPA1_ | gBlock | TTTTTGAGCTTGAATAAGACAAACAAACCAATCTACGTGA |
| chimera_ | | AACGAACCTGCGCTACCGATACCCAAACTATGAAGTTCGT |
| GNAO | | ATTGAGTGCAGTCACTGATCTAATCATCCAGCAAAACCTTA |
| | | AAGGTTGTGGTTTGTATTGAAGGAACTGTATAATTAAAGTA |
| | | GTGTTTAGATACGTAAATTCTGTTTCCGAAGATGCAAGAAG |
| | | GAGCAGCAGCACCAGAAAAAATTACTATTTTTCTTCTCCAT |
| | | TAGAGTCTATGATGGAA |
| GPA1_ | gBlock | TTTTTGAGCTTGAATAAGACAAACAAACCAATCTACGTGA |
| chimera_ | | AACGAACCTGCGCTACCGATACCCAAACTATGAAGTTCGT |
| GNAT12 | | ATTGAGTGCAGTCACTGATCTAATCATCCAGCAAAACCTTA |
| 3_I12 | | AAGATTGCGGTTTGTTTTGAAGGAACTGTATAATTAAAGTA |
| | | GTGTTTAGATACGTAAATTCTGTTTCCGAAGATGCAAGAAG |
| | | GAGCAGCAGCACCAGAAAAAATTACTATTTTTCTTCTCCAT |
| | | TAGAGTCTATGATGGAA |
| GPA1_ | gBlock | TTTTTGAGCTTGAATAAGACAAACAAACCAATCTACGTGA |
| chimera_ | | AACGAACCTGCGCTACCGATACCCAAACTATGAAGTTCGT |
| GNAZ | | ATTGAGTGCAGTCACTGATCTAATCATCCAGCAAAACCTTA |
| | | AATATATCGGTTTGTGCTGAAGGAACTGTATAATTAAAGTA |
| | | GTGTTTAGATACGTAAATTCTGTTTCCGAAGATGCAAGAAG |
| | | GAGCAGCAGCACCAGAAAAAATTACTATTTTTCTTCTCCAT |
| | | TAGAGTCTATGATGGAA |
| GPA1_ | gBlock | TTTTTGAGCTTGAATAAGACAAACAAACCAATCTACGTGA |
| chimera_ | | AACGAACCTGCGCTACCGATACCCAAACTATGAAGTTCGT |
| GNAQ_1 | | ATTGAGTGCAGTCACTGATCTAATCATCCAGCAAAACCTTA |
| 1 | | AAGAATATAATTTGGTTTGAAGGAACTGTATAATTAAAGT |
| | | AGTGTTTAGATACGTAAATTCTGTTTCCGAAGATGCAAGAA |
| | | GGAGCAGCAGCACCAGAAAAAATTACTATTTTTCTTCTCCA |
| | | TTAGAGTCTATGATGGAA |
| GPA1_ | gBlock | TTTTTGAGCTTGAATAAGACAAACAAACCAATCTACGTGA |
| chimera_ | | AACGAACCTGCGCTACCGATACCCAAACTATGAAGTTCGT |
| GNA14 | | ATTGAGTGCAGTCACTGATCTAATCATCCAGCAAAACCTTA |
| | | AAGAATTTAATTTGGTTTGAAGGAACTGTATAATTAAAGTA |
| | | GTGTTTAGATACGTAAATTCTGTTTCCGAAGATGCAAGAAG |
| | | GAGCAGCAGCACCAGAAAAAATTACTATTTTTCTTCTCCAT |
| | | TAGAGTCTATGATGGAA |
| GPA1_ | gBlock | TTTTTGAGCTTGAATAAGACAAACAAACCAATCTACGTGA |
| chimera_ | | AACGAACCTGCGCTACCGATACCCAAACTATGAAGTTCGT |
| GNA15 | | ATTGAGTGCAGTCACTGATCTAATCATCCAGCAAAACCTTA |
| | | AAGAAATTAATTTGTTGTGAAGGAACTGTATAATTAAAGT |
| | | AGTGTTTAGATACGTAAATTCTGTTTCCGAAGATGCAAGAA |
| | | GGAGCAGCAGCACCAGAAAAAATTACTATTTTTCTTCTCCA |
| | | TTAGAGTCTATGATGGAA |
| GPA1_ | gBlock | TTTTTGAGCTTGAATAAGACAAACAAACCAATCTACGTGA |
| chimera_ | | AACGAACCTGCGCTACCGATACCCAAACTATGAAGTTCGT |
| GNA12 | | ATTGAGTGCAGTCACTGATCTAATCATCCAGCAAAACCTTA |
| | | AAGATATTATGCTACAATGAAGGAACTGTATAATTAAAGT |
| | | AGTGTTTAGATACGTAAATTCTGTTTCCGAAGATGCAAGAA |
| | | GGAGCAGCAGCACCAGAAAAAATTACTATTTTTCTTCTCCA |
| | | TTAGAGTCTATGATGGAA |
| GPA1_ | gBlock | TTTTTGAGCTTGAATAAGACAAACAAACCAATCTACGTGA |
| chimera_ | | AACGAACCTGCGCTACCGATACCCAAACTATGAAGTTCGT |
| GNA13 | | ATTGAGTGCAGTCACTGATCTAATCATCCAGCAAAACCTTA |
| | | AACAATTGATGTTGCAATGAAGGAACTGTATAATTAAAGT |
| | | AGTGTTTAGATACGTAAATTCTGTTTCCGAAGATGCAAGAA |
| | | GGAGCAGCAGCACCAGAAAAAATTACTATTTTTCTTCTCCA |
| | | TTAGAGTCTATGATGGAA |
| GPA1_ | gBlock | TTTTTGAGCTTGAATAAGACAAACAAACCAATCTACGTGA |
| chimera_ | | AACGAACCTGCGCTACCGATACCCAAACTATGAAGTTCGT |
| GNAS_L | | ATTGAGTGCAGTCACTGATCTAATCATCCAGCAAAACCTTA |
| | | AACAATATGAATTGTTGTGAAGGAACTGTATAATTAAAGT |
| | | AGTGTTTAGATACGTAAATTCTGTTTCCGAAGATGCAAGAA |
| | | GGAGCAGCAGCACCAGAAAAAATTACTATTTTTCTTCTCCA |
| | | TTAGAGTCTATGATGGAA |
| CHRM1 | gBlock | ATGAATACAAGCGCTCCTCCAGCGGTTTCCCCAAATATTAC |
| Δil3 | | GGTGCTGGCACCTGGGAAAGGACCATGGCAAGTTGCTTTT |
| | | ATCGGTATCACCACCGGCTTGCTCTCCTTGGCAACAGTCAC |
| | | GGGCAATTTGCTCGTACTGATCTCATTTAAGGTGAACACAG |
| | | AGCTGAAAACAGTGAACAACTACTTCTTGTTGAGTTTGGCC |
| | | TGTGCGGACCTTATTATCGGAACGTTTTCAATGAATCTGTA |
| | | TACAACCTATCTCCTGATGGGCCACTGGGCGCTGGGAACCC |
| | | TTGCTTGCGACCTTTGGTTGGCCCTTGATTACGTGGCGAGC |
| | | AACGCAAGCGTCATGAATCTCCTGTTGATATCATTTGACAG |
| | | GTATTTTTCTGTTACCCGGCCCCTGAGCTACAGAGCTAAAC |
| | | GAACTCCACGACGCGCTGCACTGATGATTGGTCTGGCCTGG |
| | | CTGGTCAGTTTTGTCCTGTGGGCCCCTGCCATACTGTTTTGG |
| | | CAGTACCTGGTGGGAGAGCGCACCGTGCTTGCAGGTCAGT |
| | | GCTATATACAGTTTCTGTCTCAGCCCATAATCACATTCGGC |
| | | ACCGCGATGGCGGCATTTTACTTGCCTGTGACTGTTATGTG |
| | | TACTCTGTATTGGCGAATTTACCGCGAAACCGAGAATAGA |
| | | GCCAGGGAACTTGCTGCACTGCAGGGTTCAGAGACAAAGG |
| | | AACAGCTGGCGAAGCGGAAAACCTTCAGCCTCGTGAAGGA |
| | | AAAGAAAGCCGCACGAACCCTGTCCGCTATCTTGCTGGCTT |
| | | TCATACTGACCTGGACGCCTTATAACATTATGGTGCTGGTC |
| | | TCCACCTTTTGTAAAGATTGCGTCCCAGAGACACTTTGGGA |
| | | GCTGGGGTACTGGCTGTGCTATGTGAACTCCACAATTAACC |
| | | CAATGTGTTATGCCTTGTGTAATAAAGCGTTCAGAGATACA |
| | | TTCCGGCTTCTCCTTCTGTGCCGGTGGGATAAGAGGCGCTG |
| | | GCGCAAAATCCCCAAACGGCCCGGGTCAGTTCACAGAACA |
| | | CCTAGCAGACAATGTTAA |
| CHRM3 | gBlock | ATGACGCTGCATAATAATTCAACCACAAGCCCTCTGTTCCC |
| Δil3 | | TAACATCAGTAGTAGCTGGATACACTCTCCTAGCGACGCCG |
| | | GACTTCCTCCCGGAACCGTGACCCACTTTGGGTCATATAAC |
| | | GTCTCTCGGGCCGCAGGCAATTTTTCTTCCCCAGATGGGAC |
| | | AACCGACGATCCGCTCGGCGGCCACACTGTATGGCAGGTG |
| | | GTTTTCATAGCCTTTCTGACCGGCATTCTGGCTCTGGTCAC |
| | | AATCATCGGCAATATTCTGGTAATTGTGTCCTTCAAGGTAA |
| | | ACAAACAACTGAAGACTGTCAATAACTACTTTCTGCTTAGT |
| | | TTGGCATGCGCCGATCTGATTATCGGAGTGATAAGTATGAA |
| | | TCTCTTCACTACTTACATCATTATGAACAGATGGGCTCTGG |
| | | GTAACTTGGCTTGCGACCTGTGGCTCGCAATTGACTATGTC |
| | | GCAAGCAACGCCAGTGTGATGAACCTTCTGGTTATTAGCTT |
| | | CGACCGCTATTTTAGCATTACACGCCCTCTGACCTACAGAG |
| | | CGAAACGCACTACAAAGCGGGCAGGAGTTATGATCGGCCT |
| | | GGCCTGGGTCATTTCCTTCGTGCTCTGGGCACCAGCCATCT |
| | | TGTTCTGGCAGTATTTCGTCGGTAAGAGGACAGTCCCACCA |
| | | GGCGAGTGCTTCATCCAATTTTTGTCCGAGCCAACCATAAC |
| | | ATTCGGCACAGCCATCGCGGCATTTTACATGCCAGTGACTA |
| | | TCATGACCATTCTGTACTGGAGAATCTACAAAGAAACCGA |
| | | AAAGCGGACAAAGGAGTTGGCAGGGCTTCAGGCATCAGGC |
| | | ACAGAGACCCGGTCACAGATAACCAAGCGCAAGCGAATGT |
| | | CACTCGTGAAAGAAAAAAAAGCAGCACAGACATTGAGCGC |
| | | CATCTTGCTCGCTTTTATCATAACATGGACACCCTACAATA |
| | | TCATGGTCCTCGTGAACACTTTCTGCGACTCCTGTATTCCTA |
| | | AGACTTTCTGGAACCTCGGCTACTGGCTGTGCTATATCAAC |
| | | AGTACAGTTAACCCTGTGTGCTACGCTCTCTGTAACAAGAC |
| | | CTTTAGGACCACGTTCAAGATGCTCCTCCTGTGCCAGTGTG |
| | | ACAAAAAGAAACGACGAAAGCAGCAATATCAGCAGCGAC |
| | | AGAGTGTGATCTTTCATAAACGGGCGCCAGAACAAGCGCT |
| | | CTAA |
| CHRM5 | gBlock | ATGGAGGGCGACTCATATCACAATGCCACGACTGTGAACG |
| Δil3 | | GCACACCCGTCAACCACCAGCCACTTGAAAGGCACAGGCT |
| | | TTGGGAGGTAATAACCATAGCCGCAGTGACTGCAGTAGTT |
| | | AGTCTGATTACCATTGTTGGCAATGTATTGGTCATGATTTC |
| | | TTTTAAGGTTAACAGCCAGCTGAAGACCGTGAATAATTATT |
| | | ATCTCCTTTCCCTGGCATGCGCTGACTTGATCATCGGGATC |
| | | TTTAGCATGAACCTGTATACTACGTACATCCTGATGGGGCG |
| | | CTGGGCACTGGGTTCATTGGCATGTGACTTGTGGCTTGCGC |
| | | TGGATTATGTTGCATCTAACGCCAGCGTAATGAATCTTCTC |
| | | GTGATATCCTTTGACAGATACTTCTCTATTACACGGCCCCT |
| | | GACGTACAGAGCCAAAAGGACCCCCAAACGAGCAGGCATC |
| | | ATGATTGGACTGGCTTGGCTCATATCCTTCATACTCTGGGC |
| | | CCCTGCGATCCTCTGTTGGCAGTATCTCGTGGGGAAAAGGA |
| | | CTGTGCCCCTGGACGAATGCCAGATCCAGTTCCTGAGTGAG |
| | | CCCACAATAACTTTTGGGACCGCCATAGCTGCTTTCTATAT |
| | | TCCGGTATCCGTTATGACCATCCTTTACTGCAGGATCTACA |
| | | GAGAGACAGAGAAAAGGACCAAAGATTTGGCTGATCTTCA |
| | | GGGGTCTGATTCTGTGTCTACTAAAGGACTCAACCCGAATC |
| | | CCAGCCATCAGATGACCAAACGCAAGCGGGTAGTGCTGGT |
| | | TAAAGAAAGGAAAGCTGCACAGACTCTGAGCGCGATTCTG |
| | | CTGGCCTTTATCATTACTTGGACCCCCTATAACATCATGGT |
| | | GCTCGTATCTACTTTCTGTGACAAGTGCGTCCCCGTGACCC |
| | | TGTGGCACCTGGGCTACTGGCTGTGCTACGTTAATTCAACA |
| | | GTGAATCCCATCTGTTATGCCCTTTGTAACCGGACCTTTCG |
| | | CAAAACCTTTAAGATGCTGCTTTTGTGTAGGTGGAAGAAG |
| | | AAAAAAGTCGAAGAAAAGCTTTACTGGCAGGGCAATAGCA |
| | | AATTGCCATAA |
| X2-LP- | PCR | TCCATTTCTTTTTCCTCGGGCAGAGAAACTCGCAGGCAACT |
| CYC 1b | product | TGCTCTCGAAGTGGTCACGCACACACCATAGCTTCAAAATG |
| | | TTTCTACTCCTTTTTTACTCTTCCAGATTTTCTCGGACTCCG |
| | | CGCATCGCCGTACCACTTCAAAACACCCAAGCACAGCATA |
| | | CTAAATTTCCCCTCTTTCTTCCTCTAGGGTGTCGTTAATTAC |
| | | CCGTACTAAAGGTTTGGAAAAGAAAAAAGAGACCGCCTCG |
| | | TTTCTTTTTCTTCGTCGAAAAAGGCAATAAAAATTTTTATC |
| | | ACGTTTCTTTTTCTTGAAAATTTTTTTTTTTGATTTTTTTCTC |
| | | TTTCGATGACCTCCCATTGATATTTAAGTTAATAAACGGTC |
| | | TTCAATTTCTCAAGTTTCAGTTTCATTTTTCTTGTTCTATTAC |
| | | AACTTTTTTTACTTCTTGCTCATTAGAAAGAAAGCATAGCA |
| | | ATCTAATCTAAGTTTTAATTACAATTGCGTAAGTGGCCCCT |
| | | AGCGGGGCAGTGGTAATTAGTTATGTCACGCTTACATTCAC |
| | | GCCCTCCTCCCACATCCGCTCTAACCGAAAAGGAAGGAGT |
| | | TAGACAACCTGAAGTCTAGGTCCCTATTTATTTTTTTTAATA |
| | | GTTATGTTAGTATTAAGAACGTTATTTATATTTCAAATTTTT |
| | | CTTTTTTTTCTGTACAAACGCGTGTACGCATGTAACATTAT |
| | | ACTGAAAACCTTGCTTGAGAAGGTTTTGGGACGCTCGAAG |
| | | GCTTTAATTTGGCATAATCGGCCCTCACAGAGGGATCCCGT |
| | | TACCCATCTATGCTGAAGATTTATCATACT |
| fig1- | PCR | GCTTGTCTTTGGTAGAAGAAATTATAGTAAACAAACAAAC |
| mTq2 | product | AAACAAACAAAAAAAAAAAAAAAAAAatgtctaaaggtgaagaattgtt |
| | | tactggtgttgttccaattttggttgaattggatggtgatgttaatggtcataaattttctgtttctggtga |
| | | aggtgaaggtgatgctacttatggtaaattgactttgaaatttatttgtactactggtaaattgccagttc |
| | | catggccaactttggttactactttgtcttggggtgttcaatgttttgctagatatccagatcatatgaaa |
| | | caacatgatttttttaaatctgctatgccagaaggttatgttcaagaaagaactattttttttaaagatgat |
| | | ggtaattataaaactagagctgaagttaaatttgaaggtgatactttggttaatagaattgaattgaaa |
| | | ggtattgattttaaagaagatggtaatattttgggtcataaattggaatataattatttttctgataatgttt |
| | | atattactgctgataaacaaaaaaatggtattaaagctaattttaaaattagacataatattgaagatg |
| | | gtggtgttcaattggctgatcattatcaacaaaatactccaattggtgatggtccagttttgttgccag |
| | | ataatcattatttgtctactcaatctgctttgtctaaagatccaaatgaaaaaagagatcatatggttttg |
| | | ttggaatttgttactgctgctggtattactttgggtatggatgaattgtataaataaTTTTATCCT |
| | | CAAATAAACATATAAGTTTTGAGCGGATATTTCAGAATGTC |
| | | AATTTTTAAAAGTAA |
| fus1- | PCR | TACGACATCCTTTATCTTTTTTCCTTTAAGAGCAGGATATA |
| mTq2 | product | AGCCATCAAGTTTCTGAAAATCAAAatgtctaaaggtgaagaattgtttact |
| | | ggtgttgttccaattttggttgaattggatggtgatgttaatggtcataaattttctgtttctggtgaaggt |
| | | gaaggtgatgctacttatggtaaattgactttgaaatttatttgtactactggtaaattgccagttccatg |
| | | gccaactttggttactactttgtcttggggtgttcaatgttttgctagatatccagatcatatgaaacaa |
| | | catgatttttttaaatctgctatgccagaaggttatgttcaagaaagaactattttttttaaagatgatggt |
| | | aattataaaactagagctgaagttaaatttgaaggtgatactttggttaatagaattgaattgaaaggt |
| | | attgattttaaagaagatggtaatattttgggtcataaattggaatataattatttttctgataatgtttatat |
| | | tactgctgataaacaaaaaaatggtattaaagctaattttaaaattagacataatattgaagatggtgg |
| | | tgttcaattggctgatcattatcaacaaaatactccaattggtgatggtccagttttgttgccagataat |
| | | cattatttgtctactcaatctgctttgtctaaagatccaaatgaaaaaagagatcatatggttttgttgga |
| | | atttgttactgctgctggtattactttgggtatggatgaattgtataaataaTGAAAATAATA |
| | | TTGACGTTCGCATTTAATCTATACCTATAATTCTGTACTTAT |
| | | ATACTGTTCCTT |

Design of the X-2 CRISPR-addressable expression cassette. Random 20 base pair (bp) guide sequences were generated using a custom Python program. Using the BLAST algorithm (Altschul, et al., (1990). J Mol Bio! 215, 403-410), the uniqueness of each guide sequence was tested against a locally built BLAST database for the updated release of the S. cerevisiae BY4741 genome (BY4741_Toronto_2012) available via www.yeastgenome.org (Cherry et al., (2012). Nucleic Acids Res 40, D700-705). Using the established threshold for avoiding off-target Cas9 cutting (DiCarlo, J. E., et al., (2013). Nucleic Acids Res 41, 4336-4343), guide sequences with more than three mismatched bases were identified and used as synthetic unique targeting sites (UnTS). The CRISPR-addressable expression cassette, referred to as a landing pad, was designed to include one of these UnTS sequences (5′-TTGCGTAAGTGGCCCCTAGC-3′) preceding a protospacer adjacent motif (PAM) site (5′-GGG-3′) flanked upstream by a constitutive TEF1 promoter (Partow, S., et al., (2010). Yeast 27, 955-964) and downstream by a CYC1 terminator variant, CYC1b, a corrected version that leads to higher expression output than other CYC1 terminator variants (Curran,.et al., (2013). Metab Eng 19, 88-97). Lastly, the landing pad was extended to include 500 bp homology arms to the known yeast X-2 safe harbor locus on chromosome X (Mikkelsen, et al., (2012). Metab Eng 14, 104-111; Ronda,et al., (2015). Microb Cell Fact 14, 97). The X-2 landing pad sequence synthetically constructed was ordered and cloned into the pMARQ vector by ThermoFisher. The sequence of the X-2 landing pad with homology arms is available in Table 2.

Engineering CRISPR-optimized yeast strains for human GPCR studies. The major steps used to build the 10 base GPCR-Gα reporter strains are summarized below. CRISPR was used to make every gene deletion, replacement, edit, and knock-in in this study (see “CRISPR transformation reaction” method for further detail).

Deletion of signaling components to sensitize the pheromone pathway. In the first steps of strain engineering efforts, the DI2Δ strain was created by sequentially deleting the pheromone pathway components FAR1 and SST2. The factor arrest protein (FAR1) was deleted to prevent cell-cycle arrest upon pathway activation and the GTPase-activating protein (SST2) was deleted to sensitize the pheromone pathway by prolonging Gα activation. The CRISPR gene deletion procedure employed two CRISPR vectors, pML107 and pT040, each having their own selectable markers LEU and URA. Vector pT040 contained a guide RNA sequence that targeted the N-terminal/C-terminal region of the gene to be deleted. These vectors were co-transformed with DNA payload comprising homology arms generally having 60-100 bp of sequence immediately upstream and downstream of the targeted open reading frame.

Installation of the mTq2 transcriptional reporter. Following the creation of the DI2Δ strain, the pheromone-responsive gene FIG1 open reading frame was replaced with the cyan fluorescence protein mTq2. For the CRISPR gene deletion procedure, the FIG1 open reading frame was replaced with the mTq2 gene using two CRISPR vectors pML107 and pT040, each having their own selectable markers LEU and URA. Vectors pML107 and pT040 contained a guide RNA sequence that targeted the N-terminal/C-terminal region of the FIG1 gene. These vectors were co-transformed with DNA payload comprising homology arms having 60 bp of sequence immediately upstream and downstream of FIG1 open reading frame. The resultant genotype of this strain, referred to as DI2Δ fig1Δ::mTq2, was BY4741 far1Δ sst2Δ fig1Δ::mTq2.

Deleting the endogenous yeast GPCR STE2 gene. Following the creation of the DI2Δ fig1Δ::mTq2 strain the native yeast GPCR gene (STE2) was deleted using the same plasmids and procedure described in the section Deletion of signaling components to sensitize the pheromone pathway. This new strain, DI3Δ fig1Δ::mTq2, had the genotype BY4741 far1Δ sst2Δste2Δ fig1Δ::mTq2.

Installation of the X-2 landing pad. Following the creation of the DI3Δ fig1Δ::mTq2 strain, the CRISPR-addressable expression cassette was installed (see “Creating the CRISPR-addressable expression cassette into the X-2 locus” for details) into the X-2 locus of chromosome X. To install the X-2 landing pad into the X-2 safe harbor locus, the landing pad sequence from the pMARQ vector was PCR amplified and co-transformed the resultant DNA payload with the CRISPR vector pML104 X2. The resultant genotype of this strain, referred to as DI3Δ fig1Δ::mTq2 P1, was BY4741 far1Δ sst2Δ ste2Δ fig1Δ::mTq2 X-2:P_{TEF1a}-UnTS-T_{CYC1b}b.

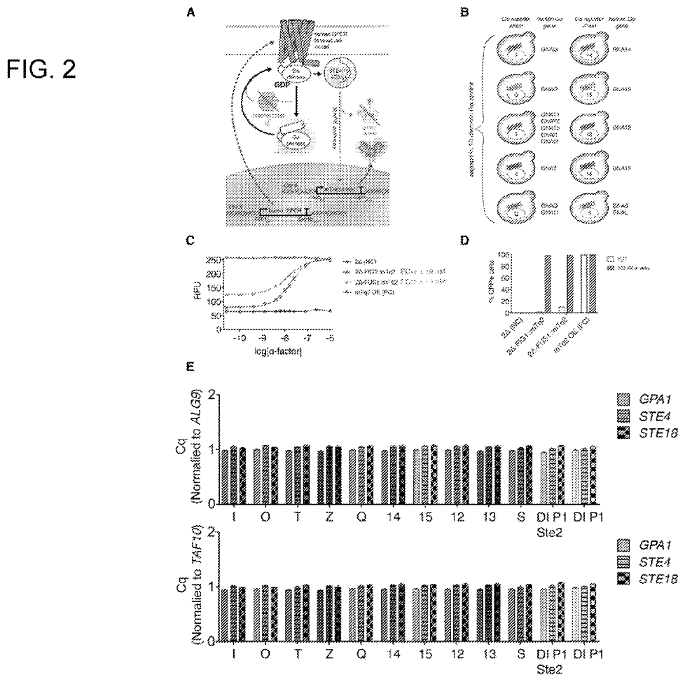

Genome-editing to create humanized yeast C-terminal Gα chimeras. To build the panel of 10 GPCR-Gα base reporter strains, 10 different versions of our DI3Δ fig1Δ::mTq2 P1 strain were created, each having its own unique Gα C-terminal yeast/human chimera. In each Gα chimera, the last five yeast residues of the yeast Gα subunit, Gpa1, were replaced by the last five residues of a human Gα subunit (see FIG. 2 and Table 1 for sequence details). Due to C-terminal degeneracies, all 16 human Gα genes could be represented by 10 Gα C-terminal chimeras. codon-optimized DNA payload for each Gα chimeric sequence was designed as a gBlock gene fragment (Integrated DNA Technologies) comprising the 15 bp sequence of a human Gα C-termini flanked by 123 bp homology arms that targeted the C-terminus of the yeast Gα subunit sequence. These synthetic DNA payloads were co-transformed with the CRISPR vectors pML107 and pT040 GPA1:1373, each having their own selectable markers LEU and URA. The resultant genotypes of these strains, which referred to as DI dCyFIR P1 I, DI dCyFIR P1 O, DI dCyFIR P1 T, DI dCyFIR P1 Z, DI dCyFIR P1 Q, DI dCyFIR P1 14, DI dCyFIR P1 15, DI dCyFIR P1 12, DI dCyFIR P1 13, and DI dCyFIR P1 S.

Installation of human GPCRs into X-2 CRISPR-addressable expression cassette. All human GPCR DNA sequences were lifted from the Presto-TANGO plasmid library (Kroeze, et al., (2015). Nat Struct Mol Biol 22, 362-369), using primers to PCR amplify only the GPCR open reading frame, avoiding the additional N- and C-terminal DNA sequence elements in the Presto-TANGO plasmid constructs. Using two rounds of PCR amplification, each GPCR sequence was extended with homology arms corresponding to sequences within the TEF promoter and CYC lb terminator of the X-2 landing pad. For the first round of PCR, receptor-specific primers having ˜45 bp homology overhangs were used. In a second round of PCR, universal primers to extend both homology arms to a final length of 60 bp were used. With the exception of the muscarinic receptors CHRM1, CHRM3, and CHRM5, native GPCR sequences were used (i.e. no affinity tags or localization sequences were added). However, residues corresponding to the third intracellular loop (iL3) of CHRM1, CHRM3, and CHRMS were deleted to reproduce iL3 deletion results that were previously published in a similar yeast system (Erlenbach, et al., (2001) J Neurochem 77, 1327-1337). As with these original studies, full-length CHRM1, CHRM3, and CHRM5 did not functionally express in the system. The CHRM1, CHRM3, and CHRMS sequences in Table 1 correspond to the iL3 loop deletion variants. To install human GPCRs into the X-2 landing pad, the amplified GPCR PCR product with 60 bp homology arms was co-transformed with the CRISPR vector pML104 X2 UnTS using the approach described in “CRISPR transformation reactions”. Because each human GPCR was installed into all 10 base GPCR-Gα reporter strains, a library of 300 new GPCR-Gα reporter strains barcoded with a human GPCR were produced.

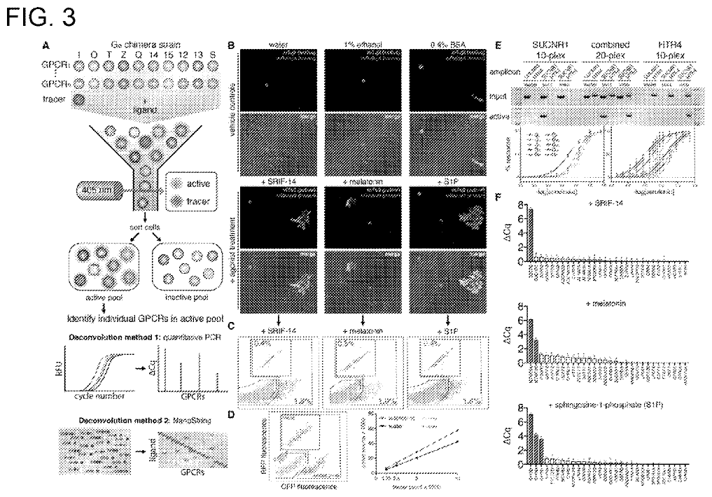

Engineering positive controls strains for dCyFIRplex development. To establish the dCyFIRplex FACS gating procedure a set of 10 base GPCR-Gα reporter strains were used (mixed as a 10-plex) as a negative control (inactive bin in FIG. 3D). To build the positive control strains for cyan (mTq2) and red (mRuby3 tracer) fluorescence, a X-2 landing pad was first installed into the wild-type BY4741 strain using the same procedure described in “Installation of the X-2 landing pad”. Next mTq2 (from a pFA6-link-mTurquoise2-CaURA3MX vector [Addgene #86424]) and mRuby3 (from a pNCS-mRuby3 vector [Addgene #74324]) was PCT amplified using primers containing ˜45 bp homology arms corresponding to sequences within the TEF promoter and CYC lb terminator of the X-2 landing pad. In a second round of PCR, universal primers were used to extend both homology arms to a final length of 60 bp. The PCR-amplified genes were then installed into their respective X-2 landing pads by co-transformation with the CRISPR vector pML104 X2 UnTS using the approach described in “CRISPR transformation reactions”. Integration into the X-2 landing pad was confirmed both by PCR and a marked increase in mTq2 or mRuby3 fluorescence using a microplate reader (ClarioStar, BMG LabTech).

Determining G protein transcript levels in the 10 base GPCR-Gα reporter strains. The 10 base GPCR-Gα reporter strains were individually seeded into pH 5.0 Synthetic Complete Low Fluorescence Screening Media (SCD LoFo), grown for several hours, and back-diluted in pH 7.0 of the same medium to achieve an OD₆₀₀ between 1 and 2 after overnight growth. The next morning, these cultures were back-diluted again into SCD LoFo pH 7.0 and grown to an OD₆₀₀ of 1.0. The procedure ensured that the yeast cells were in log phase for multiple divisions before harvesting their mRNA. Cells were then pelleted and frozen at −80° C. for later processing using a Zymolase enzyme (Zymo Research #E1004) to digest the cell wall (37° C. for 1 hour), YeaStar high purity RNA extraction column kit (Zymo #R1002), and DNasel enzyme treatment (Zymo #E1010) to digest unwanted genomic DNA. The resultant RNA samples were serial diluted to a concentration of 5 ng/uL as confirmed by NanoDrop quantitation. mRNA quantification using one step, gene-specific qRT-PCR was performed on a Bio-Rad CFX384 with a SYBR Green 1-step kit (Bio-Rad SYBR Green 2× iTaq #1725151 with Bio-Rad iScript #L002630) and analyzed with Bio-Rad Maestro qPCR software, with two housekeeper genes for normalization, ALG9 and TAF10. One primer set amplified the native and all humanized yeast Gα chimeras. High primer efficiencies above 90% with an R² value of 97.8 or higher were confirmed on a linear template standard curve from 7 pg/μL to 70 ng/μL. For each primer set, controls without template confirmed a lack of primer-dimer products and controls without the reverse-transcriptase enzyme confirmed a lack of genomic DNA amplification. Each qRT-PCR reaction was 7.125 μL with 11 ng of template (1.54 ng/μL) with each primer at 300 nM.

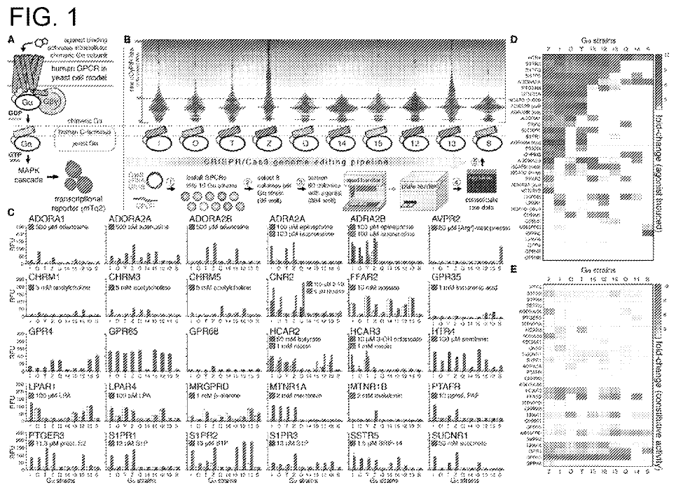

dCyFIRscreen protocol. Individual Gα reporter strains were grown in SCD LoFo pH=7.0 at 30° C. to an OD=1.0 in a 2.0 mL 96-well DeepWell block (Greiner; 780271-FD). Cells were normalized to an OD of 0.1 in SCD LoFo pH=7.0 using a Biomek NXp liquid-handling robot. 10× ligand/vehicle stocks were prepared (see Table 2) and 4 μL were distributed to each well of a 384-well plate (Greiner; 781096) in quadruplicate using a Biomek NXp. 36 μL normalized cells were distributed to each well containing the appropriate 10× ligand/vehicle. Plates were sealed with a breathable cover (Diversified Biotech; BERM-2000) and incubated at 30° C. Fluorescence readings were collected after 18 hours using a plate reader (ClarioStar, BMG LabTech, Offenburg, Germany) (bottom read, 10 flashes/well, excitation filter: 430-10 nm, dichroic filter: LP 458 nm, emission filter: 482-16 nm, gain=1300 (1500 for FIG. 1B.)). Absorbance readings were also collected after 18 hours using the same instrument (22 flashes/well, excitation filter: 600 nm).

dCyFIRplex protocol. Control/tracer strain(s) (DI P1 mTq2, individual Gα reporter strains lacking an integrated receptor, DI P1 mRuby3, and the 300 GPCR-Gα reporter strains were grown in SCD LoFo pH =5.0 to saturation in individual wells of a 2.0 mL 96-well DeepWell block (Greiner; 780271-FD). The 10 GPCR-Gα reporter strains for a single receptor were then consolidated in growth-normalized amounts into single wells of a DeepWell block using a Biomek NXp (each well is comprised of one unique receptor in all 10 Gα reporter strains). Each receptor-consolidated well and control well was grown to mid-log phase in SCD LoFo pH=7.0 then further consolidated into a single tube (300-plex; 30 receptors, 300 Gα strains) in growth-normalized amounts (this consolidation was performed three times for each sorting procedure {n=3}). 10× ligand/vehicle stocks were added to individual wells of a DeepWell block. The consolidated 300-plex or control strains were then added to each well and grown in the presence of ligand/vehicle overnight, so that each culture would reach an OD=4.0 before dCyFIRplex profiling. Samples were washed with sterile ddH₂O and normalized to an OD=2.0 in SCD LoFo pH=7.0. Tracer cells were added to each sample at a 1:301 ratio. The final mixture was then transferred into a glass sample tube (USA Scientific; 1450-2810) and used for cell sorting. A BD FACSAria-II cell sorter was used for all dCyFIRplex experiments to assess mTq2 (405 nm excitation, 450/50 nm emission) and mRuby3 fluorescence (535 nm excitation, 610/20 nm emission). A gating strategy was set using the three control samples (DI P1 mTq2, individual Gα reporter strains lacking an integrated receptor, and DI P1 mRuby3) such that tracer cells and any cell expressing mTq2 was sorted into a 14 mL collection tube (USA Scientific; 1485-2810) containing 500 μL YPD. Samples treated with water or 500 μM adenosine (well-characterized using dCyFIRscreen, inexpensive, and water-soluble) were used to build a standard curve measuring total events in the mRuby3 and mTq2 positive gates. The standard curve from a water-treated 300-plex was used to determine the number of tracer events that would correspond to 15,000 events in the mTq2 gate for a water-treated 300-plex. Each sample was sorted until the standardized tracer count was reached. Sorted cells were enriched by outgrowth in 5 mL YPD at 30° C. with shaking (200 rpm) for 18 hours. Cells were harvested by centrifugation at 3,000×g for 5 min, and resuspended in 1 mL ddH₂O. Cells were either processed immediately for qPCR deconvolution or frozen in 100 μL aliquots for storage at −20° C. The set of samples comparatively deconvoluted by qPCR and NanoString methods were derived from aliquots of the same dCyFIRplex experiments.

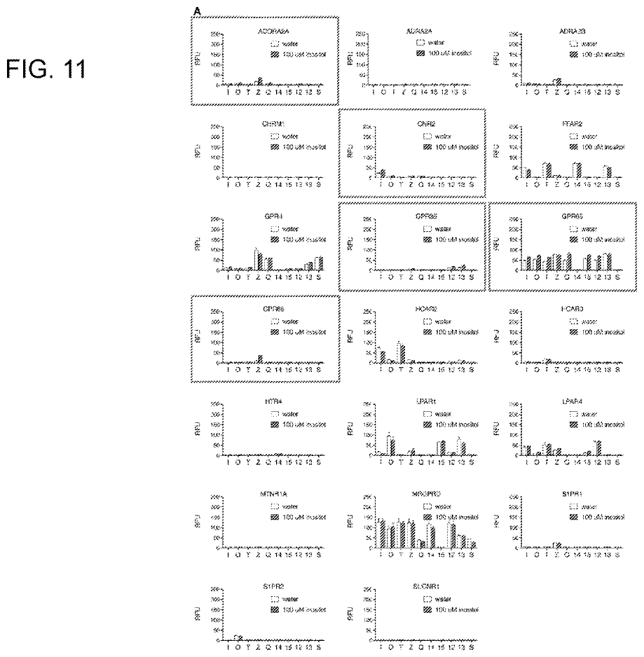

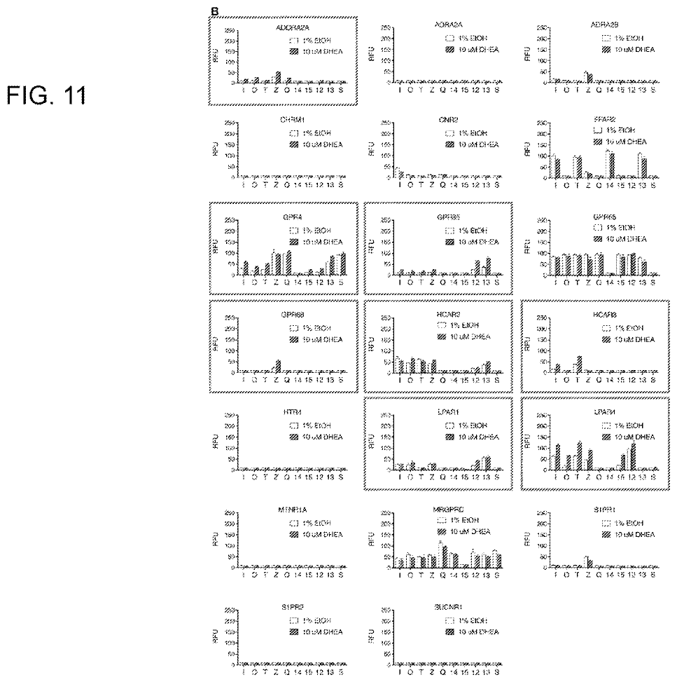

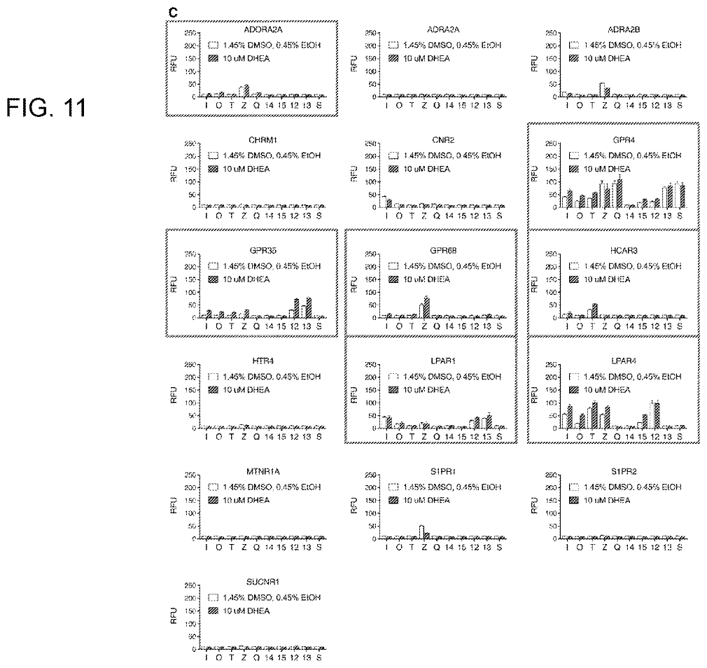

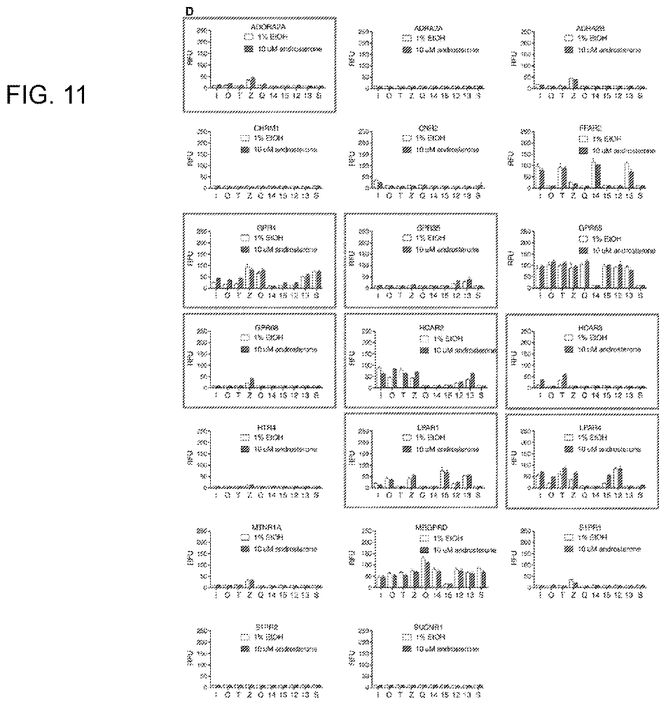

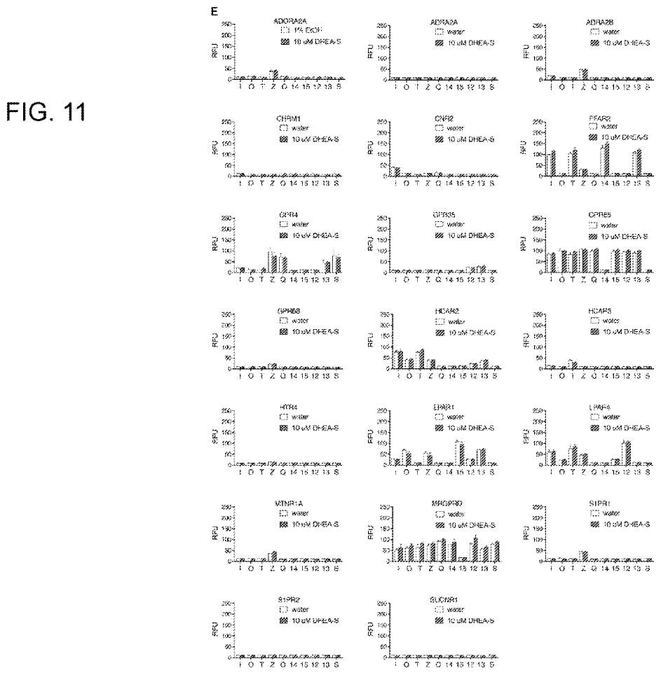

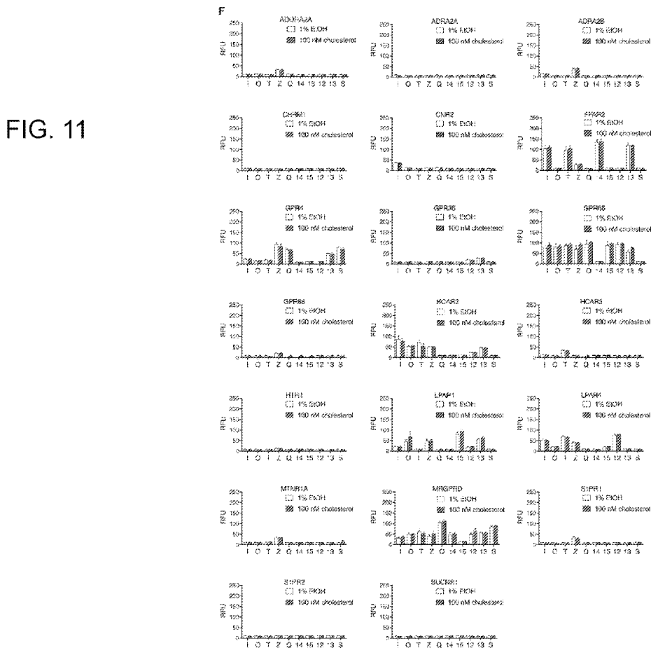

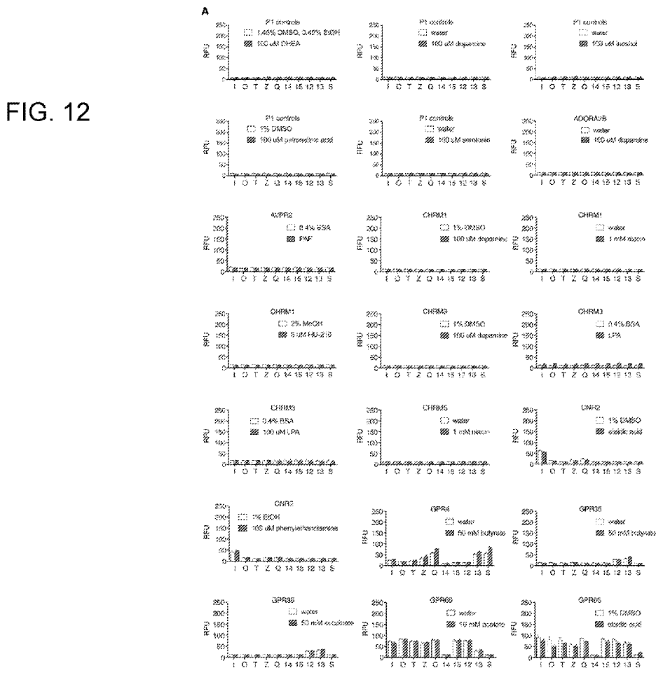

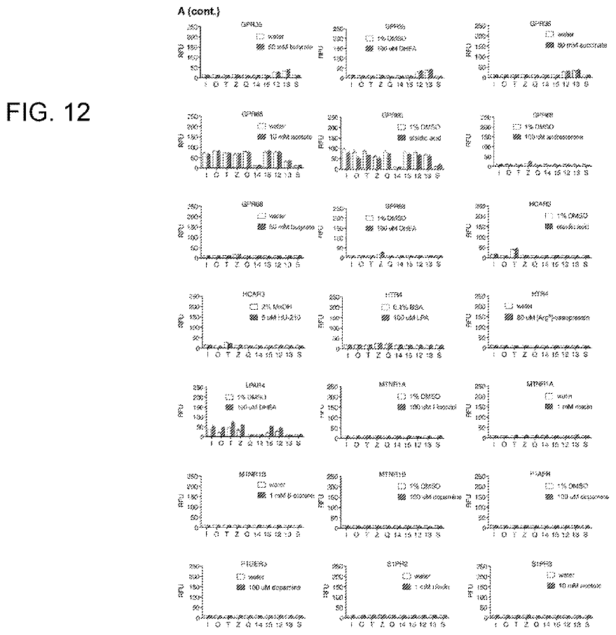

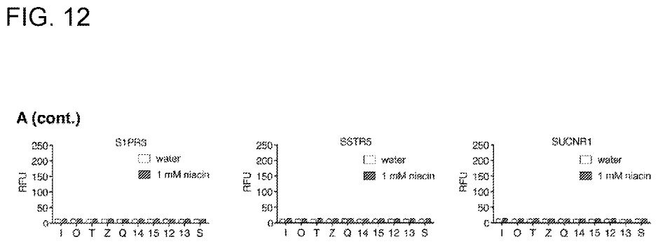

Over the course of our dCyFIRplex profiling, the relationship between the magnitude of ACq values and validated ligand hits were characterized. In the dCyFIRplex datasets, ACq values for most confirmed ligand hits varied between 1 and 8. These log₂ values correspond to a dynamic ΔCq range between 2-fold (2¹) and 256-fold (2⁸). To assess the possibility of ligand hits having ΔCq values between zero and 1, follow-up experiments were performed for 121 select instances in which ΔCq values <1 (presented in FIGS. 11 and 12). Most ΔCq values <1 corresponded to true negatives. These extensive control experiments also helped that agonist-responses from constitutively-active and very weakly-responding receptors give smaller ΔCq values, making it more challenging to detect such hits. Examples include CNR2, LPAR4, FFAR2, MRGPRD, and DHEA positive allosteric modulation of GPR35.

Extraction of genomic DNA for qPCR deconvolution. Yeast sample aliquots (100 μL, see “dCyFIRplex protocol”) were harvested by centrifugation at 8,000×g for 1 minute, resuspended in 200 μL of genomic DNA lysis buffer (200 mM lithium acetate, 1% SDS), and incubated at 70° C. for 10 minutes. Genomic DNA was collected by adding 600 μL of 100% ethanol to each tube of lysed cells and centrifugation at 13,000×g for 5 minutes. Supernatant was then removed and the pellet of genomic DNA was washed with 600 μL 70% ethanol followed by centrifugation at 13,000×g for 5 minutes. The resultant pellet of genomic DNA was dried at 70° C. for 10 minutes before a final resuspension in 50 μL nuclease-free H₂O. Genomic DNA was normalized to a final concentration of 10 ng/μL and used as the template for qPCR deconvolution.

qPCR primer design for dCyFIRplex deconvolution. There were several challenges associated with developing qPCR primers with the specificity and performance necessary to deconvolute complex gene mixtures. These primers must bind only one gene sequence in the mixture, avoid non-specific binding to background genomic DNA, produce the desired amplicon size with an optimal melting temperature, and lack the propensity to form secondary structures (e.g. hairpins), primer-dimers, and primer-heterodimers. To address these issues, a Python program was created that utilized the Primer3 module, a Python-specific application programming interface that provides accessibility to the open source primer design software package Primer3 (www.primer3.org). Using this program, forward primers were designed targeting specific C-terminal sequences in each of the 30 GPCRs in our exploratory panel. These forward primers, when combined with a universal reverse primer (targeting the CYC lb terminator in the X-2 landing pad), were designed to produce amplicons between 111 and 200 bp, an ideal size for qPCR analysis. Using this in silico design process, hundreds of forward qPCR primers for each GPCR using the following Primer3 settings were generated and tested:

PRIMER_OPT_SIZE: 20,

PRIMER_MIN_SIZE: 18,

PRIMER_MAX_SIZE: 22,

PRIMER_OPT_TM: 58,

PRIMER_MIN_TM: 52,

PRIMER_MAX_TM: 60,

PRIMER_MIN_GC: 20,

PRIMER_MAX_GC: 80,

PRIMER_GC_CLAMP: 1,

PRIMER_MAX_POLY_X: 6,

PRIMER_SALT_MONOVALENT: 50.0,

PRIMER_DNA_CONC: 50.0,

PRIMER_THERMODYNAMIC_ALIGNMENT: 1,

PRIMER_MAX_SELF_ANY_TH: 47.0,

PRIMER_MAX_SELF_END_TH: 47.0,

PRIMER_PAIR_MAX_COMPL_ANY_TH: 47.0,

PRIMER_PAIR_MAX_COMPL_END_TH: 47.0,

and PRIMER_PRODUCT_SIZE_RANGE: [111, 200].

The resultant set of primer candidates for each GPCR were ranked from best to worst by their Primer3 scores and assessed for uniqueness via sequential BLAST queries against locally built BLAST databases for 1) the updated release of S. cerevisiae BY4741 genome (BY4741_Toronto_2012) available via www.yeastgenome.org (Cherry, et al., (2012) Nucleic Acids Res 40, D700-705) and 2) the set of GPCR)sequences comprising the Presto-TANGO library (Kroeze, et al., (2015) Nat Struct Mol Biol 22, 362-369). After BLAST filtering, the top 8 primer designs for each GPCR were ordered from ThermoFisher and experimentally validated. In almost all cases, the primer designs met the specificity standards. However, only the top primer design was selected for thedeconvolution procedure.

qPCR dCyFlRplex deconvolution. To achieve an accurate, reproducible, and scalable qPCR deconvolution procedure, all processes were automated using our Biomek NXp liquid-handling robot programmed using in-house Python code. The following procedure describes the process for deconvoluting a 300-plex genomic DNA sample from a single dCyFIRplex experiment. A reaction master mix was created by combining the necessary volumes of qPCR master mix (Bio-Rad Cat. #1725124), universal reverse primer, and template (i.e., the pool of extracted gDNA from a dCyFIRplex profile). In 384-well format, the robot was used to first distribute 3.0 μL of each GPCR forward qPCR primer in duplicate at a concentration of 500 ng/μL. Next, the robot was used to distribute 3.65 μL of reaction master mix to each well, giving a total qPCR reaction volume of 6.65 μL. For 30 receptors in a 300-plex, plus the additional mRuby3 tracer gene, a total of 62 wells were needed. The microplate was removed from the robot deck, centrifuged it for 1 min at 1000×g to consolidate the samples at the bottom of the microplate wells, sealed the microplate with adhesive film (Applied Biosystems Cat. #4311971), and performed the qPCR experiment using a Bio-Rad CFX384.

As described, in each deconvolution run qPCR reactions were done twice for each of the 30 deconvolution primers (and mRuby3 control tracer primer), giving n=2 observations for each GPCR primer. However, in practice, deconvolution was done in triplicate for each agonist treatment using three independent builds of the 300-plex. As a result, there was n=6 observations for each GPCR primer in the deconvolution run. Therefore, all Cq and ACq values reported in this work represent an average of n=6 deconvolution runs, with error bars representing SEM of these values. All Cq values were quantified using the Bio-Rad Maestro qPCR software.

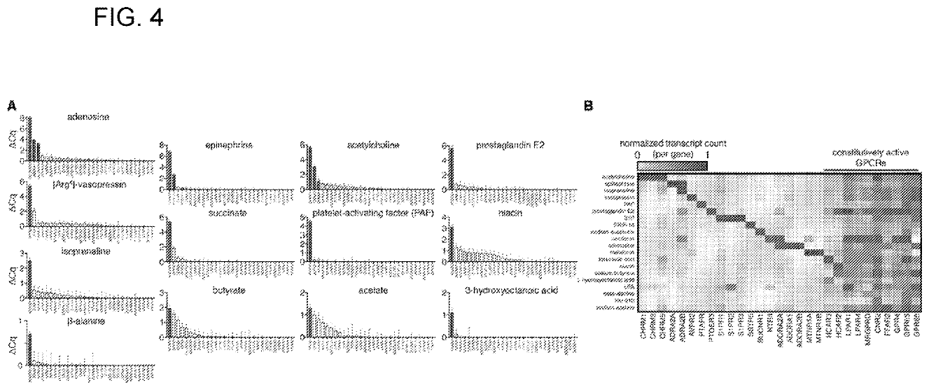

NanoString dCyFIRplex deconvolution. In addition to the qPCR-based deconvolution method, the same 300-plex samples were analyzed and presented in FIGS. 3F and 4A using an orthogonal approach known as NanoString (NanoString Technologies, Seattle, Wash.). Using NanoString, the number of mRNA transcripts can be counted for a given gene using sequence-specific RNA hybridization probes covalently modified with proprietary fluorescent barcodes. Working with NanoString and Integrated DNA technologies as part of NanoString's proof-of-principle program, probes were designed for the panel of 30 GPCR genes and the mRuby3 tracer gene. The total RNA was then purified from the collection of agonist-treated 300-plex samples using an RNA extraction kit (Zymo #R1002). These RNA samples were normalized to a concentration of 45 ng/μL and 45.0 μL volumes were submitted on ice to NanoString. Samples were run in duplicate on the nCounter Sprint Profiler using 100 ng of RNA, then again using 400 ng of RNA, giving a total of n=4 observations for each GPCR transcript.n Qualitatively, both RNA amounts gave the same result; in FIG. 4B the data only for the 400-ng run was presented. To generate the heat map of normalized transcript counts presented in FIG. 4B, the NanoString data was processed in four steps. In step one, data were background subtracted using a minimum value of 20 transcript counts. In step two, data were normalized across the set of GPCRs using the ratio of mRuby3 and GPR4 transcript counts. In step three, duplicate transcript counts for each GPCR were averaged to give a single transcript count value. And in step four, the average transcript count values were normalized across all agonist treatments for a given GPCR. As a result, the agonist responses for each GPCR in the heat map are ranked from zero to 1. Because the NanoString data was collected through the proof-of-principle program, the sample number was limited to the set of receptors and ligands presented in FIG. 4B. Consequently, the only receptor/ligand combinations missing from the set are GPR35 with kynurenic acid and CNR2 with 2-AG and HU-210.

Microscopy. Two microscopy approaches were used in the Examples.

Fluorescence microscopy. Consolidated pools of GPCR-Gα strains treated with metabolites were grown in 384-well plates (see “dCyFIRscreen protocol” above) for 18 hours to an OD˜1.0-2.0. Brightfield and fluorescence images (445/20 nm excitation, 510/40 nm emission, 455 nm longpass dichroic) were captured at 20× magnification using a fluorescence microscope (Echo Revolve, San Diego, USA).

Confocal microscopy. Cells were grown to mid-log phase in SCD LoFo pH 7.0 media. Cells were harvested by centrifugation, washed with sterile H₂O and concentrated to an OD=20 in SCD LoFo pH 7.0 media. 2 μL cells were added to an agar pad (SCD LoFo pH 7.0 media, 15% agar) on a 75×25×1 mm microscope slide (VWR Cat. #16004-422) and covered with a 22×22 mm no. 1.5 glass cover slip (VWR Cat. #48366-227). Brightfield and fluorescence images were captured at 63× magnification under oil immersion on a confocal microscope (LSM800, Carl Zeiss, Jena, Germany). Fluorescence images for mTq2 (433 nm excitation, 475 nm emission) and mRuby3 (587 nm excitation, 610 nm emission) (FIG. 3B) are depicted as maximum intensity projections (MIPs) of a Z-stack composed of 3-7 slices of 1-3 μm. MIP and image overlay for brightfield and confocal images were processed using the Zeiss Zen software.

Ligands and metabolite library. All ligands were purchased from Sigma, Cayman, Tocris, and Avanti. The library of 320 endogenous human metabolites was purchased from MedChem Express (HY-L030). A table describing the source and composition of each ligand is available in the Key Resource Table. Lipid stocks in organic solvent were prepared using the general protocol from Avanti.

Titration Analyses. All titration curves were analyzed using Prism software and the pharmacological fitting function log(agonist) vs. response—Variable slope (four parameters) (GraphPad Software, San Diego, USA).

### ii. dCyFIRscreen Profiling for Metabolite Ligand Discovery

A high-throughput CRISPR/Cas9 genome-editing pipeline for engineering, screening, and validating thousands of yeast reporter strains that were individually barcoded with genome-integrated human GPCRs was created (FIG. 1). We selected the yeast model system Saccharomyces cerevisiae for our strain designs because it has an isolated GPCR pheromone pathway (Bardwell, L. (2005). Peptides 26, 339-350; Wang and Dohlman (2004). Science 306, 1508-1509) that can be adapted to study individual human GPCRs (Dowell and Brown, A. J. (2002) Receptors Channels 8, 343-352; Dowell and Brown (2009) Methods Mol Biol 552, 213-229). The simplified schematic of the humanized pheromone pathway shown in FIG. 1A illustrates how the activation of a human GPCR was coupled to pheromone signaling to build the GPCR-Gα reporter strains (for further details see FIG. 2). Briefly, in a strain lacking the endogenous yeast GPCR (Ste2) a human GPCR was constitutively expressed from a safe harbor locus located in chromosome X, known as X-2 (Ronda et al., (2015). Microb Cell Fact 14, 97). The human GPCR was then stimulated with agonist to activate a chimeric Gα subunit, comprising the endogenous yeast Gα with a humanized C-terminus. Activation of the Gα chimera triggers a MAP kinase signaling cascade that drives the expression of a bright fluorescent transcriptional reporter, mTurquoise2 (mTq2) (Goedhart et al., (2012) Nat Commun 3, 751) installed in place of the pheromone-induced FIG1 gene (FIG. 2).

All 16 human Gα genes can be represented by 10 degenerate C-termini (FIG. 2). As such, 10 versions of the base Gα reporter strains were built. Using the CRISPR pipeline (FIG. 1B), an exploratory panel of 30 human GPCRs was installed into the 10 Gα reporter strains, generating a total of 300 new yeast strains representing all 300 possible GPCR-Gα coupling combinations. Genome engineering with such speed and scale was possible because CRISPR editing in yeast is extremely efficient relative to mammalian cells DiCarlo, et al, (2013) Nucleic Acids Res 41, 4336-4343; Laughery, et al., (2015) Yeast 32, 711-720; Ronda et al., (2015). Microb Cell Fact 14, 97). Although GPCR genome-integration efficiencies >80% was typically achieved, the occasional CRISPR failures were addressed by screening 80 candidate colonies (8 for each GPCR-Gα combination) against one or more known agonists, or no agonist as in the case of constitutively active receptors such as GPR4, GPR65, and GPR68. Using this procedure, a remarkable number of yeast colonies were screened, collecting more than 5,000 raw fluorescence data points for the panel of 30 exploratory receptors. As shown in FIG. 1B, in this initial CRISPR screening step GPCR-Gα reporter strains that signaled strongly (>80 mTq2 relative fluorescence units, RFUs), weakly (30-80 mTq2 RFUs), and not at all (<30 mTq2 RFUs) were observed.

Next the library of 300 GPCR-Gα reporter strains was finalized in preparation for ligand discovery. First, each of the 30 sets of 80 candidate GPCR-Gα reporter strains were screened to PCR-verify the presence of genome-integrated receptors. Then, a single PCR-confirmed hit was selected and stocked for each GPCR-Gα reporter strain (10 Gα strains per GPCR). In the case of constitutively active and agonist-inducible GPCR-Gα reporter strains, the best performing (i.e. brightest) PCR-verified colonies were selected and stocked. The result was a library of 300 GPCR-Gα reporter strains. Importantly, each new reporter strain was barcoded with a single human GPCR and Gα chimera. Prior to developing this multiplexing capability, the GPCR-Gα strain library was validated via an extensive agonist rescreening campaign. For this, all 300 GPCR-Gα strains were analyzed in technical quadruplicate for agonism and constitutive activity in 384-well plate format using an approach calledl dCyFIRscreen (FIG. 1C). From this screen a remarkable 24 of 30 GPCRs (80%) coupled to Gα_{z} (FIG. 1D and 1E), and that 15 of 30 receptors (50%) exhibited some degree of constitutive activity (FIG. 1E).

### iii. dCyFIRplex Profiling for Metabolite Ligand Discovery

Following the dCyFIRscreen (FIG. 1C), which enabled massively parallel screening, dCyFIRplex was developed (FIG. 3), an approach that enabled a single agonist to be queried against a consolidated pool comprising the exploratory GPCR-Gα strain library (i.e. a 300-plex). Using the dCyFIRplex method, agonist-induced mTq2 expression with single-cell was monitored, single-receptor resolution using fluorescence activated cell sorting (FACS) to collect an active pool of signaling GPCR-Gα strains (FIGS. 3A-D) was monitored. The receptor barcode(s) present in the active pool was identified using both automated quantitative PCR and NanoString deconvolution (FIG. 3A). To assemble the screening pools, growth-normalized GPCR-Gα reporter strains were combined in equal parts, and incorporated a tracer strain that constitutively expressed the red fluorescent protein, mRuby3 (FIGS. 3A-D) (Bajar et al., (2016). Sci Rep 6, 20889). The purpose of including the tracer strain was to enable the comparison of different dCyFIRplex runs (FIG. 3C), and to empirically determine the optimal duration of our sorting procedure (FIG. 3D).

The probabilistic character of the dCyFIRplex method is illustrated in FIG. 3, panels B, C, and D. Adding the tracer strain to the 300-plex results in a 1 in 301 chance of a tracer cell-sorting event. However, the chance of a cell-sorting event for a given GPCR depended on its number of active GPCR-Gα strains in the multiplex. For example, the melatonin receptor MTNR1A, which signaled in 4 GPCR-Gα reporter strains (FIG. 1C), has an expected cell-sorting probability of 4 in 301. Confocal images of untreated samples captured solitary tracer cells surrounded by reporter cells comprising the GPCR-Gα strain pool (FIG. 3B). However, constitutively active GPCR-Gα cells were occasionally observed as well (see FIG. 3B, lower right corner of BSA vehicle control). Confocal images taken after agonist treatment showed solitary tracer cells now surrounded by an increased number of active, cyan fluorescent, GPCR-Gα strains (FIG. 3B). These active strains tended to form filamentous arrays, a natural process triggered by activation of the pheromone pathway (Erdman and Snyder (2001) Genetics 159, 919-928). In liquid culture, these cell clusters did not interfere with FACS.

As shown in FIGS. 3C and 3D, the FACS gates were designed to discern tracer, active, and inactive cell pools. To test this gating strategy, the response of the 300-plex to representative peptide (SRIF-14), small-molecule (melatonin), and lipid (sphingosine-1-phosphate, SIP) agonists that activate one (SSTR5), two (MTNR1A and MTNR1B), and three (S1PR1, S1PR2, S1PR3) receptors, respectively were studied (FIG. 3C). Furthermore, the robustness of the gating strategy with panels of negative (the 10 base Gα reporter strains lacking GPCRs) and positive controls (two strains that constitutively expressed the mRuby3 tracer or mTq2 reporter) (FIG. 3D) were extensively tested. 3-5 k tracer counts were used to collect an active pool of 20-25 k sorting events (FIG. 3D). As shown in FIG. 3C, only a small fraction of cell-sorting events for the 300-plex corresponded to tracer (0.4, 0.5, and 0.4%) and active (1.5, 1.0, 1.8%) cell pools following agonist treatments.

### iv. New Interactions for Known Metabolite Ligands With Prototypic and Dark GPCRs

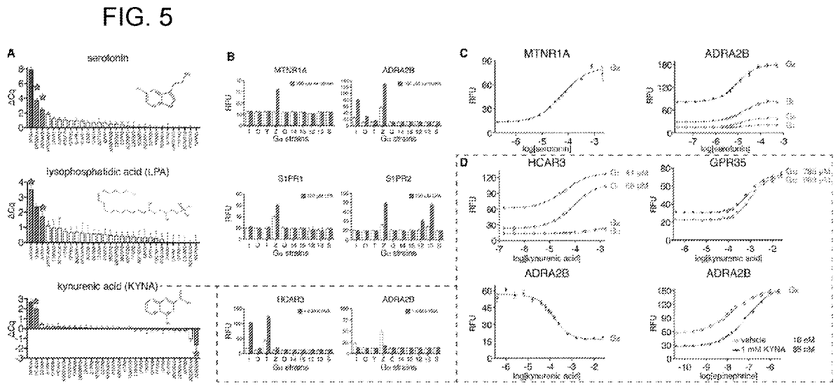

As proof-of-principle for dCyFIRplex sorting and deconvolution, the ability to identify GPCR gene(s) in active pools from a 20-plex of SUCNR1 and HTR4 reporter strains was evaluated (FIG. 3E). Treatment of the 20-plex with succinate and serotonin resulted in active pools that exclusively contained barcoded gene sequences for the receptors SUCNR1 or HTR4, respectively. Next all 300 GPCR-Gα reporter strains were included. Representative results for the 300-plex treated with agonists SRIF-14 (SSTR5), melatonin (MTNR1A and MTNR1B), and S1P (S1PR1, S1PR2, S1PR3) are shown in FIG. 3F. As anticipated, each agonist treatment resulted in active pools exclusively containing the expected receptor gene(s) (FIG. 3F). To demonstrate robustness, the dCyFIRplex procedure was repeated for the full set of agonists (FIGS. 4A-B), achieving a significant advance in the field. As anticipated, the multiplex format led to the discovery of new interactions for known metabolite agonists (FIG. 5A), including interactions of lysophosphatidic acid (LPA) with S1PR2, serotonin with MTNR1A, and the metabolite kynurenic acid (KYNA) with both ADRA2B and remarkably the dark receptor HCAR3.

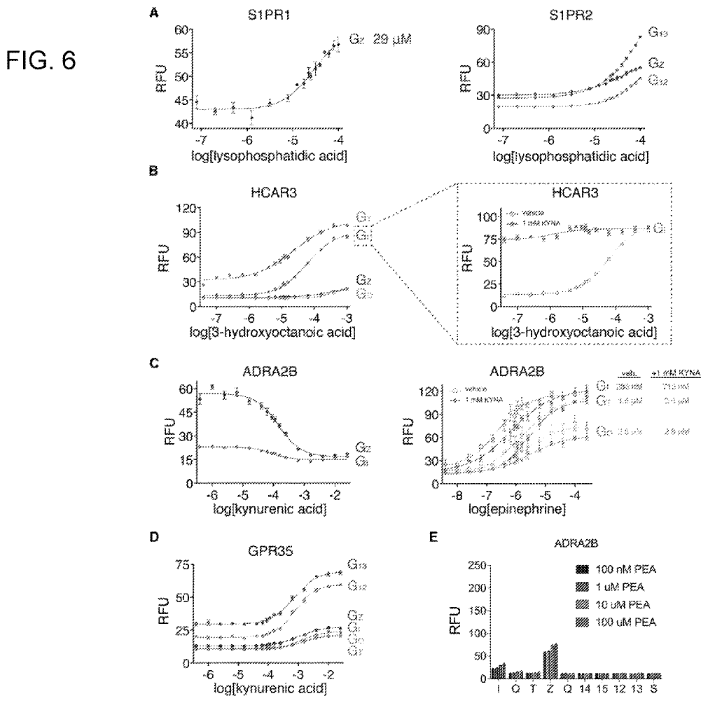

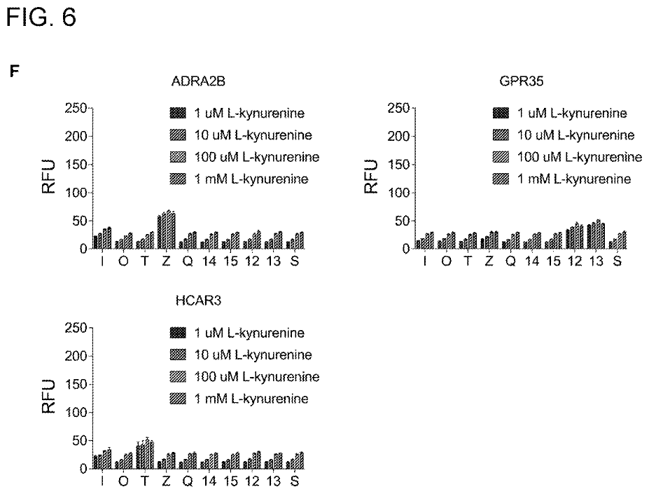

As shown in FIG. 5B, the dCyFIRplex hits in FIG. 5A were confirmed by dCyFIRscreen profiling each new GPCR-agonist interaction. Interestingly, the Gα coupling patterns elicited by these new interactions differed from the coupling preferences of MTNR1A and S1PR2 for their known agonists (FIG. 1C). In only one case of dCyFIRplex profiling (using KYNA) was a reduction observed in signaling upon ligand treatment (ADRA2B, FIG. 5A), suggesting that KYNA was either an inverse agonist or endogenous negative allosteric modulator of ADRA2B. To investigate these possibilities, and fully validate the set of discoveries, titrations were performed. These experiments quantified micromolar interactions of serotonin with MTNR1A and ADRA2B (FIG. 5C), KYNA with HCAR3 and ADRA2B (FIG. 5D), and LPA with S1PR2 (FIG. 6). Additional titrations of HCAR3 and ADRA2B with known ligands (3-hydroxyoctanoic acid and epinephrine) in the presence of KYNA demonstrated that KYNA is an orthosteric agonist of HCAR3 (FIG. 6) and a negative allosteric modulator of ADRA2B (FIG. 5D). Lastly, head-to-head KYNA titrations of GPR35 and HCAR3 led to the finding that KYNA has an 18-fold higher affinity for HCAR3 (EC₅₀ of 41 μM) compared to its known target, GPR35 (EC₅₀ of 795 μM) (FIG. 5D).

### v. dCyFIR Profiling of 320 Metabolites Identifies New GPCR Agonists and Allosteric Modulators

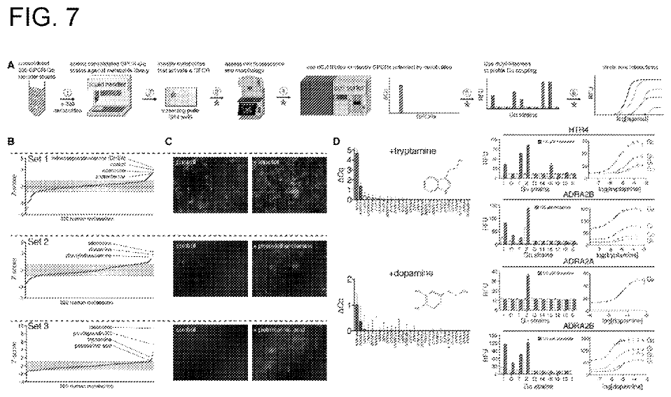

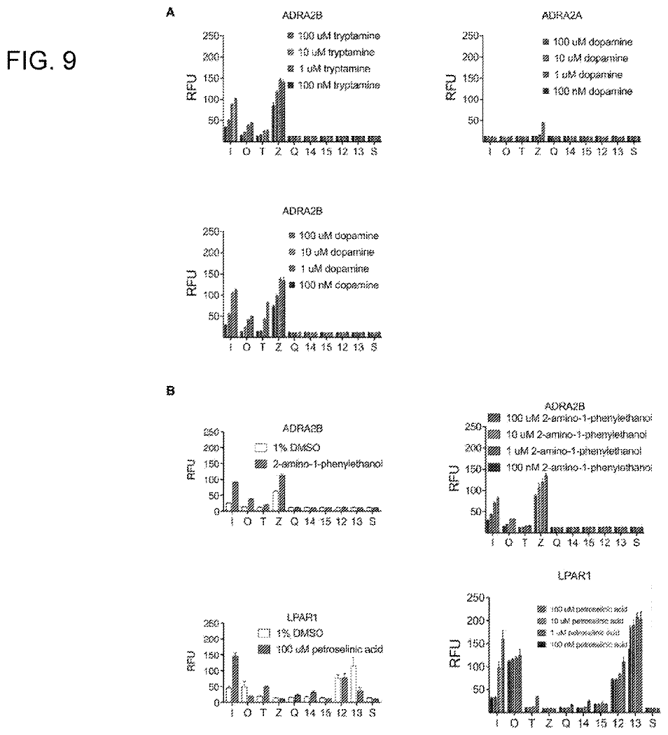

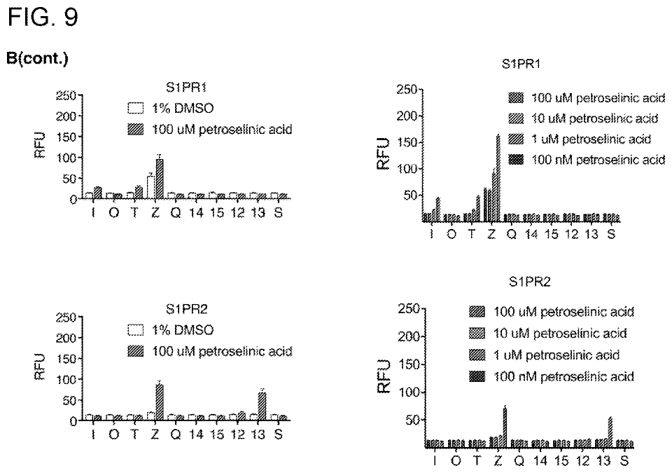

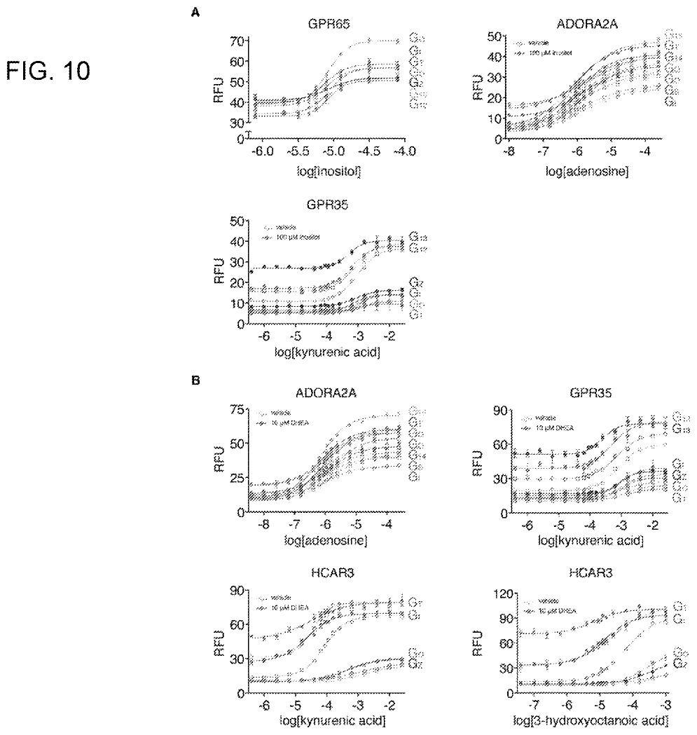

Next, the 300-plex of GPCR-Gα reporter strains were profiled against a library of 320 endogenous human metabolites. As illustrated in the schematic of the discovery pipeline (FIG. 7A), experiments using both dCyFIR profiling modes (dCyFIRscreen and dCyFIRplex) in a 6-step procedure were performed. In step one, GPCR-Gα reporter strains were consolidated in equal parts to create a multiplex culture of many GPCR-Gα combinations. In 384-well plate format, 36 μL of this mixture was combined with 4 μL of each metabolite in duplicate at a concentration of 100 μM. In steps two and three, the mTq2 fluorescence of the samples were measured, calculated sample Z-scores (FIG. 7B), and used fluorescence microscopy to examine microplate wells corresponding to Z-score hits (FIG. 7C). Following hit picking, each potential metabolite ligand using dCyFIRplex profiling was interrogated to identify responsive receptor(s), dCyFIRscreen profiling to assess Gα coupling(s), and titrations in coarse (4-point) and detailed (16-point) formats to estimate or quantify apparent ECso value(s) (FIG. 7A steps 4-6, FIG. 7D, and FIGS. 9 and 10).

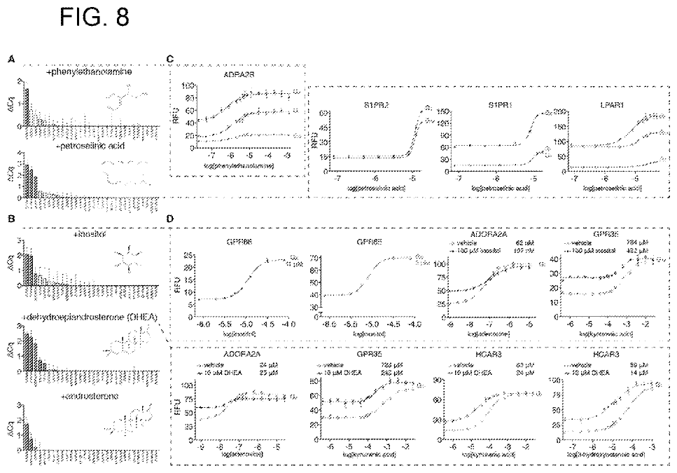

For the initial dCyFIRscreen profiling experiments the 300-plex were divided into three sets (FIG. 5B). Set 1 included 11 receptors (110-plex) that generally exhibited higher levels of constitutive activity. Such receptors are advantageous because they can be used to screen for a broad spectrum of pharmacological effects: agonism, inverse agonism, and positive and negative allosteric modulation. In contrast, receptors lacking constitutive activity, such as most of the receptors included in sets 2 (100-plex) and 3 (90-plex), can be screened only for agonism initially. However, new agonists that are identified for such receptors can be used as pharmacological tools to search for antagonists and allosteric modulators. To identify new metabolite ligands for our 30-receptor panel, each receptor subset was screened in technical duplicate against the metabolite library and selected hits for follow-up studies as having a Z-score>1 (FIG. 7B) and fluorescence microscopy images having marked increases in mTq2 expression, filamentous arrays, and shmooing (FIG. 7C). As anticipated, this screen identified known metabolite agonists for receptors in our panel such as adenosine, melatonin, and prostaglandin E2 (FIG. 7B), corroborated and advanced the scope of recently reported GPCR-metabolite interactions for tryptamine (Bhattarai, Y., et al., (2018) Cell Host Microbe 23, 775-785 e775) and dopamine (Galinski, S. et al., (2018) Sci Rep 8, 8137) (FIG. 7D), and led to our discovery of several new and unexpected GPCR-metabolite interactions with phenylethanolamine (PEOA), inositol, petroselinic acid, and the steroid metabolites androsterone and dehydroepiandrosterone (DHEA) (FIG. 8A-B). Remarkably, most of these newly discovered metabolite interactions pertained to dark receptors GPR4, GPR65, GPR68, and HCAR3.

FIG. 7D illustrates the procedure for validating metabolite hits using tryptamine and dopamine as examples. In each case, dCyFIRplex profiling was used to identify known (HTR4) and new (ADRA2B) tryptamine-binding GPCRs and known (ADRA2B) and new (ADRA2A) dopamine-binding GPCRs. To confirm these findings and characterize Gα coupling patterns, dCyFIRscreen profiling was used to establish that all 4 GPCR-metabolite interactions signaled through the Gαᵢ family of GPCR-Gα strains. Interestingly, tryptamine also elicited HTR4 signaling through the Gα15 reporter strain, demonstrating the advantage of the approach of testing all 10 possible GPCR-Gα combinations. As shown by the titrations in FIG. 7D, all tryptamine and dopamine interactions we observed had EC₅₀ values in the micromolar range and relative efficacies consistent with their accompanying dCyFIRscreen profiles.

As shown in FIG. 8A, the results for the newly discovered GPCR-metabolite interactions involving PEOA were similar to tryptamine and dopamine. However, the dCyFIRplex profiles for petroselinic acid, inositol, and the steroid metabolites DHEA and androsterone each indicated several potential new GPCR-metabolite interactions. In such cases, dCyFIRscreen profiles were collected for many receptors in the exploratory panel to directly assess ligand specificity (over 150 control dCyFIRscreen profiles and titrations are available in FIGS. 9-12). This process indicated that petroselinic acid was an agonist specific for S1PR1, S1PR2, and LPAR1 (FIG. 8A), and suggested that inositol and the steroid metabolites DHEA and androsterone were broad spectrum positive allosteric modulators (FIG. 8B and FIGS. 10 and 11). To test this possibility, these metabolites were dCyFIRscreen profiled (and DHEA-S and cholesterol controls) against 20 GPCRs (200 GPCR-Gα strains) that included all 15 constitutively active receptors in the exploratory panel (Figure S5). As anticipated, all three metabolites appeared to increase receptor signaling, with inositol modulating 5 receptors (ADORA2A, CNR2, GPR35, GPR65, GPR68) and the structurally similar steroid metabolites DHEA and androsterone modulating 8 receptors (ADORA2A, GPR35, GPR4, GPR68, HCAR2, HCAR3, LPAR1, LPAR4). Interestingly, all three metabolite ligands poly-modulated three receptors (ADORA2A, GPR35, GPR68). To confirm ther findings in pharmacological detail, select constitutively active (GPR65 and GPR68) and agonist responsive (ADORA2A, GPR35, HCAR3) receptors were titrated in the presence of inositol and DHEA (FIG. 8D).

Based on the methods and cells disclosed herein, several new interactions between GPCRs and amino acid metabolites from the tryptophan, phenylalanine, and tyrosine pathways were identified. Three of these metabolites were from the tryptophan pathway and included an inflammation biomarker (KYNA), neurotransmitter produced in the gut and brain (serotonin), and neuromodulator produced in the brain, gut, and found at high concentrations in fermented foods (tryptamine) (Hastings, et al., (2016) Nucleic Acids Res 44, D1214-1219; Wishart et al., 2018 Nucleic Acids Res 46, D608-D617). Although structurally similar, these metabolites exclusively interacted with a wide variety of GPCRs. KYNA activated GPR35 and the dark GPCR HCAR3 and was a negative allosteric modulator of ADRA2B.

Surprisingly, it also found that serotonin activated the melatonin receptor MTNR1A. Although serotonin and melatonin receptors bind similar endogenous molecules, few of these ligands are known to bind receptors from both families (Stauch et al., (2019) Nature 569, 284-288). The discovery that serotonin can activate MTNR1A appears to be an exception to this rule, and may have been overlooked due to the relatively low, yet metabolically relevant affinity of MTNR1A for serotonin (EC₅₀ of 60 μM). Notably, it was not find that serotonin agonized the other melatonin receptor in our set (MTNR1B), nor was it observed that melatonin bound to the serotonin receptor HTR4. Similarly, tryptamine, which is almost identical in structure to serotonin (5-hydroxytryptamine), did not activate either melatonin receptor, but did activate HTR4 and ADRA2B. Recently it has been shown that tryptamine produced in the gut, rather than in the brain, can also activate HTR4 (Bhattara et al., (2018) Cell Host Microbe 23, 775-785 e775). As such, the findings now implicate ADRA2B as an additional regulatory target of tryptamine.

In addition to tryptamine, new GPCR interactions were observed involving two other trace amines, PEOA and phenethylamine (PEA), both phenylalanine metabolites. PEOA and PEA are produced in the brain, where they can act as neuromodulators and neurotransmitters and activate the trace amino-associated receptor 1 (TAAR1), a GPCR also known to bind tryptamine Rutigliano et al., (2017) Front Pharmacol 8, 987; Wainscott et al., (2007) J Pharmacol Exp Ther 320, 475-485). Recently it was shown that PEA is also produced by the gut microbiota and can act as a dopamine receptor agonist (Chen et al., (2019) Cell 177, 1217-1231 e1218). It was found that PEOA and PEA were also nanomolar (FIG. 8A) and micromolar (FIG. 6) agonists of ADRA2B, respectively, and that dopamine, a tyrosine metabolite, was a micromolar agonist of ADRA2A and ADRA2B (FIG. 7D). Given that PEA and tryptamine are produced by gut bacteria and can cross the blood-brain barrier, the findings suggest that the adrenergic receptors ADRA2A and ADRA2B, as well as dopamine receptors, may play defining roles in the neurological circuits of the emerging gut-brain signaling axis.

Gut microbiota can produce a variety of metabolites including short-chain fatty acids, secondary bile acids, and a variety of neurotransmitters (Husted et al., (2017) Cell Metab 25, 777-796; Strandwitz (2018) Brain Res 1693, 128-133). A s mentioned, some of these metabolites, such as PEA and tryptamine, can readily cross the blood-brain barrier and possibly elicit neuromodulatory effects. However, most other microbiome-derived metabolites are excluded from the central nervous system and instead are absorbed into the circulation and distributed throughout the body, where their effects are poorly understood. Two recent studies shed light onto the fate and actions of a few of these bacterially-derived metabolites. In screens of microbiome extracts, Chen et al. identified PEA as a dopamine receptor agonist (Chen et al., (2019) Cell 177, 1217-1231 e1218)). The current results substantiate these findings, and additionally show that PEA is an agonist of another aminergic receptor, ADRA2B. Chen et al. also found that dopamine agonized ADRA2B, which was confirmed by our screen, in addition to our new finding that dopamine agonizes ADRA2A.

In a second gut microbiome study, Colosimo et al. identified niacin, 3-hydroxyoctanoic acid, and phenylpropanoic acid as agonists of HCAR3 (Colosimo et al., 2019). The current results substantiate these findings as well; however, higher HCAR ligand affinities were observed. For example, Colosimo et al. observed a 12.6-fold higher EC₅₀ value for 3-hydroxyoctanoic acid agonism of HCAR3 (304 μM versus 24 μM in this study). This observation, and their high EC₅₀ value for phenylpropanoic acid binding (208 μM), led the authors to speculate that either low binding affinities were inherent to HCAR3, or that the endogenous ligand for HCAR3 has yet to be identified (Colosimo et al., (2019) Cell Host Microbe 26, 273-282 e277). However, the current findings indicate that KYNA activates HCAR3 with a 5.1-fold lower EC₅₀ value than phenylpropanoic acid and 18.6-fold lower EC₅₀ value than for its known target GPR35. These observations indicate that both KYNA and 3-hydroxyoctanoic acid are the highest affinity endogenous ligands known for HCAR3.

The exploratory panel of 30 receptors included members of two evolutionarily related lipid receptor families, LPAR and S1PR. Both of these receptor families are involved in inflammatory responses, fibrosis, and a variety of other disorders (Blaho and Hla (2014) J Lipid Res 55, 1596-1608; Yung et al., (2014) J Lipid Res 55, 1192-1214) . The LPAR and S1PR families have 30-35% sequence identity, and also share similar spatially-conserved residues that determine their respective specificities for LPA and S11³ metabolites (Wang et al, (2001) J Biol Chem 276, 49213-49220). Here, the known cross-activation of LPA for S1PR1 was confirmed and the first report of LPA cross-activation of S1PR2 (FIG. 5A-B). Consistent with the first studies of LPA cross-activation of S1PR1, LPA bound both S1PR1 and S1PR2 had higher EC₅₀ values compared to LPAR1, indicating that S1PR1 and S1PR2 are low affinity receptors for LPA.

Allosteric GPCR ligands bind to locations outside the orthosteric binding site (Thal et al., (2018) Nature 559, 45-53). Such ligands can act as positive/negative allosteric modulators (PAMs/NAMs), either by modulating constitutive activity and/or receptor responses to orthosteric agonists. Pharmacologically, the actions of PAMs and NAMs cause shifts in EC₅₀ values and changes in signaling strength (i.e. efficacy). However, these effects typically occur in combination, giving rise to a variety of classifiable pharmacological binding profiles (Kenakin (2012) Br J Pharmacol 165, 1659-1669). Structural studies over the past decade have identified a variety of endogenous allosteric modulators, such as ions, lipids, amino acids, peptides, and accessory proteins (van der Westhuizen et al, (2015) J Pharmacol Exp Ther 353, 246-26).

The metabolite inositol is a structural isoform of glucose that is used as a dietary supplement, present in many foods, and produced from glucose in the kidneys. It serves as a precursor for a variety of second messengers and is an important component of lipids ((Wishart et al., (2018) Nucleic Acids Res 46, D608-D6178). Here, inositol is shown to be a PAM-agonist of GPR65, GPR68, and GPR35, and a NAM-agonist of ADORA2A (FIG. 8D).

The endogenous steroid metabolites DHEA and its sulfonated form DHEA-S are the most abundant circulating steroid hormones in humans (Rutkowski et al., (2014) Drugs 74, 1195-1207). DHEA is produced in the adrenal glands, gonads, adipose tissue, and brain ((Wishart et al., (2018) Nucleic Acids Res 46, D608-D617), is widely used as a nutritional supplement, and is the indirect precursor to estrogen, testosterone, and other steroid hormones (Sahu et al., (2019) Steroids 153, 108507). While steroids such as DHEA typically exert effects on steroid receptors in the nucleus, the discovery of extranuclear mediators of steroid responses has garnered long-standing interest in the field (Losel and Wehling (2003) Nat Rev Mol Cell Biol 4, 46-56). Here, it was shown that structurally similar steroids DHEA and androsterone are broad-spectrum PAM-agonists of several GPCRs (FIG. 8D and FIG. 11), with both steroid metabolites interacting with three dark receptors (GPR4, GPR68, HCAR3). Interestingly, DHEA is also a PAM-agonist for both GPR35 and HCAR3, the two GPCRs for which had KYNA binding. To assess the specificity of these findings, a large set of control experiments were performed using DHEA-S and cholesterol, the precursor of both steroids and widely recognized broad-spectrum PAM for class A GPCRs ((Thal et al., (2018) Nature 559, 45-53; (van der Westhuizen et al, (2015) J Pharmacol Exp Ther 353, 246-26; Wacker et al., (2017) Cell 170, 414-427) (FIG. 11). Neither of these ligands elicited responses. (FIG. 11). Notably, DHEA is a documented PAM of the NMDA receptor and NAM of the GABAA receptor (Prough et al., (2016) J Mol Endocrinol 56, R139-155). Lastly, these steroid-related discoveries stand as an example of the advantages of the yeast-based platform for human GPCR screening. Because yeast lack cholesterol and have primitive steroid pathways Parks and Casey (1995) Annu Rev Microbiol 49, 95-116), there is little to no steroid interference from the model system or its genetic background in the discovery process.

Two recent studies have illuminated the pharmacotherapeutic potential of GPR68, a dark receptor known for its pH sensing capabilities (Ludwig et al., (2003) Nature 425, 93-98). Huang et al. have recently identified the benzodiazepine drug lorazepam as a non-selective GPR68 PAM (Huang et al., (2015) Nature 527, 477-483) and Foster et al. have identified the first known peptide ligands for GPR68, which also function as PAMs (Foster et al., (2019) Cell 179, 895-908 e821). Here, GPR68 PAMs comprising the endogenous metabolites inositol, DHEA, and androsterone. Given that GPR68 was expressed in the brain and is important for processes such as learning and memory, the findings may help to explain the mechanistic effects of inositol and DHEA in several neuropsychiatric conditions. Beyond GPR68, this is the first report of endogenous metabolite PAMs for three additional dark GPCRs, GPR65 (inositol), GPR4 (DHEA and androsterone), and HCAR3 (DHEA and androsterone).

KYNA is a tryptophan metabolite linked to neuroprotection, depression, schizophrenia, obesity, diabetes, and cancer. Prior to this study, it was only known to target GPR35 (Wang et al, (2006) J Biol Chem 281, 22021-22028). Here, KYNA was a more potent agonist of the dark receptor HCAR3 and acted as a NAM of ADRA2B (FIG. 5D). DHEA was also a PAM of GPR35 and HCAR3, and that inositol was a PAM of GPR35 (FIG. 8D). In the case of HCAR3, DHEA PAM interactions extended to both orthosteric agonists KYNA and 3-hydroxyoctanoic acid (FIGS. 8 and 10), similar to the dual regulation imposed by KYNA and DHEA on the N-methyl-D-aspartate receptor (Mok et al., (2009) Neuropharmacology 57, 242-249).

### Example 2: Mulit-Padded GPCR Reporter Strains

### i. Materials and Methods

Materials. Yeast extract, yeast nitrogen base, peptone, tryptone, and 5-fluoroorotic acid (5-FOA) were purchased from Research Products International (RPI; Mt. Prospect, Ill.). Low-fluorescence yeast nitrogen base used for preparing screening media was purchased from Formedium (Hunstanton, UK). Complete supplement mixture and complete supplement single dropout (without Uracil) mixture were purchased from MP Biomedicals (Solon, Ohio). Screening media was adjusted to desired pH with HCl or KOH and were buffered with potassium phosphate dibasic (Alfa Aesar; Ward Hill, Mass.) and MES hydrate (RPI).

Plasmids. All CRISPR plasmids used in this work were derived from pML104. Four new versions of the pML104 plasmid were made to first install each landing pad into the genome loci X-2, X-3, XI-2, and XII-5. Four additional versions of the pML104 plasmid were then made for targeting DNA payloads to the artificial guide sequences within each CRISPR-addressable landing pad. All plasmids were maintained in the E. coli strain DH5α (New England BioLabs; Ipswich, Mass.) and purified using the EZ Plasmid Miniprep Kit (EZ BioResearch; St. Louis, Mo.). Genes for GPR68 and SSTR5 were sourced from the PRESTO-TANGO plasmid library.

Media, Buffers, and Solutions. The complete list of growth media, buffers, and solutions used in this study can be found in Table 1. Growth media, buffers, and solutions prepared at specified pH values were measured using an Accumet XL150 pH meter (Fisher Scientific; Hampton, N.H.).

CRISPR protocol. Preparing base strains. Base yeast strains were struck from glycerol onto YPD plates and incubated at 30° C. for 1-2 days. Colonies were picked into 5 mL YPD and grown at 30° C. shaking (200 rpm) until an OD₆₀₀ of 0.2−1.0 was reached.

Preparing cells for transformation. Log-phase cultures were centrifuged (3000×g for 3 min), harvested and washed with 5 mL TE. Cells were centrifuged, harvested, washed with 5 mL LiOAc mix, centrifuged again, and resuspended in 200 μL LiOAc mix.

Preparing transformation mixtures. The solution for a single yeast transformation reaction comprised 175 μL PEG mix, 250 ng CRISPR plasmid, 20 μL DNA payload (5-15 μg DNA total), and 5 μL salmon sperm DNA (boiled at 100° C. for 10 min then placed on ice immediately after boiling).

Transformation procedure. 50 μL of prepared cells were added to the transformation mixture described above. Mixtures were briefly vortexed, then incubated at room temperature for 30 minutes, spiked with 12 μL DMSO, vortexed, and incubated at 42° C. for 15 minutes. The mixtures were then centrifuged (5000×g for 1 min) and the harvested cells were resuspended in 200 μL YPD by gently pipetting 5-8 times. The resuspended cells were plated onto SCD-U agar plates and grown at 30° C. for 3 days. This protocol works for well for plating on both large (100 mm petri dishes, plate 100 μL) and small (22 mm 12-well petri dishes, plate 35 μL) agar plates.

Validation and storage of yeast strains. Genomic DNA extraction to confirm payload integration. Transformed colonies were picked into SCD-U liquid medium and grown at 30° C. for 1-2 days. Genomic DNA (gDNA) was then extracted and purified as previously described (15). Briefly, 100 μL resuspended cells were added to a 1.5-mL Eppendorf tube, centrifuged (15,000×g for 3 min), harvested, and resuspended in 100 μL extraction buffer. Cells were then resuspended by vortexing and incubated at 70° C. for 10 minutes. 300 μL 100% EtOH was added to the mixture, which was then vortexed, and centrifuged. The gDNA pellet was washed with 70% EtOH and dried at 70° C. for 10 minutes. The dried gDNA pellet was resuspended in 50 μL nuclease-free H₂O by thorough vortexing and pipetting, centrifuged (15,000×g for 30 s), and 25 μL of supernatant containing the purified gDNA was transferred to a clean 1.5-mL Eppendorf tube. 1 μL of purified gDNA was then used to PCR-verify integration of the desired DNA payload. PCR reactions were resolved on 1% agarose gels and imaged using an Amersham Imager 600 (GE Healthcare Bio-Sciences; Pittsburgh, Pa.).

Removing the CRISPR plasmid by counter-selection. For a given CRISPR reaction, one strain containing the correctly integrated gene was struck from SCD-U liquid medium onto a CSM+5FOA plate and placed at 30° C. until colonies were present (≈2 days).

Yeast glycerol stocks. One colony was picked from a CSM+5FOA plate into 3 mL YPD and grown at 30° C. overnight. Using this culture, gDNA was purified and PCR verified as described in Genomic DNA extraction to confirm payload integration. For a single PCR-verified strain, 15% v/v glycerol stocks were prepared for long-term storage at −80° C.

Landing pad design and integration. Landing pad design. The X-2 landing pad was synthesized in a pMARQ plasmid (Invitrogen; Carlsbad, Calif.), and the X-3, XI-2, and XII-5 landing pads were synthesized as gBlocks (IDT; Coralville, Iowa). All four CRISPR-addressable landing pads contained a unique core sequence (a 32 bp synthetic sequence consisting of a 20 bp unique targeting site (UnTS), 3 bp PAM site, and 9 bp of buffer DNA) flanked by a P_{TEF1} (419 bp) promoter and T_{CYC1b} terminator (242 bp). Additionally, each landing pad cassette was flanked upstream and downstream by 110 bp of homology to the X-2, X-3, XI-2, or XII-5 chromosome loci.

Landing pad integration. DNA payloads were prepared by PCR amplifying the gBlocks described in Landing pad design. Using the CRISPR protocol described above, each DNA payload and its cognate CRISPR plasmid (pML104 X-2, X-3, XI-2, or XII-5) were co-transformed into the desired base yeast strain. Integration of each landing pad was then validated as described in Validation and storage of yeast strains and confirmed via Sanger sequencing (Eurofins Genomics; Louisville, Ky.). Four-padded strains were created by installing the landing pads sequentially in the following order: X-2 (first), XII-5, X-3, and XI-2 (last).

Using the CRISPR-addressable landing pads. Preparing the DNA payload. DNA payloads originating from plasmid sources (i.e. mTq2, pHluorin, mRuby3, GPR68, and SSTR5) were prepared via two rounds of PCR. The first round of PCR amplified the desired gene, while the second round of PCR extended the amplified gene product with 60 bp of homology to the TEF1 promoter and CYC1b terminator. In some case, the 60 bp of TEF1 and CYC1b homology could be provided directly by PCR primers, and introduced in one PCR reaction (Ste2 sourced from the yeast genome and the mNeonGreen and SRIF-14 sourced from gBlocks).

CRISPR-addressable gene integration. DNA payloads were installed into the desired landing pads using the CRISPR protocol described above, and the cognate CRISPR plasmid (i.e. pML104 X-2 UnTS, X-3 UnTS, XI-2 UnTS, or XII-5 UnTS). For strains in which a fluorescent protein gene was integrated, transformant colonies on SCD-U plates were imaged using an Amersham Imager 600 (excitation filters: 460 nm, 520 nm) to identify fluorescent colonies. All integrations were validated using the approach described in Validation and storage of yeast strains.

Fluorescence measurements. A CLARIOstar multimode microplate reader (BMG LabTech; Offenburg, Germany) was used for all microplate-based fluorescence (bottom read; 10 flashes/well; excitation: 430-10 nm, dichroic filter: LP 458 nm, emission filter: 482-16 nm) and absorbance (22 flashes/well; excitation filter: 600 nm) measurements.

Sample preparation. Strains were struck from glycerol onto YPD plate(s) and grown at 30° C. for 1-2 days. Four colonies per strain were picked from YPD into 96-well deep well blocks (Greiner Bio-One; Item #780271-FD) containing 500 μL SCD Screening media was adjusted to pH 6.5 and grown at 30° C. until they reached log-phase growth (OD₆₀₀ₘₙ 0.2-1.5). 200 μL aliquots of cells were transferred to a 96-well plate(s), centrifuged (3000×g for 5 min), harvested, and resuspended in 200 μL screening media adjusted pH 6.0 (pH 7.0 for SSTR5 and SRIF-14 experiments). Resuspended cells were used to prepare 200 μL of normalized cultures in 96-well format having an OD₆₀₀ₘₙ of 0.05 using a Biomek NX^{P} liquid-handling robot. Plates with normalized cultures were covered with porous film (Diversified Biotech; Cat. #BERM-2000), shaken (1200 rpm for 30 s) on a MixMate microplate shaker (Eppendorf; Hamburg, Germany), and incubated at 30° C. for 18 hours.

Data acquisition. All data were collected from four biological replicates (i.e. colonies), the fluorescence of which was measured over a linear dilution series. This data was fit in Prism to generate slope and intercept values that were used to extrapolate mTq2 fluorescence to a standardized OD₆₀₀ₘₙ value of 1.0. Error bars represent the standard error of the fitted slopes. All samples were assayed as 50 μL aliquots in black 384-well clear-bottom plates (Greiner Bio-One; Item #781096).

Confocal microscopy. Colonies were picked into screening media pH 6.0 and grown at 30° C. with shaking until an OD₆₀₀ₘₙ of 1.0 was reached. Cultures were then centrifuged (3000×g for 3 min), harvested, and cells resuspended in 200 μL fresh pH 6.0 media. 2 μL of cells were added to a 75×25×1 mm microscope slide (VWR; Cat. #16004-422) and covered with a 22×22mm no. 1.5 glass cover slip (VWR; Cat. #48366-227). Cells were imaged using an LSM800 confocal microscope (Carl Zeiss; Jena, Germany) at 63× magnification. For a given field, both DIC and fluorescence images were acquired, which were overlaid and further processed using Zeiss's Zen software. Fluorescence images were acquired using excitation lasers of 405 nm (mTq2; 2.00% intensity), 561 nm (mRuby3; 2.00% intensity), 405 and 488 nm (pHluorin; 3.50% and 4.50% intensity, respectively), and 488 nm (mNeonGreen; 0.04% intensity)

### ii. Ste2 Expression From CRISPR-Addressable Landing Pads Rescues Pheromone Signaling

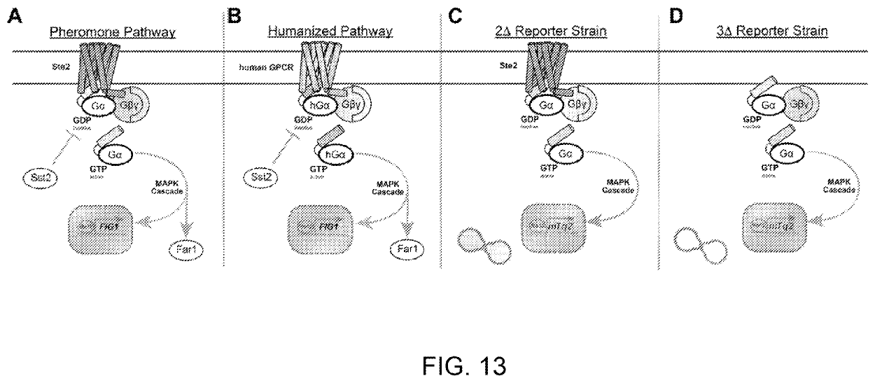

Engineering multi-padded GPCR reporter strains required several base strains as described in the examples above. As shown in FIG. 13, the GTPase-activating protein Sst2 and cell cycle arrest factor Far1 were deleted to create the 2Δ reporter strain. These gene deletions served to sensitize the pheromone pathway (sst2Δ), and prevent cell cycle arrest in response to pathway activation (far1Δ). Additionally, the cyan fluorescent protein mTurquoise2 (mTq2) was installed in place of the dispensable pheromone-responsive gene FIG1. This 2Δ reporter strain served as a reference control for pheromone-induced mTq2 fluorescence because it retains the yeast GPCR Ste2 in its endogenous locus.

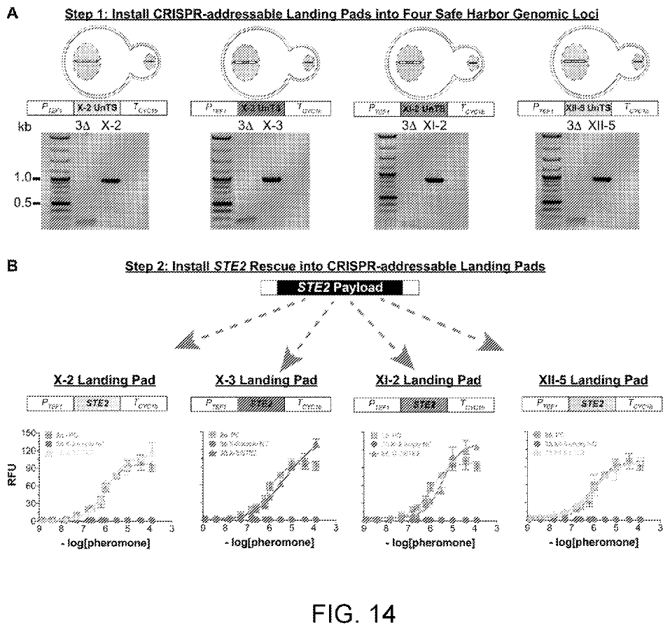

Using the 2Δ reporter strain as a starting point, a series of strains to individually test the functionality of each CRISPR-addressable landing pad were generated. The native yeast GPCR (ste2Δ) was deleted to create the 3Δ reporter strain (FIG. 13D). As shown in FIG. 14A, four single-padded strains were created with CRISPR-addressable landing pads installed at the safe harbor chromosome loci X-2, X-3, XI-2, and XII-5. Each landing pad contained a 20 bp unique targeting sequence (UnTS) and protospacer adjacent motif (PAM) flanked by a TEF1 promoter and CYC1b terminator. To avoid off-target editing, each UnTS was computationally designed to be an artificial 20 bp DNA sequence that did not occur in the yeast genome.

A series of Ste2 rescue experiments were conducted to demonstrate the functionality of each landing pad. This was done by installing the yeast GPCR Ste2 into the CRISPR-addressable landing pad of each single-padded 3Δ reporter strain (FIG. 14B). To test each Ste2 pad installation, pheromone titrations were performed and observed near identical pheromone EC₅₀ values and efficacies for each pad (FIG. 14B). Furthermore, the pheromone EC₅₀ values for the Ste2 rescues were in excellent agreement with the ECso values for Ste2 expressed from its endogenous genome locus in the 2Δ reporter strain. Taken together, these data confirmed the near equivalent functionality of the four X-2, X-3, XI-2, and XII-5 landing pads.

### iii. Engineering Four-Padded GPCR Reporter Strains

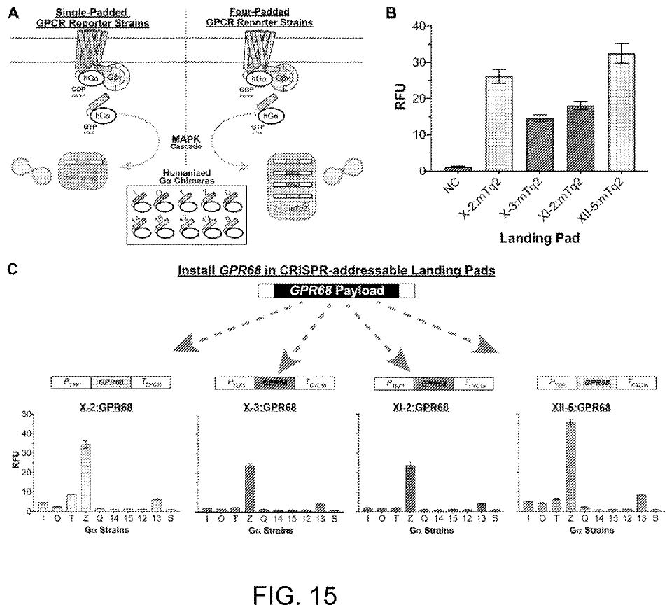

Having validated the performance of each landing pad individually, all four landing pads were combined into a panel of GPCR reporter strains that cover all possible human Gα coupling combinations. As illustrated in FIG. 15A, the strain building process began with a panel of 10 humanized GPCR reporter strains that contained a single CRISPR-addressable landing pad in the X-2 genome locus. In a parallel series of 30 CRISPR genome edits, three additional landing pads X-3, XI-2, and XII-5 into each of the GPCR reporter strains. This process generated 20 intermediate two-padded and three-padded strains, and a final set of 10 new four-padded GPCR reporter strains. The cyan fluorescent protein mTq2 were tested to evaluate the individual performance of the landing pads in one of the four-padded GPCR reporter strains. To do this mTq2 was installed into the four-padded GPCR-Gα_{I} reporter strain, creating four new strains, each with one copy of mTq2 in the X-2, X-3, XI-2, or XII-5 pad. As shown in FIG. 15B, mTq2 expressed from the X-2 and XII-5 pads gave higher fluorescence values than from the X-3 and XI-2 pads (<2-fold difference). Thus, mTq2 was expressed at slightly higher levels from the X-2 and XII-5 pads.

### iv. Testing the Four-Padded GPCR Reporter Strains

Having characterized the individual landing pads in the GPCR-Gα_{I} reporter strain, all 10 four-padded GPCR reporter strains were tested using the proton-sensing receptor GPR68. To do this GPR68 was installed in each four-padded GPCR reporter strain, generating 40 new strains, each with one copy of GPR68 in the X-2, X-3, XI-2, or XII-5 pad. Agonist treatment was not needed in the testing procedure because GPR68 is constitutively active below pH 7. As shown in FIG. 15C, the same GPR68 signaling pattern was observed for all four pads. Furthermore, pad-dependent differences in GPR68 signaling were the same as for mTq2 pad testing (FIG. 15B), indicating that GPR68 was also expressed at slightly higher levels from the X-2 and XII-5 pads.

### v. Potentiating Gα Coupling by Incrementally Increasing GPR68 Gene Copy Number

As shown in FIG. 15C, GPR68 signaling was strongest in the four-padded GPCR-Gα_{z} reporter strain. However, GPR68 has also been reported to signal through other Gα subunits, such as Gα_{Q}. A likely explanation for the observed GPR68-Gα coupling pattern is that GPR68-Gα interactions depend on receptor expression levels. To demonstrate the utility of the four-padded GPCR reporter strains in an application, an experiment was performed to test this possibility.

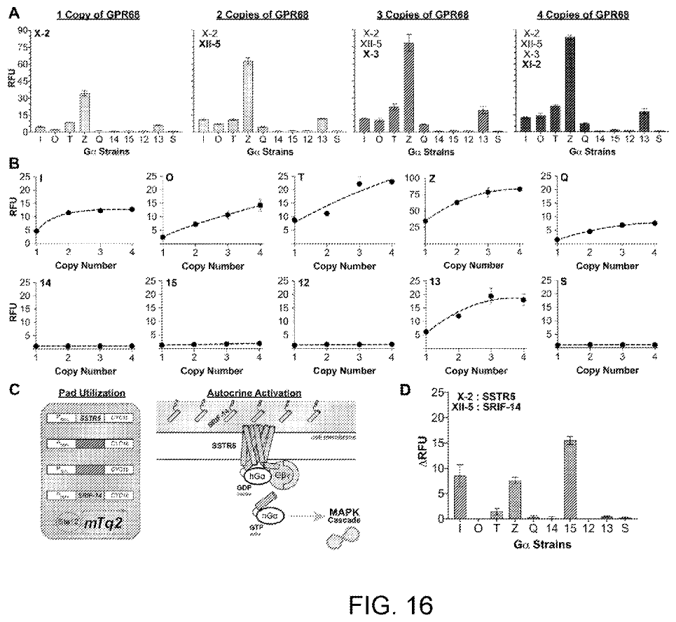

As shown in FIG. 16A, one, two, three, and four copies of the GPR68 gene were installed into the X-2, XII-5, X-3, and XI-2 landing pads, generating 30 additional new strains. Incrementally increasing GPR68 copy number led to increased signaling through the 6 chimeric Gα subunits Gα_{I}, Gα_{O}, Gα_{T}, Gα_{Z}, Gα_{Q}, and Gα₁₃. For example, a 2.4-fold change in signaling was observed between GPCR-Gα_{Z} reporter strains with one and four pads (FIG. 16B). This result was typical of the other GPCR coupling combinations that signaled. Additionally, the emergence of new GPCR coupling combinations with increasing GPR68 copy number were observed. For example, two copies of GPR68, in the X-2 and XII-5 pads, were sufficient to reproduce previously observed Gα_{Q} coupling (FIG. 16B). Sequential addition of a third and fourth GPR68 copy in the X-3 and XI-2 pads increased Gα_{Q} signaling even further, resulting in an overall 5.2-fold change in efficacy.

### vi. Autocrine Activation of SSTR5 With its Peptide Agonist SRIF-14

In a second application of the padded GPCR reporter strains, the somatostatin receptor SSTR5 and its peptide agonist SRIF-14 were co-expressed from the X-2 (SSTR5) and XII-5 (SRIF-14) pads, generating 10 new strains. For this experiment, a version of the SRIF-14 peptide was used that included a N-terminal pre-alpha factor secretion signal (PFSS) and a C-terminal Flol₍₁₄₉₆₋₁₅₃₇₎ sequence. As illustrated in FIG. 16C, this added functionality directs SRIF-14 to be secreted (PFSS) and trapped (Flo1) inside the yeast cell wall where it can readily agonize SSTR5.

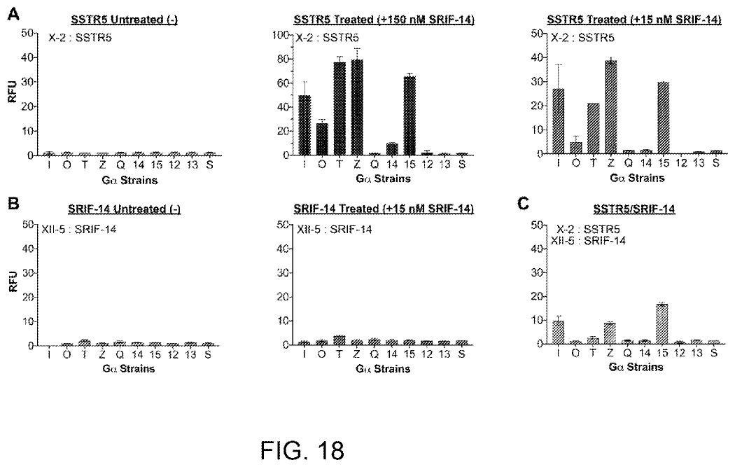

As shown in FIG. 16D, expression of the trapped SRIF-14 peptide agonist from the XII-5 pad was sufficient to elicit autocrine SSTR5 signaling in the Gα_{I}, Gα_{T}, Gα_{Z}, and Gα₁₅ reporter strains. Based on the reported nanomolar EC₅₀ of SRIF-14 for SSTR5, it was speculated that the SRIF-14 peptide was expressed at nanomolar levels from the XII-5 pad. To confirm this, 20 additional GPCR reporter strains were created: 10 with only SSTR5 in the X-2 pad and 10 with only SRIF-14 in the XII-5 pad. Treatment of these 20 control strains with exogenously added SRIF-14 peptide agonist at a concentration of 15 nM resulted in similar SSTR5 signaling levels and the same Gα coupling pattern (FIG. 18).

In principle, this application could be extended to any GPCR-peptide agonist pair, or any interaction between a GPCR and a genetically-encodable ligand, including proteins such as chemokines and nanobodies. Perhaps the greatest benefit of pad-based autocrine secretion is that it circumvents the time and expense of producing and purifying genetically-encodable GPCR ligands in the lab, or purchasing them from commercial sources.

### vii. Engineering and Validating a Four-Padded General-Utility BY4741 Yeast Strain

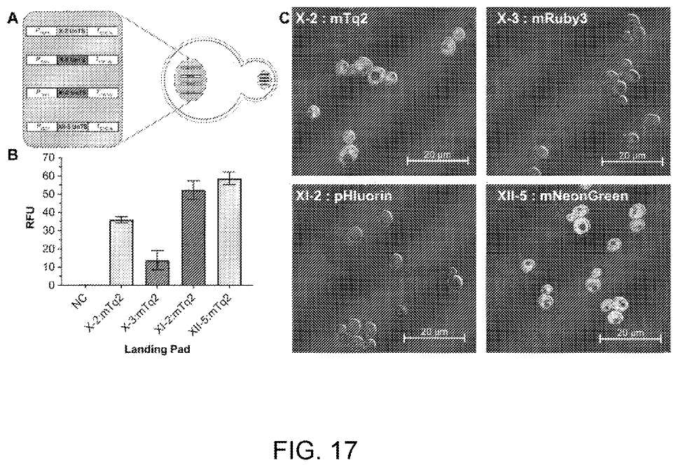

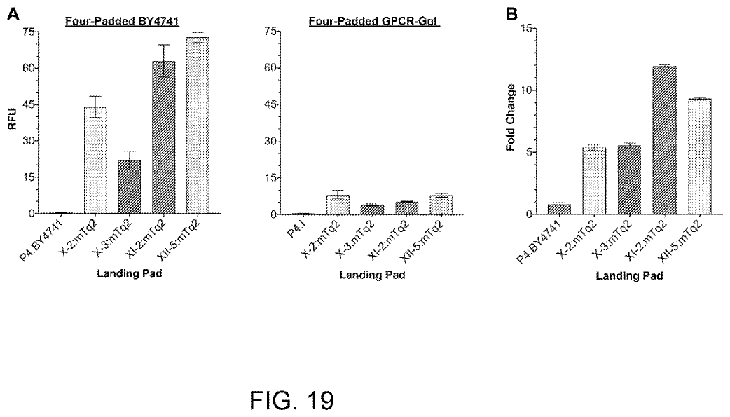

Given the demonstrated utility of the four landing pads in the GPCR reporter strains, a more general-purpose strain in the BY4741 background was generated. To do this, each landing pad was sequentially installed into the base BY4741 strain, creating a new four-padded yeast model (FIG. 17). As with the GPCR reporter strains, mTq2 was used to confirm the functionality of each pad. In all cases, mTq2 fluorescence was brighter in the BY4741 background than the GPCR-Gα_{I} reporter strain background (FIG. 19). This indicated that the genetic manipulations used to engineer the GPCR reporter strains (far1Δ, sst2Δ, ste2Δ) cause a phenotype with reduced mTq2 transcription and/or translation through the constitutive TEF1 promoter. With the exception of the XI-2 pad, the pattern of mTq2 fluorescence intensity was the same in both the BY4741 (FIG. 17B) and GPCR-Gα_{I} reporter strains (FIG. 15B). Notably, both sets of mTq2 pad-testing strains (FIGS. 15B and 17B) demonstrated that mTq2 expression was lowest from the X-3 pad.

Having confirmed the functionality of the four-padded BY4741 strain, its utility was demonstrated using four different fluorescent proteins. To do this we installed mTq2 (J. Goedhart et al., Nat Commun 3, 751 (2012)), mRuby3 (B. T. Bajar et al., Sci Rep 6, 20889 (2016)), pHluorin (G. Miesenbock, et al., Nature 394, 192-195 (1998), and mNeonGreen (N. C. Shaner et al., Nat Methods 10, 407-409 (2013)) in the X-2, X-3, XI-2, and XII-5 pads respectively. This process generated four new strains, each with a single fluorescent protein in one of the four pads. As shown in the confocal microscopy images in FIG. 17C, each fluorescent protein was expressed as robustly as mTq2. This was true even in the case of mRuby3, which was produced from the lower expressing X-3 landing pad. Taken together, these results demonstrated the facile engineerability and reliability of the four-padded BY4741 strain.

All publications and patent applications mentioned in this specification are herein incorporated by reference to the same extent as if each individual publication or patent application was specifically and individually indicated to be incorporated by reference.

1. A plurality of cells, wherein each cell comprises (i) one or more of a target domain gene that specifically binds to a binding partner (ii) one or more of an intracellular chimeric G-protein alpha subunit comprising an endogenous G-protein alpha subunit with a humanized C-terminus; and (iii) one or more of an inducible reporter, wherein the expression of the reporter is dependent on the activation of the target domain encoded by the target domain gene, and wherein the target domain gene comprises a barcode.

2. The plurality of cells of claim 1, wherein the one or more of target domain is a membrane channel, a symporter transporter, an antiporter transporter, an ATPase, an enzyme or a receptor.

3. The plurality of cells of claim 2, wherein the receptor is a G-protein coupled receptor (GPCR).

4. The plurality of cells of claim 1, wherein the one or more of inducible reporter is a transcriptional reporter.

5. The plurality of cells of claim 1, wherein the transcriptional reporter is mTurquoise2.

6. The plurality of cells of claim 1, wherein the cells are yeast cells.

7. The plurality of cells of claim 6, where the yeast cells are Saccharomyces cervisiae.

8. The plurality of cells of claim 6, wherein the yeast cells lack an endogenous GPCR.

9. A method for identifying a compound capable of modulating the activity of a target domain, comprising:
(a) contacting the plurality of cells of claim 1 with a compound;
(b) determining the activity of the target domain by detecting the reporter; wherein detection of the reporter in the cell indicates that the compound interacts with the target domain.

10. The method of claim 9, wherein the target domain is a membrane channel, a symporter transporter, an antiporter transporter, an ATPase, an enzyme or a receptor.

11. The method of claim 10, wherein the receptor is a G-protein coupled receptor (GPCR).

12. The method of claim 10, wherein the compound is a metabolite, lead compound, drug, natural product, or other experimental small molecule, lipid, peptide, or protein.

13. A yeast cell comprising a plurality of landing pads integrated in the yeast cell's genome, wherein each exogenous landing pad is integrated at a safe harbor genome loci in the yeast cell's genome.

14. The yeast cell of claim 13, wherein the yeast cell comprises between 1 to 4 exogenous landing pads.

15. The yeast cell of claim 14, wherein the yeast cell comprises 4 exogenous landing pads.

16. The yeast cell of claim 13, where the yeast cell is Saccharomyces cervisiae.

17. The yeast cell of claim 13, wherein the plurality of exogenous landing pads are integrated at loci X-2, X-3, XI-2, and/or XII-5 of the yeast cell's genome.

18. The yeast cell of claim 13, wherein the plurality of exogenous landing pads comprise a unique targeting sequence.

19. The yeast cell of claim 13, wherein the plurality of exogenous landing pads comprise a unique targeting sequence, a PAM site, and buffer DNA.

20. The yeast cell of claim 13, wherein the plurality of exogenous landing pads are integrated sequentially.

21. The yeast cell of claim 20, wherein the landing pads are integrated sequentially in the following order: X-2, XII-5, X-3, and XI-2.


SEQUENCE LISTING (201 sequences)

SEQ ID NO: 1 - DNA, 260 bp, Artificial Sequence
  FAR1 DP
tgtcttgagagtgtatattatcttatctattcaaaaaatttctatttacttttatatttcttgaccatcctttacacaaagtctatagatccactggaaagcttcgtgggcgtaagaaggcaatctattatagttcgggaatcgaggcccgtatttcgaggcttttgcttttccttttttttttttcgtttctccacgtctatactacgcaatgactgaatatatataatgcgtcgtaaatagcagttaatgtataaata

SEQ ID NO: 2 - DNA, 202 bp, Artificial Sequence
  Ste2 DP
cttcaaagcaatacgataccttttcttttcacctgctctggctataattataattggttacttaaaaatgcaccgttaagaaccatatccaagaatcaaaatcaaaatttacggctttgaaaaagtaatttcgtgaccttcggtataaggttactactagattcaggtgctcatcagatgcaccacattctctataaaaaaa

SEQ ID NO: 3 - DNA, 261 bp, Artificial Sequence
  GPA1_chimera_GNAI3
tttttgagcttgaataagacaaacaaaccaatctacgtgaaacgaacctgcgctaccgatacccaaactatgaagttcgtattgagtgcagtcactgatctaatcatccagcaaaaccttaaagaatgtggtttgtattgaaggaactgtataattaaagtagtgtttagatacgtaaattctgtttccgaagatgcaagaaggagcagcagcaccagaaaaaattactatttttcttctccattagagtctatgatggaa

SEQ ID NO: 4 - DNA, 261 bp, Artificial Sequence
  GPA1_chimera_GNAO
tttttgagcttgaataagacaaacaaaccaatctacgtgaaacgaacctgcgctaccgatacccaaactatgaagttcgtattgagtgcagtcactgatctaatcatccagcaaaaccttaaaggttgtggtttgtattgaaggaactgtataattaaagtagtgtttagatacgtaaattctgtttccgaagatgcaagaaggagcagcagcaccagaaaaaattactatttttcttctccattagagtctatgatggaa

SEQ ID NO: 5 - DNA, 261 bp, Artificial Sequence
  GPA1_chimera_GNAT123_I12
tttttgagcttgaataagacaaacaaaccaatctacgtgaaacgaacctgcgctaccgatacccaaactatgaagttcgtattgagtgcagtcactgatctaatcatccagcaaaaccttaaagattgcggtttgttttgaaggaactgtataattaaagtagtgtttagatacgtaaattctgtttccgaagatgcaagaaggagcagcagcaccagaaaaaattactatttttcttctccattagagtctatgatggaa

SEQ ID NO: 6 - DNA, 261 bp, Artificial Sequence
  GPA1_chimera_GNAZ
tttttgagcttgaataagacaaacaaaccaatctacgtgaaacgaacctgcgctaccgatacccaaactatgaagttcgtattgagtgcagtcactgatctaatcatccagcaaaaccttaaatatatcggtttgtgctgaaggaactgtataattaaagtagtgtttagatacgtaaattctgtttccgaagatgcaagaaggagcagcagcaccagaaaaaattactatttttcttctccattagagtctatgatggaa

SEQ ID NO: 7 - DNA, 261 bp, Artificial Sequence
  GPA1_chimera_GNAQ_11
tttttgagcttgaataagacaaacaaaccaatctacgtgaaacgaacctgcgctaccgatacccaaactatgaagttcgtattgagtgcagtcactgatctaatcatccagcaaaaccttaaagaatataatttggtttgaaggaactgtataattaaagtagtgtttagatacgtaaattctgtttccgaagatgcaagaaggagcagcagcaccagaaaaaattactatttttcttctccattagagtctatgatggaa

SEQ ID NO: 8 - DNA, 261 bp, Artificial Sequence
  GPA1_chimera_GNA14
tttttgagcttgaataagacaaacaaaccaatctacgtgaaacgaacctgcgctaccgatacccaaactatgaagttcgtattgagtgcagtcactgatctaatcatccagcaaaaccttaaagaatttaatttggtttgaaggaactgtataattaaagtagtgtttagatacgtaaattctgtttccgaagatgcaagaaggagcagcagcaccagaaaaaattactatttttcttctccattagagtctatgatggaa

SEQ ID NO: 9 - DNA, 261 bp, Artificial Sequence
  GPA1_chimera_GNA15
tttttgagcttgaataagacaaacaaaccaatctacgtgaaacgaacctgcgctaccgatacccaaactatgaagttcgtattgagtgcagtcactgatctaatcatccagcaaaaccttaaagaaattaatttgttgtgaaggaactgtataattaaagtagtgtttagatacgtaaattctgtttccgaagatgcaagaaggagcagcagcaccagaaaaaattactatttttcttctccattagagtctatgatggaa

SEQ ID NO: 10 - DNA, 261 bp, Artificial Sequence
  GPA1_chimera_GNA12
tttttgagcttgaataagacaaacaaaccaatctacgtgaaacgaacctgcgctaccgatacccaaactatgaagttcgtattgagtgcagtcactgatctaatcatccagcaaaaccttaaagatattatgctacaatgaaggaactgtataattaaagtagtgtttagatacgtaaattctgtttccgaagatgcaagaaggagcagcagcaccagaaaaaattactatttttcttctccattagagtctatgatggaa

SEQ ID NO: 11 - DNA, 261 bp, Artificial Sequence
  GPA1_chimera_GNA13
tttttgagcttgaataagacaaacaaaccaatctacgtgaaacgaacctgcgctaccgatacccaaactatgaagttcgtattgagtgcagtcactgatctaatcatccagcaaaaccttaaacaattgatgttgcaatgaaggaactgtataattaaagtagtgtttagatacgtaaattctgtttccgaagatgcaagaaggagcagcagcaccagaaaaaattactatttttcttctccattagagtctatgatggaa

SEQ ID NO: 12 - DNA, 261 bp, Artificial Sequence
  GPA1_chimera_GNAS_L
tttttgagcttgaataagacaaacaaaccaatctacgtgaaacgaacctgcgctaccgatacccaaactatgaagttcgtattgagtgcagtcactgatctaatcatccagcaaaaccttaaacaatatgaattgttgtgaaggaactgtataattaaagtagtgtttagatacgtaaattctgtttccgaagatgcaagaaggagcagcagcaccagaaaaaattactatttttcttctccattagagtctatgatggaa

SEQ ID NO: 13 - DNA, 1038 bp, Artificial Sequence
  CHRM1 deltail3
atgaatacaagcgctcctccagcggtttccccaaatattacggtgctggcacctgggaaaggaccatggcaagttgcttttatcggtatcaccaccggcttgctctccttggcaacagtcacgggcaatttgctcgtactgatctcatttaaggtgaacacagagctgaaaacagtgaacaactacttcttgttgagtttggcctgtgcggaccttattatcggaacgttttcaatgaatctgtatacaacctatctcctgatgggccactgggcgctgggaacccttgcttgcgacctttggttggcccttgattacgtggcgagcaacgcaagcgtcatgaatctcctgttgatatcatttgacaggtatttttctgttacccggcccctgagctacagagctaaacgaactccacgacgcgctgcactgatgattggtctggcctggctggtcagttttgtcctgtgggcccctgccatactgttttggcagtacctggtgggagagcgcaccgtgcttgcaggtcagtgctatatacagtttctgtctcagcccataatcacattcggcaccgcgatggcggcattttacttgcctgtgactgttatgtgtactctgtattggcgaatttaccgcgaaaccgagaatagagccagggaacttgctgcactgcagggttcagagacaaaggaacagctggcgaagcggaaaaccttcagcctcgtgaaggaaaagaaagccgcacgaaccctgtccgctatcttgctggctttcatactgacctggacgccttataacattatggtgctggtctccaccttttgtaaagattgcgtcccagagacactttgggagctggggtactggctgtgctatgtgaactccacaattaacccaatgtgttatgccttgtgtaataaagcgttcagagatacattccggcttctccttctgtgccggtgggataagaggcgctggcgcaaaatccccaaacggcccgggtcagttcacagaacacctagcagacaatgttaa

SEQ ID NO: 14 - DNA, 1185 bp, Artificial Sequence
  CHRM3 deltail3
atgacgctgcataataattcaaccacaagccctctgttccctaacatcagtagtagctggatacactctcctagcgacgccggacttcctcccggaaccgtgacccactttgggtcatataacgtctctcgggccgcaggcaatttttcttccccagatgggacaaccgacgatccgctcggcggccacactgtatggcaggtggttttcatagcctttctgaccggcattctggctctggtcacaatcatcggcaatattctggtaattgtgtccttcaaggtaaacaaacaactgaagactgtcaataactactttctgcttagtttggcatgcgccgatctgattatcggagtgataagtatgaatctcttcactacttacatcattatgaacagatgggctctgggtaacttggcttgcgacctgtggctcgcaattgactatgtcgcaagcaacgccagtgtgatgaaccttctggttattagcttcgaccgctattttagcattacacgccctctgacctacagagcgaaacgcactacaaagcgggcaggagttatgatcggcctggcctgggtcatttccttcgtgctctgggcaccagccatcttgttctggcagtatttcgtcggtaagaggacagtcccaccaggcgagtgcttcatccaatttttgtccgagccaaccataacattcggcacagccatcgcggcattttacatgccagtgactatcatgaccattctgtactggagaatctacaaagaaaccgaaaagcggacaaaggagttggcagggcttcaggcatcaggcacagagacccggtcacagataaccaagcgcaagcgaatgtcactcgtgaaagaaaaaaaagcagcacagacattgagcgccatcttgctcgcttttatcataacatggacaccctacaatatcatggtcctcgtgaacactttctgcgactcctgtattcctaagactttctggaacctcggctactggctgtgctatatcaacagtacagttaaccctgtgtgctacgctctctgtaacaagacctttaggaccacgttcaagatgctcctcctgtgccagtgtgacaaaaagaaacgacgaaagcagcaatatcagcagcgacagagtgtgatctttcataaacgggcgccagaacaagcgctctaa

SEQ ID NO: 15 - DNA, 1068 bp, Artificial Sequence
  CHRM5 deltail3
atggagggcgactcatatcacaatgccacgactgtgaacggcacacccgtcaaccaccagccacttgaaaggcacaggctttgggaggtaataaccatagccgcagtgactgcagtagttagtctgattaccattgttggcaatgtattggtcatgatttcttttaaggttaacagccagctgaagaccgtgaataattattatctcctttccctggcatgcgctgacttgatcatcgggatctttagcatgaacctgtatactacgtacatcctgatggggcgctgggcactgggttcattggcatgtgacttgtggcttgcgctggattatgttgcatctaacgccagcgtaatgaatcttctcgtgatatcctttgacagatacttctctattacacggcccctgacgtacagagccaaaaggacccccaaacgagcaggcatcatgattggactggcttggctcatatccttcatactctgggcccctgcgatcctctgttggcagtatctcgtggggaaaaggactgtgcccctggacgaatgccagatccagttcctgagtgagcccacaataacttttgggaccgccatagctgctttctatattccggtatccgttatgaccatcctttactgcaggatctacagagagacagagaaaaggaccaaagatttggctgatcttcaggggtctgattctgtgtctactaaaggactcaacccgaatcccagccatcagatgaccaaacgcaagcgggtagtgctggttaaagaaaggaaagctgcacagactctgagcgcgattctgctggcctttatcattacttggaccccctataacatcatggtgctcgtatctactttctgtgacaagtgcgtccccgtgaccctgtggcacctgggctactggctgtgctacgttaattcaacagtgaatcccatctgttatgccctttgtaaccggacctttcgcaaaacctttaagatgctgcttttgtgtaggtggaagaagaaaaaagtcgaagaaaagctttactggcagggcaatagcaaattgccataa

SEQ ID NO: 16 - DNA, 813 bp, Artificial Sequence
  X2-LP-CYC1b
tccatttctttttcctcgggcagagaaactcgcaggcaacttgctctcgaagtggtcacgcacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaattgcgtaagtggcccctagcggggcagtggtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttggcataatcggccctcacagagggatcccgttacccatctatgctgaagatttatcatact

SEQ ID NO: 17 - DNA, 848 bp, Artificial Sequence
  fig1-mTq2
gcttgtctttggtagaagaaattatagtaaacaaacaaacaaacaaacaaaaaaaaaaaaaaaaaaatgtctaaaggtgaagaattgtttactggtgttgttccaattttggttgaattggatggtgatgttaatggtcataaattttctgtttctggtgaaggtgaaggtgatgctacttatggtaaattgactttgaaatttatttgtactactggtaaattgccagttccatggccaactttggttactactttgtcttggggtgttcaatgttttgctagatatccagatcatatgaaacaacatgatttttttaaatctgctatgccagaaggttatgttcaagaaagaactattttttttaaagatgatggtaattataaaactagagctgaagttaaatttgaaggtgatactttggttaatagaattgaattgaaaggtattgattttaaagaagatggtaatattttgggtcataaattggaatataattatttttctgataatgtttatattactgctgataaacaaaaaaatggtattaaagctaattttaaaattagacataatattgaagatggtggtgttcaattggctgatcattatcaacaaaatactccaattggtgatggtccagttttgttgccagataatcattatttgtctactcaatctgctttgtctaaagatccaaatgaaaaaagagatcatatggttttgttggaatttgttactgctgctggtattactttgggtatggatgaattgtataaataattttatcctcaaataaacatataagttttgagcggatatttcagaatgtcaatttttaaaagtaa

SEQ ID NO: 18 - DNA, 848 bp, Artificial Sequence
  fus1-mTq2
tacgacatcctttatcttttttcctttaagagcaggatataagccatcaagtttctgaaaatcaaaatgtctaaaggtgaagaattgtttactggtgttgttccaattttggttgaattggatggtgatgttaatggtcataaattttctgtttctggtgaaggtgaaggtgatgctacttatggtaaattgactttgaaatttatttgtactactggtaaattgccagttccatggccaactttggttactactttgtcttggggtgttcaatgttttgctagatatccagatcatatgaaacaacatgatttttttaaatctgctatgccagaaggttatgttcaagaaagaactattttttttaaagatgatggtaattataaaactagagctgaagttaaatttgaaggtgatactttggttaatagaattgaattgaaaggtattgattttaaagaagatggtaatattttgggtcataaattggaatataattatttttctgataatgtttatattactgctgataaacaaaaaaatggtattaaagctaattttaaaattagacataatattgaagatggtggtgttcaattggctgatcattatcaacaaaatactccaattggtgatggtccagttttgttgccagataatcattatttgtctactcaatctgctttgtctaaagatccaaatgaaaaaagagatcatatggttttgttggaatttgttactgctgctggtattactttgggtatggatgaattgtataaataatgaaaataatattgacgttcgcatttaatctatacctataattctgtacttatatactgttcctt

SEQ ID NO: 19 - DNA, 981 bp, Artificial Sequence
  ADORA1
atgcctcccagtatatccgcgttccaagctgcatacattgggatcgaagtgcttatcgcacttgtgtctgtacccgggaatgtgctggtgatttgggccgtgaaggttaatcaggcactgagagatgctacattttgcttcatcgtctctttggcagtggccgacgtcgccgtgggcgctctcgtcattcccctcgccatcctgattaacatcggacctcagacctactttcacacctgtctgatggtcgcatgcccagtgcttattcttacccagagcagcatcctggcacttctggcaatcgccgttgaccgatacctgagagtaaagatcccacttcggtacaaaatggtggtgaccccccggcgagctgctgtagctatagcaggatgttggatcttgagctttgtagttggactgacaccaatgtttggatggaacaatctctcagctgtggagagagcctgggcagccaacgggtccatgggcgagcccgtcattaagtgtgaatttgagaaagtcatctctatggagtatatggtgtacttcaattttttcgtgtgggtgctcccgcccctcctgcttatggtcctgatctatctggaggtgttttatttgatccggaaacaactgaataagaaagtttccgccagttccggcgatccgcaaaaatactatggtaaagagctgaaaatcgctaagagtctcgctctcatcctcttcttgttcgctcttagctggcttcccctccatattctcaactgcataactctgttctgcccctcttgtcacaaaccatccatcctgacttacattgccatctttctcacccacggaaactctgctatgaaccctatcgtgtacgcttttagaattcagaagtttagggtgacttttttgaagatctggaacgaccacttccggtgtcagcccgccccacccattgacgaggacctgccagaggaacggcctgacgactaa

SEQ ID NO: 20 - DNA, 1239 bp, Artificial Sequence
  ADORA2A
atgcctatcatgggttctagcgtgtatatcactgtagagcttgcaatcgcggtcttggccatcctcgggaacgtcctcgtgtgttgggccgtatggctgaatagcaacctccagaacgtgaccaattactttgtggtgtctcttgccgcggctgacattgccgtaggggtgctcgcgatcccgttcgccattacaatctccaccggcttttgcgctgcatgtcatgggtgtttgtttatagcctgtttcgtcctcgttctgacacagtctagcatcttcagtttgctggccatcgccattgataggtatatcgccatccggatcccacttcgctataacgggcttgtcacagggacacgcgccaaaggaatcattgcaatctgctgggtgctgtcatttgccatcgggctgacaccgatgttgggctggaataattgcggtcagccgaaggagggcaaaaaccattctcagggctgtggggagggtcaggtcgcctgccttttcgaggacgtagtccccatgaactacatggtctattttaattttttcgcatgtgtgcttgtgccgttgctgttgatgctgggggtgtatctcaggatttttttggccgctcgccgccagctcaagcagatggaatctcagcccttgcctggcgagcgcgcccggtccactctccagaaggaagtgcatgctgcaaaaagtctggccattattgtaggccttttcgccttgtgttggctgcccctgcatatcattaactgctttacattcttttgcccagactgctcccatgcacccctgtggcttatgtacctggcaatcgtgttgtcccatacgaactccgtggttaacccattcatatatgcctataggattcgggaatttagacaaacattccgcaaaatcatacgctcccacgttctgaggcaacaggagccttttaaggccgcaggtaccagcgcccgcgtgctggccgcccacggtagcgacggtgaacaagttagtctgaggctgaatggtcatcctccgggcgtgtgggctaacggcagtgcccctcaccctgaaaggaggcctaacggttacgctctcggattggtttctggtggcagcgcgcaggagagccagggcaatacagggctccccgacgtggagctcctgagtcacgagctgaaaggcgtctgccctgagcctcccgggctggatgaccctctggcgcaggatggtgctggggtttcttaa

SEQ ID NO: 21 - DNA, 999 bp, Artificial Sequence
  ADORA2B
atgttgcttgagacccaggatgccctttatgtggccctggaactggtcatcgcggctctcagcgtggctggaaatgtgctggtgtgcgctgccgttggaacagcgaacacccttcagactccaactaattattttctcgttagcctggcagccgccgacgtggctgtaggtctgttcgccattccattcgccatcactatcagcctcgggttttgcacggacttctacggatgccttttcttggcttgcttcgtgctcgttcttactcaaagtagcatatttagcctgctggcagtggccgttgaccgatacctggctatctgcgtgccactgaggtataagagcctggtaaccggaaccagagcacgcggcgttatcgctgtgctttgggttctcgccttcgggatcggcttgactccttttctcgggtggaactcaaaagatagcgccaccaacaactgcaccgagccttgggatggaactaccaatgaaagctgctgcctcgtgaagtgcctgttcgagaacgtggtgcccatgtcctacatggtgtacttcaacttcttcggctgcgttttgccccccttgctgataatgttggtgatttatattaagatttttctcgtggcctgtcgacagctccagaggaccgagctgatggaccacagccgaactacgctgcaacgagaaattcacgccgccaagagcctggccatgattgtgggtatctttgcactgtgttggttgccagtgcacgcggtgaattgtgttactctgttccaaccagctcaggggaaaaacaaacccaaatgggcgatgaatatggcaatcctcctgtctcatgccaatagcgttgtcaatccaatcgtgtatgcctatagaaaccgcgacttcagatatacctttcacaaaatcatatccaggtatcttctctgtcaggcggatgtgaagtccggcaatggccaggctggcgtacagcctgccttgggagtggggctgtaa

SEQ ID NO: 22 - DNA, 1398 bp, Artificial Sequence
  ADRA2A
atgttccggcaggagcagccactcgccgagggttcctttgcccccatgggaagtcttcagcctgacgccggcaacgctagctggaatggcaccgaagcgcccggtggaggggctcgggctaccccatattctctccaggtcacactgaccctggtttgtctggccggtctcctgatgctgctgacggtctttggcaatgttttggtgataattgcggtgttcacctccagagcactcaaggctccccagaacctgttcttggtcagcctggcgtctgccgatattctggtcgccaccctcgtaattccgttttctcttgccaatgaggttatgggttactggtattttggtaaggcatggtgcgagatataccttgctctcgacgttctcttctgcaccagcagtattgtgcatctgtgcgcaattagtctggacagatattggtccatcacgcaggcaatagaatataacttgaaaaggactcctcggcgcattaaggctatcatcatcactgtctgggtcattagcgctgtgataagcttccctccacttatttccattgaaaaaaaaggaggaggcggagggccgcagcctgcggagccccgctgtgagatcaacgatcagaaatggtatgtcatctcctcctgcattgggtccttcttcgccccatgtctgatcatgatcctcgtatatgttaggatttatcagatagctaagcgaaggacccgggtccccccatccaggagaggtccagacgcagtggccgctccccccggaggcacagaaaggagacctaacggtctgggaccagagcgctccgcaggacctggaggtgcagaagccgagcccttgccgacacaactgaacggagctcccggcgaaccagccccagcaggtcctcgcgatactgacgcactggacctcgaagagtcctctagcagcgatcatgcagagcgccctccagggcctaggagacctgaaagaggacccagaggtaaaggcaaggcccgggcaagtcaagtgaaaccgggagactctctgcctcggaggggtccaggtgctactggtatagggactcctgccgccggtcccggagaagagcgggtcggcgccgctaaggcaagtcgctggcggggcagacaaaaccgggaaaaaaggtttacattcgtgctcgcggtagtgattggcgtcttcgtcgtgtgctggtttccattcttttttacctataccctgacagccgtcggctgttctgttccacggacactgttcaagttctttttctggtttggctactgtaactcaagcctgaacccggtaatctatacaatatttaaccacgattttcggagggcttttaaaaagatcctgtgcagaggcgataggaaacgaatcgtttaa

SEQ ID NO: 23 - DNA, 1353 bp, Artificial Sequence
  ADRA2B
atggatcatcaggatccctattctgtccaggccactgcggccatcgctgccgctatcacttttctcattctctttaccatcttcgggaacgcgcttgtaattctggcggtgctcaccagcaggtcactcagggcaccacaaaacctttttctggtctccctcgcggcagctgacattctcgtcgcaaccctcatcattcccttttcactcgctaatgagttgttgggatattggtattttcgccgaacctggtgcgaggtgtatctcgccctggacgtactgttttgcacctcttcaattgtccatctttgtgcaatcagcctggacagatattgggccgtgtcaagggctctggagtataatagcaagcggacacctaggaggatcaagtgcataatccttacagtctggctcatagcggcagtaataagcctgcccccccttatatacaaaggagatcagggccctcaacccagagggcggccacaatgcaagctgaatcaagaggcttggtacattttggcgagcagcatcggaagcttctttgccccatgcctgatcatgattttggtatacctcaggatttacctgatcgctaagcggtctaatagacgcgggcctcgggctaagggaggaccaggccagggggaatccaagcagcctaggcctgatcacggcggcgcacttgccagtgccaagctgcccgcactggcctccgtcgcgtcagcaagagaagtcaatggccacagtaaatctactggtgaaaaggaagagggcgagacccctgaagataccggaacccgcgcactgcctccaagttgggctgcgctgccaaactctgggcagggacagaaggagggggtgtgcggcgcaagtcccgaggacgaggccgaggaggaggaagaagaagaggaggaagaggaggaatgcgagcctcaagcagtgcctgtaagtccagcctctgcatgctcacctcctcttcagcagccacagggctccagagtcctcgctaccctcagagggcaagtgctgctcggaaggggtgtcggtgccattggagggcaatggtggcgccggcgggcacagctgaccagagaaaagcgcttcacctttgtgctcgccgtcgtgatcggcgtattcgttctctgctggttcccatttttcttctcctactccttgggcgcgatttgtcctaagcattgcaaagtaccacatgggctctttcaattttttttctggattggttattgcaatagctcactgaatcctgtgatttacaccattttcaatcaggatttccggcgggcctttagacgcattctgtgcaggccgtggacccagactgcatggtaa

SEQ ID NO: 24 - DNA, 1116 bp, Artificial Sequence
  AVPR2
atgctcatggcctctaccacaagcgctgtgcccggacatccatctcttccaagtcttccctccaacagctcacaggagaggcctctggatacccgggacccacttcttgcaagagcggagctcgccttgctctccatcgttttcgttgccgtcgcgttgagcaatggtctggtgctggccgccctggcgcgacgaggccgccgaggccactgggcgcctattcatgtctttatcggccatctctgtcttgcagacctggccgtggcgctgtttcaggtgctccctcaactggcttggaaagccaccgatagattccgggggccagacgcactgtgtcgggccgtgaaatacctgcagatggtgggaatgtatgccagttcatatatgatacttgctatgaccttggacagacacagggctatctgtcgccccatgctggcgtataggcacggttccggagcccattggaacaggccagttctggttgcgtgggctttttccctgcttctgagcttgccccagttgtttattttcgcacagagaaatgtggagggcggatctggcgtgaccgattgctgggcttgcttcgctgagccatggggcagaaggacctacgtgacgtggattgctctcatggtgttcgttgctcccaccctggggatcgccgcatgccaggtcctgatattcagagaaatacacgcctccttggttcctgggccaagcgagcgccctggtggaagaagacggggtcgaaggactggctctccaggcgaaggcgcccacgtatcagctgcggtagctaagaccgtacgcatgacactggtcatcgtcgtcgtttacgttctttgctgggccccgttttttttggtgcagctctgggcggcctgggaccccgaagcacccctggaaggtgctccattcgttctgctcatgcttctcgcttcactgaatagctgtaccaatccttggatttacgcctctttctcctccagtgttagctccgagctgcggagtcttctctgttgcgcccggggcaggacccccccaagcctgggtccacaggacgaatcctgcactaccgcctccagttccctggccaaagacacctcttcttaa

SEQ ID NO: 25 - DNA, 1083 bp, Artificial Sequence
  CNR2
atggaagagtgttgggtgacagagatcgccaacgggtccaaggatggcctggattcaaatccaatgaaggattatatgatcctgtccggcccacagaagaccgcggttgctgtgctgtgtactcttttggggctgctgagcgcccttgaaaatgtggctgtattgtatctgatcctgtcaagccatcagttgcgccgaaagccctcctatttgtttatcggctcactggccggtgctgacttcctggcaagtgtcgtatttgcgtgctcctttgtcaattttcacgtgtttcacggagtcgacagcaaggcagtattcctgctgaaaatcggcagcgtaacaatgacttttacggcttctgtgggctccctcctcctgaccgcaatagaccgatacctgtgtttgcggtacccgccatcatacaaagccctccttacaagaggacgcgctctggtgacactcggcattatgtgggtactctcagctctggttagctatcttccactgatgggatggacgtgttgtcctcgcccttgttcagagctttttcctctgattcccaatgactacctgctctcctggcttctgtttattgccttcttgttttctggaatcatctatacatatggtcacgtgctctggaaggcacaccagcatgttgcctctcttagcggtcaccaggatagacaggtccccgggatggccaggatgaggttggatgtgagactcgcgaagacactcggcctcgtgctggccgtgctgttgatatgctggtttccggtcttggccctgatggcacacagtttggccaccacactgagtgatcaagttaaaaaggccttcgccttttgtagtatgctgtgcctgataaactccatggttaatccggtcatctacgctcttaggtccggcgagattcgaagctcagcgcaccactgtctggcacattggaaaaaatgcgtgaggggactcggctctgaggccaaggaagaggcaccaaggtcatcagtcaccgagactgaggcggacggaaaaattaccccttggccagattctagggacctggacctcagcgattgctaa

SEQ ID NO: 26 - DNA, 993 bp, Artificial Sequence
  FFAR2
atgcttccagactggaaatcttccctgatactgatggcctacataattattttccttacaggtctccctgctaatctgctggccctgagggcttttgtcggcaggatccgccagccccaacccgcccctgtccacatcctcttgctgtccctgaccctggcggatctcctgttgctgcttcttctcccttttaagattattgaagccgcttccaatttcaggtggtacttgcccaaggtggtatgcgcccttacctcctttggcttctacagtagcatctactgctccacttggcttttggcaggaatctccattgagcgctatctcggcgtagcattccccgtgcagtacaagctctcacgccgccctctctatggagtgatagctgcactcgtggcttgggttatgagtttcggccattgcaccatcgtgattatcgtacagtatctgaataccaccgagcaggtgaggagcgggaacgaaataacatgttacgagaattttacggataatcagctcgacgtcgttttgccggttcggctggaactgtgcttggtgctgttcttcatcccaatggcagttaccatcttttgttattggagatttgtgtggatcatgctttcccaacctttggtaggggcacagagaaggcgccgagctgttggattggctgttgttaccctgctgaatttcctcgtgtgctttggcccatataatgtgagccacctggttggctatcatcagcgcaagtccccatggtggcgcagcatagccgtggtgttttccagcctcaatgcaagcctggatcctctcctgttctacttcagcagtagcgtcgtgcgccgggcttttggaagaggactccaggtactgaggaaccagggtagcagcttgctcggcaggcggggaaaggataccgcagaaggcacaaacgaggatcgaggagtcggtcagggcgaggggatgccatcatccgacttcacaactgagtaa

SEQ ID NO: 27 - DNA, 930 bp, Artificial Sequence
  GPR35
atgaacggcacgtataacacatgtggaagtagtgatttgacctggccccctgctatcaagctcggcttttacgcctacctgggtgttcttctggtactgggattgctcctgaactccttggcactctgggtgttttgctgtagaatgcaacagtggacagaaactcgcatttacatgaccaacctggctgtggctgacctgtgccttctgtgtacgctgccctttgtccttcattccctgcgggatacttccgatacccccctgtgccagctgagccaaggtatttatctgactaataggtatatgagcatcagccttgttactgcaattgcagttgacagatatgtagcggtgagacatcctctgcgcgctcgggggcttagatcaccgaggcaggcagctgccgtgtgcgctgtgctgtgggttcttgtcattggcagcctggtcgcgaggtggctcttgggtatccaggaaggtggtttttgtttccgatcaacacggcacaatttcaactcaatggcttttcccctgctcggcttttatctgcctcttgccgtggtggtgttctgttccctgaaggtcgttaccgccctcgcccagagaccccccaccgacgtgggccaggctgaggcgacgagaaaggctgccaggatggtgtgggcaaaccttctggtttttgtggtctgtttcctgccgcttcacgttggacttactgtgagactcgcagtgggctggaacgcttgcgctttgctggagacaatccgccgagcgctgtatattacatcaaagctgtcagacgcaaactgctgcctcgacgctatctgctactactacatggcaaaggaatttcaagaggcttctgcacttgcagtagcccccagtgccaaagctcataagtcacaggattctctgtgtgtgacactggcctaa

SEQ ID NO: 28 - DNA, 1089 bp, Artificial Sequence
  GPR4
atgggcaaccatacctgggaagggtgccacgtggattcacgggtggaccatcttttcccaccctccctgtacatttttgtcatcggggtgggcttgcccacaaactgccttgcactgtgggcagcctatcgacaggtgcagcaaagaaacgaactgggcgtgtaccttatgaatctgtctattgccgatctcctctacatctgcacactgcctctttgggtcgactacttccttcaccatgacaattggatccacggtccagggtcttgtaagctttttggtttcattttctacacaaatatttatattagtatcgcttttctctgctgtatcagcgtggatcggtacctggccgtggcgcacccactccggttcgctcgactcaggcgagttaagactgcagtggcggtatcaagcgtggtttgggccacagagttgggcgcaaattccgcacccctgtttcatgacgagttgttccgggaccgatacaatcacacattttgtttcgaaaagttccccatggaagggtgggtagcctggatgaacctctacagggtcttcgtgggcttcctgttcccatgggctcttatgttgctctcttaccggggcatcctgagggctgtgagaggttccgttagtacagaaagacaggaaaaagccaagattaaacggcttgcgctgtccctgatcgccatcgtccttgtctgctttgctccctaccacgtcctgctgctgagcaggtccgccatctatctgggccgcccttgggattgcggattcgaggagcgggtgttctcagcctatcattcctcactggcatttaccagcttgaattgcgttgccgaccctattttgtactgcttggtgaacgagggcgcaaggagtgatgttgcaaaggctcttcacaacctgctcagattcctggcctctgataaaccacaggagatggccaatgcttctctgacattggagacacctttgacaagtaagcggaattccaccgcgaaagctatgacagggtcatgggccgccacccctccctcacagggcgaccaggtgcaattgaagatgctgcccccagctcagtaa

SEQ ID NO: 29 - DNA, 1014 bp, Artificial Sequence
  GPR65
atgaacagtacttgcatcgaagagcaacacgacctcgaccattacctgttccctattgtgtatatttttgtcatcatcgtgtcaattccagcaaacataggctctctctgcgtcagtttccttcaggccaaaaaggagagcgagcttggcatctacctgtttagcctcagtcttagcgacctcctgtacgcacttacactccccctctggatagactatacttggaataaagacaactggaccttcagtccagcactttgtaaaggctcagctttcctgatgtacatgaacttttacagttccaccgcttttcttacatgcattgcagtcgaccgctaccttgccgtagtctacccactgaaattcttttttctgagaaccaggagattcgccttgatggtgagcttgagcatttggattctcgaaaccatattcaacgccgttatgctctgggaagatgaaacggtagttgaatattgcgatgctgagaagtccaactttacactgtgctacgataagtatcccctggagaagtggcagattaacctcaacctttttcgcacatgcacaggctacgctatcccactggtgactattctgatttgtaaccggaaggtctaccaggcggtccggcacaacaaagccactgagaacaaggagaaaaaacggattataaagctgctggtgtcaataactgtgacgttcgtgttgtgcttcacccccttccacgtgatgctgctcataagatgcatccttgagcatgccgtgaacttcgaagaccatagcaattccgggaagaggacttatacaatgtacaggatcactgttgcccttacatctctcaactgcgtggctgatcctattctgtattgcttcgtcaccgaaactggtcgctacgatatgtggaatatactgaagttctgtaccggacgctgcaacacaagccagagacaacggaagagaatactgtctgtttctacaaaggacaccatggagctggaagtcctggagtaa

SEQ ID NO: 30 - DNA, 1098 bp, Artificial Sequence
  GPR68
atggggaacatcactgccgacaatagctccatgagctgtaccatcgaccacactatccaccagaccctcgctcccgtggtttacgttacggtacttgtcgtaggcttccctgccaactgcctcagtctctactttgggtacctgcagataaaagctcgcaacgagttgggagtgtacttgtgcaacctgacagtggcagacctgttctacatatgctcccttcccttttggctgcagtacgtcctccagcatgataattggtcccatggtgacctgagctgccaagtgtgcgggattcttctttatgagaatatctacatttccgtgggattcctttgctgcatcagcgtggaccgctacctggccgtagcccatcctttccgattccaccagttccggacactcaaggctgcagtgggggttagcgtggtgatctgggctaaagaactcctgacctcaatatacttcttgatgcacgaggaagtgatcgaggacgagaatcagcacagggtctgcttcgagcactaccctattcaagcttggcagcgagctatcaattactacagatttctcgtggggttcctgtttcctatctgtctgctgctggcctcctaccagggaatcctgcgagccgttcgccgcagccatggtactcagaaatctcgcaaagaccaaattcagagacttgtgctcagcactgtggtcattttcctcgcgtgcttcctgccatatcatgtgttgctgctggtcagaagcgtgtgggaggcatcttgtgacttcgccaagggagtctttaacgcctatcatttcagtctccttctgacatctttcaattgtgtcgccgatcctgtgctctattgcttcgtttccgaaacaactcatagggatcttgcacgcctccgcggagcttgcctggccttccttacatgttcaaggaccggcagggcacgcgaagcatatccgctgggtgccccagaggcttctggtaagtcaggcgcgcaaggagaggagccggaactgcttaccaagctccaccccgcttttcaaacccccaactcccccgggtccggcggctttccaaccgggagacttgcataa

SEQ ID NO: 31 - DNA, 1092 bp, Artificial Sequence
  HCAR2
atgaaccggcaccacctgcaggatcacttcctggaaatagacaaaaagaactgctgcgtttttagagacgactttatcgtcaaagtactgcccccagtgctcggcctggagttcatcttcggacttctggggaatggcctcgccctctggattttttgcttccaccttaagtcctggaagagtagccgaatcttcctgttcaatctcgccgtagcggattttctgctcatcatttgcctgcctttcctgatggacaattatgtgcgccggtgggattggaaattcggtgatattccttgtagattgatgctcttcatgctggctatgaaccggcaaggaagcataatctttcttaccgttgtggccgtggacagatacttccgagtggtgcatccacatcatgccctcaataagatttccaatcgaacggccgctataattagctgtcttctgtggggaatcaccatcggcctcaccgtccacctcctcaagaagaagatgccgatccagaacggtggggccaacctgtgtagttcctttagtatatgccatactttccagtggcatgaggctatgttcctgctggagttcttcctgccacttggcatcatcttgttctgctccgctcggataatatggagcctgagacagagacagatggatcggcatgcgaaaataaagcgggccattacgttcatcatggtcgtggctattgtgtttgtgatttgtttcctcccctctgtggtggtgagaataagaattttttggcttctccacacgagcggcacccaaaattgcgaggtttacaggagcgtggatcttgcctttttcatcactctgagcttcacatacatgaattcaatgctcgatccagtggtttactacttcagcagtccttcattcccgaactttttctctacgctcatcaatcggtgcctgcagcgcaaaatgactggcgagcccgacaacaacagaagcacctccgtggaactcaccggcgaccctaacaagacccggggtgcccccgaggctctgatggcgaactctggtgagccctggtcccctagctatctgggcccaacgagcccctaa

SEQ ID NO: 32 - DNA, 1164 bp, Artificial Sequence
  HCAR3
atgaatcgccaccacctgcaggatcactttctcgaaatcgacaagaagaactgttgcgtcttccgggacgattttatagccaaagtgctcccacccgtgctgggcctggagttcatctttggcctcttgggcaacgggttggccctttggatcttctgcttccatctcaagagctggaaatcttcccggattttcctcttcaatttggccgtagcagatttcctgctcatcatctgtcttcctttcgtcatggactactatgtgcgcagaagcgattggaagtttggtgacattccctgccgcttggttttgtttatgtttgctatgaaccgacagggtagcatcatctttctgaccgtcgttgccgtcgaccgatattttcgggtggtgcatcctcaccatgccttgaacaaaatatccaactggacagccgctataatttcttgcttgctgtggggcattactgtggggctgactgtgcaccttctgaagaagaaattgctgatccagaatgggaccgcgaacgtatgcatttctttctctatatgtcatacatttcgatggcatgaggccatgttcctgctggagttttttcttccactcgggattatccttttctgtagtgctaggatcatctggagtttgcggcaacgccaaatggatcggcatgctaaaattaagcgagccataacgttcattatggtggttgccatagtgttcgtgatctgttttctgcctagcgtcgtcgtgaggatccacatcttttggctgctgcacacctctgggacccagaactgcgaagtgtaccgatcagtagatctcgcgttttttatcacattgtcctttacttacatgaactccatgctcgaccctgtggtttactacttcagttctccctcttttccaaacttcttttccaccctgattaatcggtgccttcaaaggaagattacaggagagcctgataataacaggagcacttcagtcgagctgaccggagatcccaacaagactcggggagcccctgaggcacttatcgccaattctggagaaccctggagccctagctacctggggccaacatctaataaccattctaagaaggggcactgccaccaggagccagccagcttggagaaacagttgggctgttgtatcgaataa

SEQ ID NO: 33 - DNA, 1167 bp, Artificial Sequence
  HTR4
atggataagctggatgctaacgttagcagtgaagaaggatttggcagtgttgagaaggtggtgctgctcaccttcctgtccactgtgatcctgatggcaatcttgggtaacctgctcgtcatggtagccgtttgttgggataggcagctgcggaaaatcaagactaactatttcatagtgtctctcgcctttgcagacctcctggttagtgtgctggtgatgccattcggagccatcgaactggtgcaggacatttggatctatggggaagtgttctgcctggtgagaacttcattggatgttctgctgactactgccagcatttttcacctttgctgtattagcctggacagatattacgcaatctgctgccagccactggtgtacaggaataaaatgacacctttgaggatcgccctgatgctcgggggttgctgggttatccccacctttataagtttcttgccgattatgcagggttggaataatatcggaataatagacctcattgagaagcgcaagttcaatcaaaattccaactctacctactgtgtattcatggtaaacaaaccgtacgctataacttgcagcgtggtcgcattctacattccctttctccttatggtgctggcctattatagaatctacgtgacagcaaaggaacacgcacaccagatacagatgcttcagagagccggagccagttctgaaagtaggcctcagtcagcagatcagcattcaacccataggatgcgcactgagaccaaagcagcaaaaactctttgcattattatgggatgcttctgcttgtgctgggcaccattttttgtaaccaacatcgtcgatcctttcattgactacacagtgcccggacaggtgtggaccgccttcctctggttgggctatatcaatagcggcctgaatccttttctctatgccttccttaataaatccttcaggcgcgcatttcttattatattgtgttgcgatgatgaacggtataggagaccttctatcctgggacagaccgtcccttgctcaactacaactatcaacggtagcacacatgttctgcgcgacgccgtcgagtgcggcggtcaatgggagtcccagtgtcatcctcccgctacctctccactcgtcgcggctcaaccttcagatacttaa

SEQ ID NO: 34 - DNA, 1095 bp, Artificial Sequence
  LPAR1
atggccgctattagcaccagtatcccagttatctctcagccccagtttacagcgatgaatgagcctcagtgcttttacaacgaaagcatcgcctttttctataacagatccgggaagcatctggccaccgaatggaatacggtgtctaaattggtcatgggccttggtattaccgtttgcatctttatcatgcttgcaaacttgctggtgatggtggctatctacgtgaaccgcagattccattttccaatttactacttgatggcaaatcttgccgcagctgacttcttcgcggggttggcatatttctacttgatgtttaataccgggcccaacacccgcagacttactgtctcaacttggctgttgcgccagggactcatcgacacttccctgactgcctctgtggcaaacctgttggccatcgcaattgagagacacataacggtgttccgaatgcaacttcatacacggatgtcaaaccggagggtggtggtggtgatcgtggtgatctggaccatggctatcgttatgggcgccattcctagcgtgggttggaattgcatttgcgacatcgagaactgttccaatatggcacctctgtattctgacagttatctggttttctgggccatctttaatctcgtgacatttgtcgtgatggtggtactgtatgctcacatcttcggatacgtgagacaacgcacaatgcggatgtctcgacacagcagcggacccagacggaatcgggacacaatgatgtccctcctcaaaaccgtcgtaatcgtgttgggcgcatttatcatttgttggacccccgggctcgtactgcttctgctcgacgtgtgttgtccccagtgtgacgttcttgcctatgagaagttctttcttttgttggccgaattcaactccgcaatgaatcctattatttactcttatcgagataaagagatgtccgcaacttttcggcagatcctctgctgccagaggagcgagaatcctacaggccccactgaaggttcagatcggtcagcttcatccctcaaccacacaatcctggccggtgtacactccaacgatcattccgtggtctaa

SEQ ID NO: 35 - DNA, 1113 bp, Artificial Sequence
  LPAR4
atgggtgaccggcgattcattgactttcaattccaggattccaatagctccctgcggcctcgattgggcaatgccacagctaacaacacttgtatagtcgatgattcctttaagtacaatctgaacggcgccgtttattccgtggttttcatcctcgggcttattacaaattccgtgtccttgttcgtgttctgctttaggatgaaaatgcggtccgaaaccgccatttttattaccaaccttgccgtctcagatttgctgttcgtttgtaccttgcccttcaagatcttctacaatttcaaccggcactggccatttggggataccctgtgcaaaatcagtggaacggcctttctgaccaatatctacgggtccatgctgttcctgacatgtatcagcgtggacaggttcctggccatagtctatccatttcggtcacgaacgatccgcaccagaaggaactccgctatagtgtgtgcgggagtgtggattctcgtgctctctgggggtatttctgcttctctgtttagcaccacgaatgtgaataacgcaaccactacatgtttcgagggtttttcaaagcgagtctggaagacgtacctgtcaaagattacaatttttatagaagtggtagggtttatcatcccactgattctgaatgtgagttgttcatccgtggtcttgaggactttgcggaaaccagcgacgctctcccagatcggcacaaataagaagaaagtgctcaagatgatcacagtccatatggcggtgttcgttgtgtgcttcgtgccgtataattctgtgcttttcctgtatgcactggtgcggagtcaggccatcaccaactgcttcctggaaaggttcgctaagattatgtatcctattacattgtgcctcgctaccctgaactgctgcttcgaccctttcatctattatttcacactggagagttttcagaagagcttctacattaacgcgcacatccgaatggaatcactgtttaagacagaaaccccactgacaactaaaccctctctgcctgcgattcaggaagaagtgtccgaccagacaacaaacaatggcggtgaactgatgctggaaagtaccttttaa

SEQ ID NO: 36 - DNA, 966 bp, Artificial Sequence
  MRGPRD
atgaaccagacgctgaactccagcggcactgttgagtctgctctgaactactcacgggggagtactgtgcataccgcctacctggttcttagctcacttgccatgtttacatgcttgtgcggcatggccgggaacagtatggtcatctggttgctgggttttagaatgcatcgaaatcctttctgcatctatatactgaacctggccgccgccgatctgctgttcctttttagcatggctagcaccctgtcacttgaaactcaacccctcgtaaatactaccgataaggtgcacgagttgatgaagaggctcatgtattttgcctacacagtggggctgagcctgttgactgctatctctacacagcggtgtcttagcgtcttgttccctatctggtttaaatgtcaccggccaagacaccttagtgcctgggtctgcgggctgctgtggaccctgtgcctcctcatgaacggtctgacaagcagtttttgttctaaatttctgaagtttaatgaagaccgctgcttccgggtagacatggttcaggcagctctgataatgggagtgcttacaccagttatgaccctgtcctctctcacgctcttcgtatgggtgcgcagaagtagccaacaatggcggagacagccgaccagacttttcgtcgtggtgcttgcaagtgtcctggtatttctgatatgttctttgccgctcagtatatattggtttgtcctgtactggctttcattgccccccgaaatgcaggtattgtgtttctctctttcccggttgagctcttccgtctcatcaagtgctaatcccgtgatttactttctggtagggtctaggaggtcccaccggctgccaaccagatctctgggcaccgtgttgcagcaggctcttagggaggagccagaacttgagggaggggaaacccccacagtcggaactaacgaaatgggagcataa

SEQ ID NO: 37 - DNA, 1053 bp, Artificial Sequence
  MTNR1A
atgcagggcaacggttcagcactcccgaatgcaagccagccagtgctgcggggagacggcgcccgacctagttggttggccagcgccctggcctgcgtccttatcttcaccatcgtggtcgacatcctgggcaatctgctggtcatcctgtcagtttataggaacaaaaagctgcggaatgccggcaacatatttgtggtctcactcgctgtagcggatcttgttgtcgccatctacccatatccactcgtgctcatgagtatttttaataacggttggaacctgggttacttgcattgtcaagtgagcggatttttgatgggcctgtctgtcatcggaagcatattcaatatcacaggcattgccatcaacaggtattgctacatatgtcacagtctgaaatacgacaagttgtatagctcaaagaatagcctgtgttacgtcctgctgatatggctgctgacccttgctgccgtcctgccgaatctgagggcaggcaccctgcaatacgatcccagaatttattcatgtactttcgctcagagcgtgtctagcgcctacaccattgccgttgtggttttccattttctggtgccaatgatcattgtcatcttttgttatcttcgcatatggattcttgtgcttcaggtgcggcagagagtgaaacccgaccgaaagccaaagttgaagccccaggatttccggaacttcgttaccatgtttgtcgtcttcgtactgttcgcaatctgctgggcccctctgaatttcattggcctggcggtggcctcagaccccgccagtatggttcctagaatacctgagtggctgttcgtagcgagctactacatggcttactttaactcttgtcttaacgctatcatctacggactgcttaaccagaacttcaggaaggagtacaggagaatcattgtttctctttgtaccgcacgcgtattctttgtagacagctccaatgacgtggcagatcgagtcaaatggaagcctagccctctgatgactaataataacgtcgtgaaagtcgacagtgtgtaa

SEQ ID NO: 38 - DNA, 1089 bp, Artificial Sequence
  MTNR1B
atgagcgagaacggttccttcgctaattgctgcgaagcaggtggatgggccgttcgccctgggtggtccggagcaggttctgcaagaccctcaagaactccacgcccaccttgggtggcacctgctctttccgccgtgctcattgtcacaactgccgtagatgttgttgggaatcttttggtcattctctctgtactgaggaatcggaagttgcggaatgcaggcaacctcttcctcgtgtcactggcacttgctgaccttgtggtggccttctatccataccccttgatactggttgctatcttctacgatggctgggctcttggtgaggagcactgtaaggccagcgctttcgtgatggggttgtccgtcattggaagtgtttttaacattacagcaatagctatcaatcggtactgctatatttgtcacagcatggcataccatagaatctacagacgctggcatactccactccacatttgccttatatggcttctcacagttgtggccctgttgcctaactttttcgttggatccttggagtacgacccccgaatttactcatgcacctttatacaaaccgccagcacccagtatactgctgcagtcgtggtcatccatttccttctccctattgccgtcgtttcattttgctatctccggatctgggtgttggtcctgcaggctaggcggaaggcaaaacccgagtctcggctttgcctgaagccaagcgacctgcgatcttttttgaccatgttcgtcgtcttcgtgattttcgccatctgttgggctccactgaattgcatcggcctggctgttgcgatcaatccacaagaaatggcacctcaaattcccgaggggctcttcgtgaccagctaccttctggcatatttcaacagttgcctgaatgctatcgtgtacggattgctgaaccagaacttccgaagggaatataaaaggatcctgctggcactctggaaccctaggcattgtatccaagacgcatccaaggggtctcacgcggaaggcctccagagccctgcaccacccattattggtgtgcaacatcaggccgacgctttgtaa

SEQ ID NO: 39 - DNA, 1029 bp, Artificial Sequence
  PTAFR
atggagccacatgacagttctcacatggattcagagttccggtatacgctctttcccatcgtctacagcattatcttcgtcttgggcgtgattgccaatgggtacgtactctgggttttcgccagactctatccgtgcaaaaagtttaatgaaatcaaaatcttcatggtgaatctcaccatggcagacatgttgttcctcatcacactgcccctgtggattgtgtactatcagaatcagggcaattggattcttcctaaatttctgtgcaatgtcgcaggctgcctgtttttcatcaatacatattgtagcgtcgcatttctgggggtgatcacatataaccgcttccaggcagtgacgcggccaataaaaaccgcacaggcaaatacaaggaagagagggataagcctgtctctcgttatctgggtcgccattgttggggcagcgtcctacttcctgatactggatagcaccaacacagttcccgacagcgccggtagcgggaatgttaccaggtgttttgagcactacgagaagggctccgttcctgttctgataatacatatatttatagttttttcattcttcttggtgtttttgatcatattgttttgtaatctggtgatcattcgcacacttctgatgcagcccgtgcagcaacaacgcaatgctgaagtaaagcgccgggcgctttggatggtatgtacagtgctcgcagtgtttatcatatgtttcgtaccccatcatgtggtgcagctgccgtggaccttggccgaactcggcttccaggattctaagtttcaccaggctataaacgatgcccaccaagtaactctctgcctgctgagtaccaactgcgtgttggatcccgttatttattgcttcctcacaaagaagtttcgcaagcacttgacggaaaaattctactctatgcgaagcagtcggaaatgtagtagggccactaccgatacagtcaccgaggtggtggtgccgttcaatcagataccagggaacagtctgaagaattaa

SEQ ID NO: 40 - DNA, 1173 bp, Artificial Sequence
  PTGER3
atgaaagagacgcgcggctacggcggcgatgcccctttctgtactagactcaatcacagctacactgggatgtgggcccctgaacgctccgcagaggctcgcgggaacctgactcgcccgccaggaagtggggaagactgcgggtctgtcagcgtggcatttcctataacaatgctgctcactggctttgttgggaatgctctcgccatgcttctcgtgagtaggagctaccgccgaagggagtccaagcgcaagaaaagcttcctcctttgtatcgggtggctcgccctcacagacttggtaggtcagcttctgactacacctgtagtcattgtggtgtatctctcaaagcagagatgggaacatatcgacccatccggccgactttgtactttcttcggacttactatgactgtgttcgggctgagctcactgttcattgctagcgctatggccgtagagcgggccttggctatcagagcacctcattggtacgccagtcatatgaaaacccgcgccacacgagctgtcctcctcggcgtatggctggccgtgttggcgttcgcacttctgcctgtgctgggcgtgggacagtacacagtgcaatggccaggaacatggtgcttcattagcacagggagaggtgggaacggaacaagttcctctcacaactgggggaatctgttctttgcctcagcattcgctttcctcgggctgttggccctcaccgtcacattctcatgtaacctcgcaacaatcaaggcactcgtgtctcgatgtagagcaaaggcaacagcctcccagtccagtgctcaatggggccgcatcacaactgaaactgccatccagctgatggggatcatgtgcgtgttgagcgtatgctggtcaccactgctgattatgatgctgaagatgatcttcaaccaaaccagcgtcgagcactgcaagactcataccgagaaacagaaagaatgtaactttttccttatcgcggtgcggctggctagtctgaatcagattctcgacccttgggtgtatctcctgcttcgaaaaattttgctgaggaagttttgtcaaatccgctaccacacaaataattatgcatccagtagcacttccctcccgtgtcagtgtagcagcaccctgatgtggtccgaccatctcgaacggtaa

SEQ ID NO: 41 - DNA, 1149 bp, Artificial Sequence
  S1PR1
atgggaccaacatcagtcccattggttaaagcccatcgaagctccgtgagcgactacgtgaactacgacatcatcgtgaggcactataactataccggaaagctcaacatatccgccgataaagagaatagtattaaactgacctctgtcgtcttcatactgatctgttgtttcatcattctcgaaaacattttcgtgctccttaccatctggaaaaccaaaaagttccacagacctatgtactattttatcggaaatctggccctgtctgacttgttggccggggtggcctatactgcaaacttgctgctctcaggcgccactacatacaaactcacgcccgctcagtggtttctgagggagggctccatgtttgtggcgctgagcgctagcgtgttcagcctgctggctatagccatcgaacgatacattactatgctgaagatgaagctgcacaatggctctaacaattttaggctctttctccttatatccgcgtgctgggtgatttctctgattctcggggggctgcccatcatggggtggaattgcatctctgctctgtcatcatgctccactgtgcttcccctgtatcataagcactatatattgttttgtaccactgttttcaccctcttgctgctcagcattgtgattctctactgccgcatatactccttggtccggacaagatccaggcgcctgacgttccggaaaaacatctctaaggcgtctagatcatccgagaaaagcctggcgctcctgaagaccgtcatcatcgtgttgagcgtctttatcgcttgctgggcccccctgtttatcttgctcctgctggatgtagggtgtaaagtgaagacgtgcgatatcctgttccgggccgagtattttcttgtcctggcagtcctgaactccggcactaatcctatcatttataccctgaccaataaagaaatgcgccgcgccttcatcaggattatgagctgctgtaagtgtccctccggagactctgctggcaaatttaagcggccaatcatagccggcatggagttctccagatctaaatccgacaatagctcacaccctcagaaagatgaaggcgacaatcctgaaacaattatgagtagtggtaatgtgaatagctcaagctaa

SEQ ID NO: 42 - DNA, 1062 bp, Artificial Sequence
  S1PR2
atgggctcactttacagtgaatacctgaatcccaacaaggtccaagaacactacaattataccaaggaaacactggagacacaggaaacgacttcccgacaggtggcctccgcattcattgtgattctctgctgtgcaatcgtggtggagaatctgcttgtgctgatcgcagtagctaggaatagcaaatttcatagcgctatgtatctcttccttggaaatctggccgcttctgaccttcttgcgggtgttgccttcgtggcaaatacactgttgtccgggtccgttacattgcggctcacccccgtgcagtggtttgcccgcgagggctccgcctttatcactctgtccgcttccgtcttcagcctcctggcgatcgccattgagagacatgtggcaatcgcgaaagtgaaactgtacgggagtgacaagagctgcagaatgctgctgctgatcggcgcaagctggttgatcagtctggttttgggcggcctgcccatcctcgggtggaactgtctcggtcatctcgaagcatgtagcactgtgttgccactgtacgcaaaacattacgtcctgtgcgttgttactatctttagcattattctgcttgcaatcgtggcactgtacgtaaggatatactgtgtggtcagaagttctcacgctgatatggcagcacctcagaccctcgcactcctgaaaacagtcaccattgtgctgggagtgttcatagtgtgttggctgcccgcgttcagcatattgctcttggactacgcctgtcccgtgcattcctgccccatcttgtataaggcccattacttttttgctgtgtccacccttaactccctgctcaatccagttatttacacatggcgatctcgcgatctgcgacgagaagtgcttcggcccctccagtgttggaggcctggggttggagttcagggcagaagacgcggaggaactccaggtcaccacctgttgcccctgaggtctagtagtagcttggaacgcggtatgcatatgcccacttctcccaccttccttgagggcaacaccgtcgtgtaa

SEQ ID NO: 43 - DNA, 1137 bp, Artificial Sequence
  S1PR3
atggcaaccgcccttccgcccaggctccagccagtgagaggtaacgagaccctgagagagcactatcagtatgttggcaagcttgccggtagattgaaagaggcatcagagggatcaacactgacaaccgtcctctttctcgtgatatgctctttcatcgttctggaaaatctgatggtcctgattgccatttggaaaaacaacaagttccataaccgaatgtattttttcataggcaacctggccctgtgcgacctgctcgctggtatcgcctataaagtgaacatcctgatgagcggaaaaaagacctttagtctgagcccaacagtgtggttcctgagagaagggagcatgttcgtggcgctgggggcgagtacctgtagtcttctcgccatcgcgattgaaagacacttgaccatgattaagatgaggccatatgacgcaaacaagcgacatcgggtgtttctccttatcgggatgtgttggctgatcgcattcactctcggggctctgcctatactggggtggaactgcctgcacaatctcccagattgctctacaatcctcccactgtatagtaagaaatatattgcattttgcatctccatcttcactgccatcctggttaccatcgtcatcctgtacgcgcggatctactttctggtcaaaagtagcagtaggaaggtggctaatcacaataattctgaacggtctatggccttgctgcggaccgtggtgatagtcgtgtcagtgttcatagcctgttggtctccgctgtttatcctctttctgatagatgtcgcgtgccgagttcaggcttgccccatcctcttcaaggctcagtggtttatagtccttgcagtcctcaattccgctatgaatcccgtcatttacaccctggcctcaaaggagatgagacgagctttctttaggctggtgtgcaattgcctcgtcaggggaagaggagcgcgggcatcccccatccaacctgctctggatccgtctcggtcaaagtcaagctcaagcaacaactcttctcactctcccaaggtaaaagaagatctgccccatacggcacctagcagctgtatcatggacaaaaatgcagccctccaaaacggcatattctgtaactaa

SEQ ID NO: 44 - DNA, 1095 bp, Artificial Sequence
  SSTR5
atggaaccactcttccctgccagcactcctagctggaacgcttcttctcccggcgcagccagcggtggtggcgacaatagaacgctggtgggccccgctccttcagccggagcccgggctgtactggtcccagtcctgtacctgctggtctgcgccgctgggttgggcggcaacacactggtgatttacgtagtgttgcggtttgcaaagatgaaaactgtcacgaatatctatattctcaacttggcagtagccgacgtgctgtatatgctcggattgccgttcctggccactcagaatgcggcatctttctggcctttcggacctgtgctgtgcaggctggttatgacccttgatggcgtcaatcagtttacctccgttttttgcctgacagtgatgagtgttgatcggtacctcgccgtggtccatccgctctcatctgcccgatggaggaggcctagggtggcgaaactggcaagtgctgctgcatgggtgctgtccctctgtatgagcctccctctgctcgtgttcgcagacgtgcaagagggagggacatgtaatgcttcctggcctgagcccgtaggactttggggcgctgtgtttattatttacaccgctgttctgggattcttcgcccctctgctggtcatttgcctttgctatctgctgatagtcgtaaaagttcgggccgccggggtcagagttggctgtgtgaggcgccggtccgaacgcaaggtgaccagaatggtgttggtggtggtgctggtgttcgcgggctgctggctgcctttcttcacagtgaatatcgtcaatctcgccgtagcactgccgcaggaaccagcatcagcaggactctacttcttcgtagttatattgtcttacgccaactcctgtgccaacccagtcctctatgggttcctctctgataacttccggcaaagctttcagaaagtactttgccttagaaaggggagcggagcgaaggacgcagatgcaaccgagcctcgaccagacaggattcgacagcagcaagaggccacaccacccgcacaccgggccgcagcaaacgggttgatgcagaccagcaaactgtaa

SEQ ID NO: 45 - DNA, 1005 bp, Artificial Sequence
  SUCNR1
atgctgggaattatggcctggaatgccacctgtaagaattggctggctgcagaggctgccctggaaaaatattacctgtccattttctatgggatcgagtttgtagtgggtgttctgggtaatactattgtggtgtacggatacatcttctccctgaaaaattggaattcttctaatatttatctgttcaatctttccgtgtccgatctggctttcctgtgcaccttgccgatgctcatcagatcatatgccaatgggaattggatctatggcgacgtcctctgtatctccaatagatacgtcctgcacgccaacctgtatacctcaattttgttcttgacgttcataagtatcgaccgctacctcatcataaagtacccctttcgcgaacacctgcttcagaaaaaagaatttgccattctcattagcttggctatctgggttcttgtgacccttgaactcctcccgattctgcccctcatcaaccccgtgatcacagataatggcactacctgtaacgactttgcttccagtggggacccgaattacaatctgatctacagtatgtgtctcacactcctcggatttctgattcctctgttcgtgatgtgtttcttttattacaagatagctctgtttttgaagcagagaaacagacaggtggctaccgctctcccactggagaagccactgaacctggtcatcatggcggttgtgatcttctctgttctttttactccataccacgtgatgagaaacgtcaggatagccagtcggctgggctcatggaagcagtaccaatgcacccaggtggtgataaacagcttctatattgttactcggccactggctttcctgaattctgttatcaaccctgtgttctattttctgctgggcgaccactttagggatatgctgatgaaccagttgcggcacaattttaagtccctgacatccttttcccggtgggcccacgaactgctcctgtcttttagggagaagtaa

SEQ ID NO: 46 - DNA, 11251 bp, Artificial Sequence
  pML104
aacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctccaccgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcagaatcgatgcatttaaatagcaagttaaaataaggctagtccgttatcaacttgaaaaagtggcaccgagtcggtggtgctttttttgttttttatgtcttcgagtcatgtaattagttataagcatgtgagctaagacactgtaattgccaatctaaacgataccacggccgctctagagaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcctcgagagtctaggatccggaactacctaccttgcgctttttcttgggagctccctcatcccctccgagctgtgagaggtcaatccgagtttcgtaaagtccggtgatgctctggtggatgagggtggcgtccagcacttctttggtggaggtatagcgtttccggtcgatggtggtgtcgaagtacttgaatgcggcaggggcaccaagattggtgagggtgaacaggtggatgatattctcagcctgctcccgaattggtttgtctctgtgcttattgtatgcgctaaggactttatcgaggttggcatcagcgagaatgacgcgtttagaaaactcggaaatctgctcaatgatctcgtccagataatgcttatgctgctccacaaacagttgcttttgctcgttgtcctcggggctccccttcagcttctcgtaatggcttgcaaggtagaggaaattcacgtacttggaggggagagccagctcattacccttctgaagttcgccagcggaggcgagcatccgcttccgcccattctccagttcgaacaggctatactttgggagcttgatgataaggtccttcttgacttccttgtaacctttggcttcgagaaagtcaattgggttcttctcgaaggagctgcgctccataatggtgatacccagcagttccttcacgctcttgagcttcttagactttcctttttccaccttagccacgaccaggacagaataagccacagttggtgaatcgaatccgccgtacttcttagggtcccaatccttcttccttgcaatgagcttgtcggagttgcgcttggggaggattgattccttagaaaagcctccggtttgcacctcggttttcttcacgatgttcacttgaggcatagaaagcactttgcgcacggtagcgaaatctctgcccttgtcccacacgatttcgccagtctccccattagtctcgatgagcggcctcttccggatctcaccgtttgcaagggtgatttcggtcttgaagaaattcatgatgttgctgtagaagaagtactttgcggtggcctttccgatctcctgctcagatttggcaatcatcttgcgcacgtcgtagaccttgtagtctccgtacacgaactcgctttccagctttgggtacttcttgattagggcggttcccaccacggcattcagatatgcgtcatgagcatgatgatagttattgatctcccgcactttgtagaattgaaagtcctttctgaaatcgctgaccagcttggacttcagggtaatcaccttcacctcgcggataagtttgtcgttttcgtcgtacttggtattcatgcgtgagtcaaggatttgggcgacatgtttagtaatctgccgagtctccacaagttgccgtttaatgaatcctgctttgtccagttcgctcagtccgcccctctcagccttagtgagattgtcgaacttccgctgagtgattagctttgcgttcagaagctggcgccaatagttcttcatctttttcacgacttcctctgagggcacgttatctgactttccccggttcttgtctgagcgagtcagcaccttattgtcaattgagtcgtctttgaggaagctctgagggacgatgtggtccacgtcgtagtctgagagcctgttgatatcaagctcttggtccacatacatatcgcgtccattttgaaggtagtacagatagagcttctcattctgaagctgggtgttttccaccgggtgctctttaaggatctgtgacccgagctccttaatcccttcctcaattcttttcatccgctccctgctgttcttctgtcccttctgggtagtctggttttccctggccatctcgatgacaatgttctctggtttatggcgacccatcaccttgaccagctcgtccacgactttcacggtctggagaatacccttcttaatagcgggtgaaccagccagatttgcgatatgctcgtgcagtgagtcaccttgtccagacacttgggccttctggatgtcctccttaaaggtcagggaatcatcatggataagttgcatgaagttgcgattagcgaatccgtcgctcttaaggaaatcaagaatggtctttccgctctgtttgtcccggattccgttgatgagtttgcggctaaggcgtccccatccggtatatcttctccgtttaagttgcttcatcactttatcgtcgaacagatgggcgtaggtcttaagcctctcctcgatcatttcccggtcctcgaacagagtgagagtcaggacaatgtcctcaaggatgtcctcgttctcctcattgtccagaaaatccttgtccttgatgatcttaagcagatcatggtaggtacccaggcttgcgttgaaccgatcttcgaccccgctaatctccacagagtcgaagcattcaattttcttgaaatagtcctccttcagctgcttcacggtcacctttctattggtcttgaacagaaggtccacaattgccttcttttgttcgcctgacaggaatgcgggcttcctcatcccttcggtgacatatttcaccttggtcagctcgttgtacacggtaaagtactcgtagagcagggaatgtttcggcaggaccttctcattggggaggttcttatcgaagttagtcatccgttcgatgaatgactgagcgctggctcccttgtccaccacctcttcgaagttccagggagtgatagtttcttctgactttctggtcatccaagcaaaccggctatttcctctggcgaggggtcccacgtagtaggggatgcggaaagtgagaattttctcaatcttctccctgttgtccttaagaaaggggtagaagtcctcttggcgccgaaggatggcgtgaagctccccaagatggatctggtggggaatgctcccattgtcgaaagtgcgctgcttccgcagcagatcctcccgattcagtttcacgaggagctcttcggtgccgtccatcttctcaaggataggtttgataaacttataaaattcctcttgggaggcacctccgtcgatgtagccggcgtatccattctttgactggtcaaagaagatttccttgtacttctccggaagctgctgccggacgagtgccttaagcagggtcaggtcctggtggtgctcatcataccgcttaatcattgaagctgaaagaggggccttggtgatttcagtgttcacgcggagaatgtcagacagcaggatggcgtcagaaagattcttagcagcaaggaaaaggtcggcgtattggtcaccgatctgggccaggaggttgtcgagatcgtcatcataggtgtccttggacagttgcagcttggcatcctctgccaggtcgaaattagacttgaaattgggagtcagcccaagtgaaagagcaatcaggttcccgaacagtccatttttcttttcacccggcagctgagcgataagattctcaagtctgcggctctttgacagccgggctgacagaatggccttggcatcgactccgcttgcattgatggggttctcttcaaagagctggttgtaagtctgcaccagctggatgaacagtttatccacatcgctattgtcaggattgaggtcgccctcgatcaggaagtggcctctgaacttaatcatgtgggccagggccaggtagataagccggaggtctgccttgtcggttgagtccaccagctttttgcgaagatggtagatggttgggtatttctcgtgataggccacctcgtcgacgatattaccgaagatgggatgccgttcatgcttcttatcctcttccacgaggaatgattcttccagcctatggaagaagctgtcgtcgacctttgccatttcgttggagaatatctcttgaaggtaacaaatccggttcttcctcctggtgtaccgccgtctagcagtccgcttgaggcgggttgcttcagcggtctcccctgaatcaaagaggagagctccgatcagatttttcttgatggaatgccggtcggtgttcccgagcaccttgaacttcttagagggcaccttgtactcgtcagtgatgacggcccacccgacgctattagtcccgatgtccagtccgatagaatacttcttgtccatggtggcgagatctaattcgatccatatcagatcttttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgactagtagactgaattcgatatcaagcttatcgataccgtcgacctcgagggggggcccggtacccaattcgccctatagtgagtcgtattacgcgcgctcactggccgtcgttttac

SEQ ID NO: 47 - DNA, 12366 bp, Artificial Sequence
  pML107
gacgaaagggcctcgtgatacgcctatttttataggttaatgtcatgataataatggtttcttagatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcagaatcgatgcatttaaatagcaagttaaaataaggctagtccgttatcaacttgaaaaagtggcaccgagtcggtggtgctttttttgttttttatgtcttcgagtcatgtaattagttataagcatgtgagctaagacactgtaattgccaatctaaacgataccacggccgctctagagaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcctcgagagtctaggatccggaactacctaccttgcgctttttcttgggagctccctcatcccctccgagctgtgagaggtcaatccgagtttcgtaaagtccggtgatgctctggtggatgagggtggcgtccagcacttctttggtggaggtatagcgtttccggtcgatggtggtgtcgaagtacttgaatgcggcaggggcaccaagattggtgagggtgaacaggtggatgatattctcagcctgctcccgaattggtttgtctctgtgcttattgtatgcgctaaggactttatcgaggttggcatcagcgagaatgacgcgtttagaaaactcggaaatctgctcaatgatctcgtccagataatgcttatgctgctccacaaacagttgcttttgctcgttgtcctcggggctccccttcagcttctcgtaatggcttgcaaggtagaggaaattcacgtacttggaggggagagccagctcattacccttctgaagttcgccagcggaggcgagcatccgcttccgcccattctccagttcgaacaggctatactttgggagcttgatgataaggtccttcttgacttccttgtaacctttggcttcgagaaagtcaattgggttcttctcgaaggagctgcgctccataatggtgatacccagcagttccttcacgctcttgagcttcttagactttcctttttccaccttagccacgaccaggacagaataagccacagttggtgaatcgaatccgccgtacttcttagggtcccaatccttcttccttgcaatgagcttgtcggagttgcgcttggggaggattgattccttagaaaagcctccggtttgcacctcggttttcttcacgatgttcacttgaggcatagaaagcactttgcgcacggtagcgaaatctctgcccttgtcccacacgatttcgccagtctccccattagtctcgatgagcggcctcttccggatctcaccgtttgcaagggtgatttcggtcttgaagaaattcatgatgttgctgtagaagaagtactttgcggtggcctttccgatctcctgctcagatttggcaatcatcttgcgcacgtcgtagaccttgtagtctccgtacacgaactcgctttccagctttgggtacttcttgattagggcggttcccaccacggcattcagatatgcgtcatgagcatgatgatagttattgatctcccgcactttgtagaattgaaagtcctttctgaaatcgctgaccagcttggacttcagggtaatcaccttcacctcgcggataagtttgtcgttttcgtcgtacttggtattcatgcgtgagtcaaggatttgggcgacatgtttagtaatctgccgagtctccacaagttgccgtttaatgaatcctgctttgtccagttcgctcagtccgcccctctcagccttagtgagattgtcgaacttccgctgagtgattagctttgcgttcagaagctggcgccaatagttcttcatctttttcacgacttcctctgagggcacgttatctgactttccccggttcttgtctgagcgagtcagcaccttattgtcaattgagtcgtctttgaggaagctctgagggacgatgtggtccacgtcgtagtctgagagcctgttgatatcaagctcttggtccacatacatatcgcgtccattttgaaggtagtacagatagagcttctcattctgaagctgggtgttttccaccgggtgctctttaaggatctgtgacccgagctccttaatcccttcctcaattcttttcatccgctccctgctgttcttctgtcccttctgggtagtctggttttccctggccatctcgatgacaatgttctctggtttatggcgacccatcaccttgaccagctcgtccacgactttcacggtctggagaatacccttcttaatagcgggtgaaccagccagatttgcgatatgctcgtgcagtgagtcaccttgtccagacacttgggccttctggatgtcctccttaaaggtcagggaatcatcatggataagttgcatgaagttgcgattagcgaatccgtcgctcttaaggaaatcaagaatggtctttccgctctgtttgtcccggattccgttgatgagtttgcggctaaggcgtccccatccggtatatcttctccgtttaagttgcttcatcactttatcgtcgaacagatgggcgtaggtcttaagcctctcctcgatcatttcccggtcctcgaacagagtgagagtcaggacaatgtcctcaaggatgtcctcgttctcctcattgtccagaaaatccttgtccttgatgatcttaagcagatcatggtaggtacccaggcttgcgttgaaccgatcttcgaccccgctaatctccacagagtcgaagcattcaattttcttgaaatagtcctccttcagctgcttcacggtcacctttctattggtcttgaacagaaggtccacaattgccttcttttgttcgcctgacaggaatgcgggcttcctcatcccttcggtgacatatttcaccttggtcagctcgttgtacacggtaaagtactcgtagagcagggaatgtttcggcaggaccttctcattggggaggttcttatcgaagttagtcatccgttcgatgaatgactgagcgctggctcccttgtccaccacctcttcgaagttccagggagtgatagtttcttctgactttctggtcatccaagcaaaccggctatttcctctggcgaggggtcccacgtagtaggggatgcggaaagtgagaattttctcaatcttctccctgttgtccttaagaaaggggtagaagtcctcttggcgccgaaggatggcgtgaagctccccaagatggatctggtggggaatgctcccattgtcgaaagtgcgctgcttccgcagcagatcctcccgattcagtttcacgaggagctcttcggtgccgtccatcttctcaaggataggtttgataaacttataaaattcctcttgggaggcacctccgtcgatgtagccggcgtatccattctttgactggtcaaagaagatttccttgtacttctccggaagctgctgccggacgagtgccttaagcagggtcaggtcctggtggtgctcatcataccgcttaatcattgaagctgaaagaggggccttggtgatttcagtgttcacgcggagaatgtcagacagcaggatggcgtcagaaagattcttagcagcaaggaaaaggtcggcgtattggtcaccgatctgggccaggaggttgtcgagatcgtcatcataggtgtccttggacagttgcagcttggcatcctctgccaggtcgaaattagacttgaaattgggagtcagcccaagtgaaagagcaatcaggttcccgaacagtccatttttcttttcacccggcagctgagcgataagattctcaagtctgcggctctttgacagccgggctgacagaatggccttggcatcgactccgcttgcattgatggggttctcttcaaagagctggttgtaagtctgcaccagctggatgaacagtttatccacatcgctattgtcaggattgaggtcgccctcgatcaggaagtggcctctgaacttaatcatgtgggccagggccaggtagataagccggaggtctgccttgtcggttgagtccaccagctttttgcgaagatggtagatggttgggtatttctcgtgataggccacctcgtcgacgatattaccgaagatgggatgccgttcatgcttcttatcctcttccacgaggaatgattcttccagcctatggaagaagctgtcgtcgacctttgccatttcgttggagaatatctcttgaaggtaacaaatccggttcttcctcctggtgtaccgccgtctagcagtccgcttgaggcgggttgcttcagcggtctcccctgaatcaaagaggagagctccgatcagatttttcttgatggaatgccggtcggtgttcccgagcaccttgaacttcttagagggcaccttgtactcgtcagtgatgacggcccacccgacgctattagtcccgatgtccagtccgatagaatacttcttgtccatggtggcgagatctaattcgatccatatcagatcttttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgactagtagactgaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatcgaccggtcgaggagaacttctagtatatctacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagcgccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatagatgctacaggtgttccacttccagatgaggcgctggaagcctccaagaaggctgatgccgttttgttaggtgctgtgggtggtcctaaatggggtacaggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaatttcctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaatttatatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttaacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 48 - DNA, 5186 bp, Artificial Sequence
  pT040
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcttagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgattttaggcaaaagttacaatttctggctcgtgtaatatatgtatgctaaagtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagttatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcagaatcgatgcatttaaatagcaagttaaaataaggctagtccgttatcaacttgaaaaagtggcaccgagtcggtggtgctttttttgttttttatgtcttcgagtcatgtaattagttataagcatgtgagctaagacactgtaattgccaatctaaacgataccacggccgctctagaactagtggatcccccccgctcgagcgggggctgcaggaattcgatatcaagcttatcgataccgtcgacctcgatcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 49 - DNA, 12384 bp, Artificial Sequence
  pML107
gacgaaagggcctcgtgatacgcctatttttataggttaatgtcatgataataatggtttcttagatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttttcaaacgaaactcttgtgttttagagctagaaatagcaagttaaaataaggctagtccgttatcaacttgaaaaagtggcaccgagtcggtggtgctttttttgttttttatgtcttcgagtcatgtaattagttataagcatgtgagctaagacactgtaattgccaatctaaacgataccacggccgctctagagaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcctcgagagtctaggatccggaactacctaccttgcgctttttcttgggagctccctcatcccctccgagctgtgagaggtcaatccgagtttcgtaaagtccggtgatgctctggtggatgagggtggcgtccagcacttctttggtggaggtatagcgtttccggtcgatggtggtgtcgaagtacttgaatgcggcaggggcaccaagattggtgagggtgaacaggtggatgatattctcagcctgctcccgaattggtttgtctctgtgcttattgtatgcgctaaggactttatcgaggttggcatcagcgagaatgacgcgtttagaaaactcggaaatctgctcaatgatctcgtccagataatgcttatgctgctccacaaacagttgcttttgctcgttgtcctcggggctccccttcagcttctcgtaatggcttgcaaggtagaggaaattcacgtacttggaggggagagccagctcattacccttctgaagttcgccagcggaggcgagcatccgcttccgcccattctccagttcgaacaggctatactttgggagcttgatgataaggtccttcttgacttccttgtaacctttggcttcgagaaagtcaattgggttcttctcgaaggagctgcgctccataatggtgatacccagcagttccttcacgctcttgagcttcttagactttcctttttccaccttagccacgaccaggacagaataagccacagttggtgaatcgaatccgccgtacttcttagggtcccaatccttcttccttgcaatgagcttgtcggagttgcgcttggggaggattgattccttagaaaagcctccggtttgcacctcggttttcttcacgatgttcacttgaggcatagaaagcactttgcgcacggtagcgaaatctctgcccttgtcccacacgatttcgccagtctccccattagtctcgatgagcggcctcttccggatctcaccgtttgcaagggtgatttcggtcttgaagaaattcatgatgttgctgtagaagaagtactttgcggtggcctttccgatctcctgctcagatttggcaatcatcttgcgcacgtcgtagaccttgtagtctccgtacacgaactcgctttccagctttgggtacttcttgattagggcggttcccaccacggcattcagatatgcgtcatgagcatgatgatagttattgatctcccgcactttgtagaattgaaagtcctttctgaaatcgctgaccagcttggacttcagggtaatcaccttcacctcgcggataagtttgtcgttttcgtcgtacttggtattcatgcgtgagtcaaggatttgggcgacatgtttagtaatctgccgagtctccacaagttgccgtttaatgaatcctgctttgtccagttcgctcagtccgcccctctcagccttagtgagattgtcgaacttccgctgagtgattagctttgcgttcagaagctggcgccaatagttcttcatctttttcacgacttcctctgagggcacgttatctgactttccccggttcttgtctgagcgagtcagcaccttattgtcaattgagtcgtctttgaggaagctctgagggacgatgtggtccacgtcgtagtctgagagcctgttgatatcaagctcttggtccacatacatatcgcgtccattttgaaggtagtacagatagagcttctcattctgaagctgggtgttttccaccgggtgctctttaaggatctgtgacccgagctccttaatcccttcctcaattcttttcatccgctccctgctgttcttctgtcccttctgggtagtctggttttccctggccatctcgatgacaatgttctctggtttatggcgacccatcaccttgaccagctcgtccacgactttcacggtctggagaatacccttcttaatagcgggtgaaccagccagatttgcgatatgctcgtgcagtgagtcaccttgtccagacacttgggccttctggatgtcctccttaaaggtcagggaatcatcatggataagttgcatgaagttgcgattagcgaatccgtcgctcttaaggaaatcaagaatggtctttccgctctgtttgtcccggattccgttgatgagtttgcggctaaggcgtccccatccggtatatcttctccgtttaagttgcttcatcactttatcgtcgaacagatgggcgtaggtcttaagcctctcctcgatcatttcccggtcctcgaacagagtgagagtcaggacaatgtcctcaaggatgtcctcgttctcctcattgtccagaaaatccttgtccttgatgatcttaagcagatcatggtaggtacccaggcttgcgttgaaccgatcttcgaccccgctaatctccacagagtcgaagcattcaattttcttgaaatagtcctccttcagctgcttcacggtcacctttctattggtcttgaacagaaggtccacaattgccttcttttgttcgcctgacaggaatgcgggcttcctcatcccttcggtgacatatttcaccttggtcagctcgttgtacacggtaaagtactcgtagagcagggaatgtttcggcaggaccttctcattggggaggttcttatcgaagttagtcatccgttcgatgaatgactgagcgctggctcccttgtccaccacctcttcgaagttccagggagtgatagtttcttctgactttctggtcatccaagcaaaccggctatttcctctggcgaggggtcccacgtagtaggggatgcggaaagtgagaattttctcaatcttctccctgttgtccttaagaaaggggtagaagtcctcttggcgccgaaggatggcgtgaagctccccaagatggatctggtggggaatgctcccattgtcgaaagtgcgctgcttccgcagcagatcctcccgattcagtttcacgaggagctcttcggtgccgtccatcttctcaaggataggtttgataaacttataaaattcctcttgggaggcacctccgtcgatgtagccggcgtatccattctttgactggtcaaagaagatttccttgtacttctccggaagctgctgccggacgagtgccttaagcagggtcaggtcctggtggtgctcatcataccgcttaatcattgaagctgaaagaggggccttggtgatttcagtgttcacgcggagaatgtcagacagcaggatggcgtcagaaagattcttagcagcaaggaaaaggtcggcgtattggtcaccgatctgggccaggaggttgtcgagatcgtcatcataggtgtccttggacagttgcagcttggcatcctctgccaggtcgaaattagacttgaaattgggagtcagcccaagtgaaagagcaatcaggttcccgaacagtccatttttcttttcacccggcagctgagcgataagattctcaagtctgcggctctttgacagccgggctgacagaatggccttggcatcgactccgcttgcattgatggggttctcttcaaagagctggttgtaagtctgcaccagctggatgaacagtttatccacatcgctattgtcaggattgaggtcgccctcgatcaggaagtggcctctgaacttaatcatgtgggccagggccaggtagataagccggaggtctgccttgtcggttgagtccaccagctttttgcgaagatggtagatggttgggtatttctcgtgataggccacctcgtcgacgatattaccgaagatgggatgccgttcatgcttcttatcctcttccacgaggaatgattcttccagcctatggaagaagctgtcgtcgacctttgccatttcgttggagaatatctcttgaaggtaacaaatccggttcttcctcctggtgtaccgccgtctagcagtccgcttgaggcgggttgcttcagcggtctcccctgaatcaaagaggagagctccgatcagatttttcttgatggaatgccggtcggtgttcccgagcaccttgaacttcttagagggcaccttgtactcgtcagtgatgacggcccacccgacgctattagtcccgatgtccagtccgatagaatacttcttgtccatggtggcgagatctaattcgatccatatcagatcttttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgactagtagactgaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatcgaccggtcgaggagaacttctagtatatctacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagcgccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatagatgctacaggtgttccacttccagatgaggcgctggaagcctccaagaaggctgatgccgttttgttaggtgctgtgggtggtcctaaatggggtacaggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaatttcctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaatttatatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttaacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 50 - DNA, 5204 bp, Artificial Sequence
  pT040 FAR12442
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcttagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgattttaggcaaaagttacaatttctggctcgtgtaatatatgtatgctaaagtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagttatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcgaattctttgctgctttaccgttttagagctagaaatagcaagttaaaataaggctagtccgttatcaacttgaaaaagtggcaccgagtcggtggtgctttttttgttttttatgtcttcgagtcatgtaattagttataagcatgtgagctaagacactgtaattgccaatctaaacgataccacggccgctctagaactagtggatcccccccgctcgagcgggggctgcaggaattcgatatcaagcttatcgataccgtcgacctcgatcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 51 - DNA, 12384 bp, Artificial Sequence
  pML107 STE237
gacgaaagggcctcgtgatacgcctatttttataggttaatgtcatgataataatggtttcttagatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctatgatccaacgtataatccgttttagagctagaaatagcaagttaaaataaggctagtccgttatcaacttgaaaaagtggcaccgagtcggtggtgctttttttgttttttatgtcttcgagtcatgtaattagttataagcatgtgagctaagacactgtaattgccaatctaaacgataccacggccgctctagagaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcctcgagagtctaggatccggaactacctaccttgcgctttttcttgggagctccctcatcccctccgagctgtgagaggtcaatccgagtttcgtaaagtccggtgatgctctggtggatgagggtggcgtccagcacttctttggtggaggtatagcgtttccggtcgatggtggtgtcgaagtacttgaatgcggcaggggcaccaagattggtgagggtgaacaggtggatgatattctcagcctgctcccgaattggtttgtctctgtgcttattgtatgcgctaaggactttatcgaggttggcatcagcgagaatgacgcgtttagaaaactcggaaatctgctcaatgatctcgtccagataatgcttatgctgctccacaaacagttgcttttgctcgttgtcctcggggctccccttcagcttctcgtaatggcttgcaaggtagaggaaattcacgtacttggaggggagagccagctcattacccttctgaagttcgccagcggaggcgagcatccgcttccgcccattctccagttcgaacaggctatactttgggagcttgatgataaggtccttcttgacttccttgtaacctttggcttcgagaaagtcaattgggttcttctcgaaggagctgcgctccataatggtgatacccagcagttccttcacgctcttgagcttcttagactttcctttttccaccttagccacgaccaggacagaataagccacagttggtgaatcgaatccgccgtacttcttagggtcccaatccttcttccttgcaatgagcttgtcggagttgcgcttggggaggattgattccttagaaaagcctccggtttgcacctcggttttcttcacgatgttcacttgaggcatagaaagcactttgcgcacggtagcgaaatctctgcccttgtcccacacgatttcgccagtctccccattagtctcgatgagcggcctcttccggatctcaccgtttgcaagggtgatttcggtcttgaagaaattcatgatgttgctgtagaagaagtactttgcggtggcctttccgatctcctgctcagatttggcaatcatcttgcgcacgtcgtagaccttgtagtctccgtacacgaactcgctttccagctttgggtacttcttgattagggcggttcccaccacggcattcagatatgcgtcatgagcatgatgatagttattgatctcccgcactttgtagaattgaaagtcctttctgaaatcgctgaccagcttggacttcagggtaatcaccttcacctcgcggataagtttgtcgttttcgtcgtacttggtattcatgcgtgagtcaaggatttgggcgacatgtttagtaatctgccgagtctccacaagttgccgtttaatgaatcctgctttgtccagttcgctcagtccgcccctctcagccttagtgagattgtcgaacttccgctgagtgattagctttgcgttcagaagctggcgccaatagttcttcatctttttcacgacttcctctgagggcacgttatctgactttccccggttcttgtctgagcgagtcagcaccttattgtcaattgagtcgtctttgaggaagctctgagggacgatgtggtccacgtcgtagtctgagagcctgttgatatcaagctcttggtccacatacatatcgcgtccattttgaaggtagtacagatagagcttctcattctgaagctgggtgttttccaccgggtgctctttaaggatctgtgacccgagctccttaatcccttcctcaattcttttcatccgctccctgctgttcttctgtcccttctgggtagtctggttttccctggccatctcgatgacaatgttctctggtttatggcgacccatcaccttgaccagctcgtccacgactttcacggtctggagaatacccttcttaatagcgggtgaaccagccagatttgcgatatgctcgtgcagtgagtcaccttgtccagacacttgggccttctggatgtcctccttaaaggtcagggaatcatcatggataagttgcatgaagttgcgattagcgaatccgtcgctcttaaggaaatcaagaatggtctttccgctctgtttgtcccggattccgttgatgagtttgcggctaaggcgtccccatccggtatatcttctccgtttaagttgcttcatcactttatcgtcgaacagatgggcgtaggtcttaagcctctcctcgatcatttcccggtcctcgaacagagtgagagtcaggacaatgtcctcaaggatgtcctcgttctcctcattgtccagaaaatccttgtccttgatgatcttaagcagatcatggtaggtacccaggcttgcgttgaaccgatcttcgaccccgctaatctccacagagtcgaagcattcaattttcttgaaatagtcctccttcagctgcttcacggtcacctttctattggtcttgaacagaaggtccacaattgccttcttttgttcgcctgacaggaatgcgggcttcctcatcccttcggtgacatatttcaccttggtcagctcgttgtacacggtaaagtactcgtagagcagggaatgtttcggcaggaccttctcattggggaggttcttatcgaagttagtcatccgttcgatgaatgactgagcgctggctcccttgtccaccacctcttcgaagttccagggagtgatagtttcttctgactttctggtcatccaagcaaaccggctatttcctctggcgaggggtcccacgtagtaggggatgcggaaagtgagaattttctcaatcttctccctgttgtccttaagaaaggggtagaagtcctcttggcgccgaaggatggcgtgaagctccccaagatggatctggtggggaatgctcccattgtcgaaagtgcgctgcttccgcagcagatcctcccgattcagtttcacgaggagctcttcggtgccgtccatcttctcaaggataggtttgataaacttataaaattcctcttgggaggcacctccgtcgatgtagccggcgtatccattctttgactggtcaaagaagatttccttgtacttctccggaagctgctgccggacgagtgccttaagcagggtcaggtcctggtggtgctcatcataccgcttaatcattgaagctgaaagaggggccttggtgatttcagtgttcacgcggagaatgtcagacagcaggatggcgtcagaaagattcttagcagcaaggaaaaggtcggcgtattggtcaccgatctgggccaggaggttgtcgagatcgtcatcataggtgtccttggacagttgcagcttggcatcctctgccaggtcgaaattagacttgaaattgggagtcagcccaagtgaaagagcaatcaggttcccgaacagtccatttttcttttcacccggcagctgagcgataagattctcaagtctgcggctctttgacagccgggctgacagaatggccttggcatcgactccgcttgcattgatggggttctcttcaaagagctggttgtaagtctgcaccagctggatgaacagtttatccacatcgctattgtcaggattgaggtcgccctcgatcaggaagtggcctctgaacttaatcatgtgggccagggccaggtagataagccggaggtctgccttgtcggttgagtccaccagctttttgcgaagatggtagatggttgggtatttctcgtgataggccacctcgtcgacgatattaccgaagatgggatgccgttcatgcttcttatcctcttccacgaggaatgattcttccagcctatggaagaagctgtcgtcgacctttgccatttcgttggagaatatctcttgaaggtaacaaatccggttcttcctcctggtgtaccgccgtctagcagtccgcttgaggcgggttgcttcagcggtctcccctgaatcaaagaggagagctccgatcagatttttcttgatggaatgccggtcggtgttcccgagcaccttgaacttcttagagggcaccttgtactcgtcagtgatgacggcccacccgacgctattagtcccgatgtccagtccgatagaatacttcttgtccatggtggcgagatctaattcgatccatatcagatcttttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgactagtagactgaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatcgaccggtcgaggagaacttctagtatatctacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagcgccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatagatgctacaggtgttccacttccagatgaggcgctggaagcctccaagaaggctgatgccgttttgttaggtgctgtgggtggtcctaaatggggtacaggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaatttcctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaatttatatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttaacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 52 - DNA, 5204 bp, Artificial Sequence
  pT040 STE21259
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcttagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgattttaggcaaaagttacaatttctggctcgtgtaatatatgtatgctaaagtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagttatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcatcttcagtccagaactttcgttttagagctagaaatagcaagttaaaataaggctagtccgttatcaacttgaaaaagtggcaccgagtcggtggtgctttttttgttttttatgtcttcgagtcatgtaattagttataagcatgtgagctaagacactgtaattgccaatctaaacgataccacggccgctctagaactagtggatcccccccgctcgagcgggggctgcaggaattcgatatcaagcttatcgataccgtcgacctcgatcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 53 - DNA, 12384 bp, Artificial Sequence
  pML107 SST2-19
gacgaaagggcctcgtgatacgcctatttttataggttaatgtcatgataataatggtttcttagatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttaaagatagagttgtaagagttttagagctagaaatagcaagttaaaataaggctagtccgttatcaacttgaaaaagtggcaccgagtcggtggtgctttttttgttttttatgtcttcgagtcatgtaattagttataagcatgtgagctaagacactgtaattgccaatctaaacgataccacggccgctctagagaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcctcgagagtctaggatccggaactacctaccttgcgctttttcttgggagctccctcatcccctccgagctgtgagaggtcaatccgagtttcgtaaagtccggtgatgctctggtggatgagggtggcgtccagcacttctttggtggaggtatagcgtttccggtcgatggtggtgtcgaagtacttgaatgcggcaggggcaccaagattggtgagggtgaacaggtggatgatattctcagcctgctcccgaattggtttgtctctgtgcttattgtatgcgctaaggactttatcgaggttggcatcagcgagaatgacgcgtttagaaaactcggaaatctgctcaatgatctcgtccagataatgcttatgctgctccacaaacagttgcttttgctcgttgtcctcggggctccccttcagcttctcgtaatggcttgcaaggtagaggaaattcacgtacttggaggggagagccagctcattacccttctgaagttcgccagcggaggcgagcatccgcttccgcccattctccagttcgaacaggctatactttgggagcttgatgataaggtccttcttgacttccttgtaacctttggcttcgagaaagtcaattgggttcttctcgaaggagctgcgctccataatggtgatacccagcagttccttcacgctcttgagcttcttagactttcctttttccaccttagccacgaccaggacagaataagccacagttggtgaatcgaatccgccgtacttcttagggtcccaatccttcttccttgcaatgagcttgtcggagttgcgcttggggaggattgattccttagaaaagcctccggtttgcacctcggttttcttcacgatgttcacttgaggcatagaaagcactttgcgcacggtagcgaaatctctgcccttgtcccacacgatttcgccagtctccccattagtctcgatgagcggcctcttccggatctcaccgtttgcaagggtgatttcggtcttgaagaaattcatgatgttgctgtagaagaagtactttgcggtggcctttccgatctcctgctcagatttggcaatcatcttgcgcacgtcgtagaccttgtagtctccgtacacgaactcgctttccagctttgggtacttcttgattagggcggttcccaccacggcattcagatatgcgtcatgagcatgatgatagttattgatctcccgcactttgtagaattgaaagtcctttctgaaatcgctgaccagcttggacttcagggtaatcaccttcacctcgcggataagtttgtcgttttcgtcgtacttggtattcatgcgtgagtcaaggatttgggcgacatgtttagtaatctgccgagtctccacaagttgccgtttaatgaatcctgctttgtccagttcgctcagtccgcccctctcagccttagtgagattgtcgaacttccgctgagtgattagctttgcgttcagaagctggcgccaatagttcttcatctttttcacgacttcctctgagggcacgttatctgactttccccggttcttgtctgagcgagtcagcaccttattgtcaattgagtcgtctttgaggaagctctgagggacgatgtggtccacgtcgtagtctgagagcctgttgatatcaagctcttggtccacatacatatcgcgtccattttgaaggtagtacagatagagcttctcattctgaagctgggtgttttccaccgggtgctctttaaggatctgtgacccgagctccttaatcccttcctcaattcttttcatccgctccctgctgttcttctgtcccttctgggtagtctggttttccctggccatctcgatgacaatgttctctggtttatggcgacccatcaccttgaccagctcgtccacgactttcacggtctggagaatacccttcttaatagcgggtgaaccagccagatttgcgatatgctcgtgcagtgagtcaccttgtccagacacttgggccttctggatgtcctccttaaaggtcagggaatcatcatggataagttgcatgaagttgcgattagcgaatccgtcgctcttaaggaaatcaagaatggtctttccgctctgtttgtcccggattccgttgatgagtttgcggctaaggcgtccccatccggtatatcttctccgtttaagttgcttcatcactttatcgtcgaacagatgggcgtaggtcttaagcctctcctcgatcatttcccggtcctcgaacagagtgagagtcaggacaatgtcctcaaggatgtcctcgttctcctcattgtccagaaaatccttgtccttgatgatcttaagcagatcatggtaggtacccaggcttgcgttgaaccgatcttcgaccccgctaatctccacagagtcgaagcattcaattttcttgaaatagtcctccttcagctgcttcacggtcacctttctattggtcttgaacagaaggtccacaattgccttcttttgttcgcctgacaggaatgcgggcttcctcatcccttcggtgacatatttcaccttggtcagctcgttgtacacggtaaagtactcgtagagcagggaatgtttcggcaggaccttctcattggggaggttcttatcgaagttagtcatccgttcgatgaatgactgagcgctggctcccttgtccaccacctcttcgaagttccagggagtgatagtttcttctgactttctggtcatccaagcaaaccggctatttcctctggcgaggggtcccacgtagtaggggatgcggaaagtgagaattttctcaatcttctccctgttgtccttaagaaaggggtagaagtcctcttggcgccgaaggatggcgtgaagctccccaagatggatctggtggggaatgctcccattgtcgaaagtgcgctgcttccgcagcagatcctcccgattcagtttcacgaggagctcttcggtgccgtccatcttctcaaggataggtttgataaacttataaaattcctcttgggaggcacctccgtcgatgtagccggcgtatccattctttgactggtcaaagaagatttccttgtacttctccggaagctgctgccggacgagtgccttaagcagggtcaggtcctggtggtgctcatcataccgcttaatcattgaagctgaaagaggggccttggtgatttcagtgttcacgcggagaatgtcagacagcaggatggcgtcagaaagattcttagcagcaaggaaaaggtcggcgtattggtcaccgatctgggccaggaggttgtcgagatcgtcatcataggtgtccttggacagttgcagcttggcatcctctgccaggtcgaaattagacttgaaattgggagtcagcccaagtgaaagagcaatcaggttcccgaacagtccatttttcttttcacccggcagctgagcgataagattctcaagtctgcggctctttgacagccgggctgacagaatggccttggcatcgactccgcttgcattgatggggttctcttcaaagagctggttgtaagtctgcaccagctggatgaacagtttatccacatcgctattgtcaggattgaggtcgccctcgatcaggaagtggcctctgaacttaatcatgtgggccagggccaggtagataagccggaggtctgccttgtcggttgagtccaccagctttttgcgaagatggtagatggttgggtatttctcgtgataggccacctcgtcgacgatattaccgaagatgggatgccgttcatgcttcttatcctcttccacgaggaatgattcttccagcctatggaagaagctgtcgtcgacctttgccatttcgttggagaatatctcttgaaggtaacaaatccggttcttcctcctggtgtaccgccgtctagcagtccgcttgaggcgggttgcttcagcggtctcccctgaatcaaagaggagagctccgatcagatttttcttgatggaatgccggtcggtgttcccgagcaccttgaacttcttagagggcaccttgtactcgtcagtgatgacggcccacccgacgctattagtcccgatgtccagtccgatagaatacttcttgtccatggtggcgagatctaattcgatccatatcagatcttttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgactagtagactgaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatcgaccggtcgaggagaacttctagtatatctacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagcgccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatagatgctacaggtgttccacttccagatgaggcgctggaagcctccaagaaggctgatgccgttttgttaggtgctgtgggtggtcctaaatggggtacaggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaatttcctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaatttatatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttaacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 54 - DNA, 5204 bp, Artificial Sequence
  pT040 SST22040
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcttagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgattttaggcaaaagttacaatttctggctcgtgtaatatatgtatgctaaagtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagttatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatccatctttgtatacgtctgacgttttagagctagaaatagcaagttaaaataaggctagtccgttatcaacttgaaaaagtggcaccgagtcggtggtgctttttttgttttttatgtcttcgagtcatgtaattagttataagcatgtgagctaagacactgtaattgccaatctaaacgataccacggccgctctagaactagtggatcccccccgctcgagcgggggctgcaggaattcgatatcaagcttatcgataccgtcgacctcgatcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 55 - DNA, 12384 bp, Artificial Sequence
  pML107 FUS150
gacgaaagggcctcgtgatacgcctatttttataggttaatgtcatgataataatggtttcttagatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctgctaaggtagtagacattggttttagagctagaaatagcaagttaaaataaggctagtccgttatcaacttgaaaaagtggcaccgagtcggtggtgctttttttgttttttatgtcttcgagtcatgtaattagttataagcatgtgagctaagacactgtaattgccaatctaaacgataccacggccgctctagagaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcctcgagagtctaggatccggaactacctaccttgcgctttttcttgggagctccctcatcccctccgagctgtgagaggtcaatccgagtttcgtaaagtccggtgatgctctggtggatgagggtggcgtccagcacttctttggtggaggtatagcgtttccggtcgatggtggtgtcgaagtacttgaatgcggcaggggcaccaagattggtgagggtgaacaggtggatgatattctcagcctgctcccgaattggtttgtctctgtgcttattgtatgcgctaaggactttatcgaggttggcatcagcgagaatgacgcgtttagaaaactcggaaatctgctcaatgatctcgtccagataatgcttatgctgctccacaaacagttgcttttgctcgttgtcctcggggctccccttcagcttctcgtaatggcttgcaaggtagaggaaattcacgtacttggaggggagagccagctcattacccttctgaagttcgccagcggaggcgagcatccgcttccgcccattctccagttcgaacaggctatactttgggagcttgatgataaggtccttcttgacttccttgtaacctttggcttcgagaaagtcaattgggttcttctcgaaggagctgcgctccataatggtgatacccagcagttccttcacgctcttgagcttcttagactttcctttttccaccttagccacgaccaggacagaataagccacagttggtgaatcgaatccgccgtacttcttagggtcccaatccttcttccttgcaatgagcttgtcggagttgcgcttggggaggattgattccttagaaaagcctccggtttgcacctcggttttcttcacgatgttcacttgaggcatagaaagcactttgcgcacggtagcgaaatctctgcccttgtcccacacgatttcgccagtctccccattagtctcgatgagcggcctcttccggatctcaccgtttgcaagggtgatttcggtcttgaagaaattcatgatgttgctgtagaagaagtactttgcggtggcctttccgatctcctgctcagatttggcaatcatcttgcgcacgtcgtagaccttgtagtctccgtacacgaactcgctttccagctttgggtacttcttgattagggcggttcccaccacggcattcagatatgcgtcatgagcatgatgatagttattgatctcccgcactttgtagaattgaaagtcctttctgaaatcgctgaccagcttggacttcagggtaatcaccttcacctcgcggataagtttgtcgttttcgtcgtacttggtattcatgcgtgagtcaaggatttgggcgacatgtttagtaatctgccgagtctccacaagttgccgtttaatgaatcctgctttgtccagttcgctcagtccgcccctctcagccttagtgagattgtcgaacttccgctgagtgattagctttgcgttcagaagctggcgccaatagttcttcatctttttcacgacttcctctgagggcacgttatctgactttccccggttcttgtctgagcgagtcagcaccttattgtcaattgagtcgtctttgaggaagctctgagggacgatgtggtccacgtcgtagtctgagagcctgttgatatcaagctcttggtccacatacatatcgcgtccattttgaaggtagtacagatagagcttctcattctgaagctgggtgttttccaccgggtgctctttaaggatctgtgacccgagctccttaatcccttcctcaattcttttcatccgctccctgctgttcttctgtcccttctgggtagtctggttttccctggccatctcgatgacaatgttctctggtttatggcgacccatcaccttgaccagctcgtccacgactttcacggtctggagaatacccttcttaatagcgggtgaaccagccagatttgcgatatgctcgtgcagtgagtcaccttgtccagacacttgggccttctggatgtcctccttaaaggtcagggaatcatcatggataagttgcatgaagttgcgattagcgaatccgtcgctcttaaggaaatcaagaatggtctttccgctctgtttgtcccggattccgttgatgagtttgcggctaaggcgtccccatccggtatatcttctccgtttaagttgcttcatcactttatcgtcgaacagatgggcgtaggtcttaagcctctcctcgatcatttcccggtcctcgaacagagtgagagtcaggacaatgtcctcaaggatgtcctcgttctcctcattgtccagaaaatccttgtccttgatgatcttaagcagatcatggtaggtacccaggcttgcgttgaaccgatcttcgaccccgctaatctccacagagtcgaagcattcaattttcttgaaatagtcctccttcagctgcttcacggtcacctttctattggtcttgaacagaaggtccacaattgccttcttttgttcgcctgacaggaatgcgggcttcctcatcccttcggtgacatatttcaccttggtcagctcgttgtacacggtaaagtactcgtagagcagggaatgtttcggcaggaccttctcattggggaggttcttatcgaagttagtcatccgttcgatgaatgactgagcgctggctcccttgtccaccacctcttcgaagttccagggagtgatagtttcttctgactttctggtcatccaagcaaaccggctatttcctctggcgaggggtcccacgtagtaggggatgcggaaagtgagaattttctcaatcttctccctgttgtccttaagaaaggggtagaagtcctcttggcgccgaaggatggcgtgaagctccccaagatggatctggtggggaatgctcccattgtcgaaagtgcgctgcttccgcagcagatcctcccgattcagtttcacgaggagctcttcggtgccgtccatcttctcaaggataggtttgataaacttataaaattcctcttgggaggcacctccgtcgatgtagccggcgtatccattctttgactggtcaaagaagatttccttgtacttctccggaagctgctgccggacgagtgccttaagcagggtcaggtcctggtggtgctcatcataccgcttaatcattgaagctgaaagaggggccttggtgatttcagtgttcacgcggagaatgtcagacagcaggatggcgtcagaaagattcttagcagcaaggaaaaggtcggcgtattggtcaccgatctgggccaggaggttgtcgagatcgtcatcataggtgtccttggacagttgcagcttggcatcctctgccaggtcgaaattagacttgaaattgggagtcagcccaagtgaaagagcaatcaggttcccgaacagtccatttttcttttcacccggcagctgagcgataagattctcaagtctgcggctctttgacagccgggctgacagaatggccttggcatcgactccgcttgcattgatggggttctcttcaaagagctggttgtaagtctgcaccagctggatgaacagtttatccacatcgctattgtcaggattgaggtcgccctcgatcaggaagtggcctctgaacttaatcatgtgggccagggccaggtagataagccggaggtctgccttgtcggttgagtccaccagctttttgcgaagatggtagatggttgggtatttctcgtgataggccacctcgtcgacgatattaccgaagatgggatgccgttcatgcttcttatcctcttccacgaggaatgattcttccagcctatggaagaagctgtcgtcgacctttgccatttcgttggagaatatctcttgaaggtaacaaatccggttcttcctcctggtgtaccgccgtctagcagtccgcttgaggcgggttgcttcagcggtctcccctgaatcaaagaggagagctccgatcagatttttcttgatggaatgccggtcggtgttcccgagcaccttgaacttcttagagggcaccttgtactcgtcagtgatgacggcccacccgacgctattagtcccgatgtccagtccgatagaatacttcttgtccatggtggcgagatctaattcgatccatatcagatcttttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgactagtagactgaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatcgaccggtcgaggagaacttctagtatatctacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagcgccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatagatgctacaggtgttccacttccagatgaggcgctggaagcctccaagaaggctgatgccgttttgttaggtgctgtgggtggtcctaaatggggtacaggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaatttcctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaatttatatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttaacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 56 - DNA, 5204 bp, Artificial Sequence
  pT040 FUS11524
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcttagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgattttaggcaaaagttacaatttctggctcgtgtaatatatgtatgctaaagtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagttatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcattttcatcagtcgtattctgttttagagctagaaatagcaagttaaaataaggctagtccgttatcaacttgaaaaagtggcaccgagtcggtggtgctttttttgttttttatgtcttcgagtcatgtaattagttataagcatgtgagctaagacactgtaattgccaatctaaacgataccacggccgctctagaactagtggatcccccccgctcgagcgggggctgcaggaattcgatatcaagcttatcgataccgtcgacctcgatcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 57 - DNA, 12384 bp, Artificial Sequence
  pML107 FIG124
gacgaaagggcctcgtgatacgcctatttttataggttaatgtcatgataataatggtttcttagatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctggtcgcaatctcaatgattgttttagagctagaaatagcaagttaaaataaggctagtccgttatcaacttgaaaaagtggcaccgagtcggtggtgctttttttgttttttatgtcttcgagtcatgtaattagttataagcatgtgagctaagacactgtaattgccaatctaaacgataccacggccgctctagagaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcctcgagagtctaggatccggaactacctaccttgcgctttttcttgggagctccctcatcccctccgagctgtgagaggtcaatccgagtttcgtaaagtccggtgatgctctggtggatgagggtggcgtccagcacttctttggtggaggtatagcgtttccggtcgatggtggtgtcgaagtacttgaatgcggcaggggcaccaagattggtgagggtgaacaggtggatgatattctcagcctgctcccgaattggtttgtctctgtgcttattgtatgcgctaaggactttatcgaggttggcatcagcgagaatgacgcgtttagaaaactcggaaatctgctcaatgatctcgtccagataatgcttatgctgctccacaaacagttgcttttgctcgttgtcctcggggctccccttcagcttctcgtaatggcttgcaaggtagaggaaattcacgtacttggaggggagagccagctcattacccttctgaagttcgccagcggaggcgagcatccgcttccgcccattctccagttcgaacaggctatactttgggagcttgatgataaggtccttcttgacttccttgtaacctttggcttcgagaaagtcaattgggttcttctcgaaggagctgcgctccataatggtgatacccagcagttccttcacgctcttgagcttcttagactttcctttttccaccttagccacgaccaggacagaataagccacagttggtgaatcgaatccgccgtacttcttagggtcccaatccttcttccttgcaatgagcttgtcggagttgcgcttggggaggattgattccttagaaaagcctccggtttgcacctcggttttcttcacgatgttcacttgaggcatagaaagcactttgcgcacggtagcgaaatctctgcccttgtcccacacgatttcgccagtctccccattagtctcgatgagcggcctcttccggatctcaccgtttgcaagggtgatttcggtcttgaagaaattcatgatgttgctgtagaagaagtactttgcggtggcctttccgatctcctgctcagatttggcaatcatcttgcgcacgtcgtagaccttgtagtctccgtacacgaactcgctttccagctttgggtacttcttgattagggcggttcccaccacggcattcagatatgcgtcatgagcatgatgatagttattgatctcccgcactttgtagaattgaaagtcctttctgaaatcgctgaccagcttggacttcagggtaatcaccttcacctcgcggataagtttgtcgttttcgtcgtacttggtattcatgcgtgagtcaaggatttgggcgacatgtttagtaatctgccgagtctccacaagttgccgtttaatgaatcctgctttgtccagttcgctcagtccgcccctctcagccttagtgagattgtcgaacttccgctgagtgattagctttgcgttcagaagctggcgccaatagttcttcatctttttcacgacttcctctgagggcacgttatctgactttccccggttcttgtctgagcgagtcagcaccttattgtcaattgagtcgtctttgaggaagctctgagggacgatgtggtccacgtcgtagtctgagagcctgttgatatcaagctcttggtccacatacatatcgcgtccattttgaaggtagtacagatagagcttctcattctgaagctgggtgttttccaccgggtgctctttaaggatctgtgacccgagctccttaatcccttcctcaattcttttcatccgctccctgctgttcttctgtcccttctgggtagtctggttttccctggccatctcgatgacaatgttctctggtttatggcgacccatcaccttgaccagctcgtccacgactttcacggtctggagaatacccttcttaatagcgggtgaaccagccagatttgcgatatgctcgtgcagtgagtcaccttgtccagacacttgggccttctggatgtcctccttaaaggtcagggaatcatcatggataagttgcatgaagttgcgattagcgaatccgtcgctcttaaggaaatcaagaatggtctttccgctctgtttgtcccggattccgttgatgagtttgcggctaaggcgtccccatccggtatatcttctccgtttaagttgcttcatcactttatcgtcgaacagatgggcgtaggtcttaagcctctcctcgatcatttcccggtcctcgaacagagtgagagtcaggacaatgtcctcaaggatgtcctcgttctcctcattgtccagaaaatccttgtccttgatgatcttaagcagatcatggtaggtacccaggcttgcgttgaaccgatcttcgaccccgctaatctccacagagtcgaagcattcaattttcttgaaatagtcctccttcagctgcttcacggtcacctttctattggtcttgaacagaaggtccacaattgccttcttttgttcgcctgacaggaatgcgggcttcctcatcccttcggtgacatatttcaccttggtcagctcgttgtacacggtaaagtactcgtagagcagggaatgtttcggcaggaccttctcattggggaggttcttatcgaagttagtcatccgttcgatgaatgactgagcgctggctcccttgtccaccacctcttcgaagttccagggagtgatagtttcttctgactttctggtcatccaagcaaaccggctatttcctctggcgaggggtcccacgtagtaggggatgcggaaagtgagaattttctcaatcttctccctgttgtccttaagaaaggggtagaagtcctcttggcgccgaaggatggcgtgaagctccccaagatggatctggtggggaatgctcccattgtcgaaagtgcgctgcttccgcagcagatcctcccgattcagtttcacgaggagctcttcggtgccgtccatcttctcaaggataggtttgataaacttataaaattcctcttgggaggcacctccgtcgatgtagccggcgtatccattctttgactggtcaaagaagatttccttgtacttctccggaagctgctgccggacgagtgccttaagcagggtcaggtcctggtggtgctcatcataccgcttaatcattgaagctgaaagaggggccttggtgatttcagtgttcacgcggagaatgtcagacagcaggatggcgtcagaaagattcttagcagcaaggaaaaggtcggcgtattggtcaccgatctgggccaggaggttgtcgagatcgtcatcataggtgtccttggacagttgcagcttggcatcctctgccaggtcgaaattagacttgaaattgggagtcagcccaagtgaaagagcaatcaggttcccgaacagtccatttttcttttcacccggcagctgagcgataagattctcaagtctgcggctctttgacagccgggctgacagaatggccttggcatcgactccgcttgcattgatggggttctcttcaaagagctggttgtaagtctgcaccagctggatgaacagtttatccacatcgctattgtcaggattgaggtcgccctcgatcaggaagtggcctctgaacttaatcatgtgggccagggccaggtagataagccggaggtctgccttgtcggttgagtccaccagctttttgcgaagatggtagatggttgggtatttctcgtgataggccacctcgtcgacgatattaccgaagatgggatgccgttcatgcttcttatcctcttccacgaggaatgattcttccagcctatggaagaagctgtcgtcgacctttgccatttcgttggagaatatctcttgaaggtaacaaatccggttcttcctcctggtgtaccgccgtctagcagtccgcttgaggcgggttgcttcagcggtctcccctgaatcaaagaggagagctccgatcagatttttcttgatggaatgccggtcggtgttcccgagcaccttgaacttcttagagggcaccttgtactcgtcagtgatgacggcccacccgacgctattagtcccgatgtccagtccgatagaatacttcttgtccatggtggcgagatctaattcgatccatatcagatcttttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgactagtagactgaattcgatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatcgaccggtcgaggagaacttctagtatatctacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagcgccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatagatgctacaggtgttccacttccagatgaggcgctggaagcctccaagaaggctgatgccgttttgttaggtgctgtgggtggtcctaaatggggtacaggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaatttcctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaatttatatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttaacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 58 - DNA, 5204 bp, Artificial Sequence
  pT040 FIG1887
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcttagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgattttaggcaaaagttacaatttctggctcgtgtaatatatgtatgctaaagtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagttatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttgaggataaaactaaacttgttttagagctagaaatagcaagttaaaataaggctagtccgttatcaacttgaaaaagtggcaccgagtcggtggtgctttttttgttttttatgtcttcgagtcatgtaattagttataagcatgtgagctaagacactgtaattgccaatctaaacgataccacggccgctctagaactagtggatcccccccgctcgagcgggggctgcaggaattcgatatcaagcttatcgataccgtcgacctcgatcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 59 - DNA, 11269 bp, Artificial Sequence
  pML104 X2
aacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctccaccgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcctctcgaagtggtcacgtgcgttttagagctagaaatagcaagttaaaataaggctagtccgttatcaacttgaaaaagtggcaccgagtcggtggtgctttttttgttttttatgtcttcgagtcatgtaattagttataagcatgtgagctaagacactgtaattgccaatctaaacgataccacggccgctctagagaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcctcgagagtctaggatccggaactacctaccttgcgctttttcttgggagctccctcatcccctccgagctgtgagaggtcaatccgagtttcgtaaagtccggtgatgctctggtggatgagggtggcgtccagcacttctttggtggaggtatagcgtttccggtcgatggtggtgtcgaagtacttgaatgcggcaggggcaccaagattggtgagggtgaacaggtggatgatattctcagcctgctcccgaattggtttgtctctgtgcttattgtatgcgctaaggactttatcgaggttggcatcagcgagaatgacgcgtttagaaaactcggaaatctgctcaatgatctcgtccagataatgcttatgctgctccacaaacagttgcttttgctcgttgtcctcggggctccccttcagcttctcgtaatggcttgcaaggtagaggaaattcacgtacttggaggggagagccagctcattacccttctgaagttcgccagcggaggcgagcatccgcttccgcccattctccagttcgaacaggctatactttgggagcttgatgataaggtccttcttgacttccttgtaacctttggcttcgagaaagtcaattgggttcttctcgaaggagctgcgctccataatggtgatacccagcagttccttcacgctcttgagcttcttagactttcctttttccaccttagccacgaccaggacagaataagccacagttggtgaatcgaatccgccgtacttcttagggtcccaatccttcttccttgcaatgagcttgtcggagttgcgcttggggaggattgattccttagaaaagcctccggtttgcacctcggttttcttcacgatgttcacttgaggcatagaaagcactttgcgcacggtagcgaaatctctgcccttgtcccacacgatttcgccagtctccccattagtctcgatgagcggcctcttccggatctcaccgtttgcaagggtgatttcggtcttgaagaaattcatgatgttgctgtagaagaagtactttgcggtggcctttccgatctcctgctcagatttggcaatcatcttgcgcacgtcgtagaccttgtagtctccgtacacgaactcgctttccagctttgggtacttcttgattagggcggttcccaccacggcattcagatatgcgtcatgagcatgatgatagttattgatctcccgcactttgtagaattgaaagtcctttctgaaatcgctgaccagcttggacttcagggtaatcaccttcacctcgcggataagtttgtcgttttcgtcgtacttggtattcatgcgtgagtcaaggatttgggcgacatgtttagtaatctgccgagtctccacaagttgccgtttaatgaatcctgctttgtccagttcgctcagtccgcccctctcagccttagtgagattgtcgaacttccgctgagtgattagctttgcgttcagaagctggcgccaatagttcttcatctttttcacgacttcctctgagggcacgttatctgactttccccggttcttgtctgagcgagtcagcaccttattgtcaattgagtcgtctttgaggaagctctgagggacgatgtggtccacgtcgtagtctgagagcctgttgatatcaagctcttggtccacatacatatcgcgtccattttgaaggtagtacagatagagcttctcattctgaagctgggtgttttccaccgggtgctctttaaggatctgtgacccgagctccttaatcccttcctcaattcttttcatccgctccctgctgttcttctgtcccttctgggtagtctggttttccctggccatctcgatgacaatgttctctggtttatggcgacccatcaccttgaccagctcgtccacgactttcacggtctggagaatacccttcttaatagcgggtgaaccagccagatttgcgatatgctcgtgcagtgagtcaccttgtccagacacttgggccttctggatgtcctccttaaaggtcagggaatcatcatggataagttgcatgaagttgcgattagcgaatccgtcgctcttaaggaaatcaagaatggtctttccgctctgtttgtcccggattccgttgatgagtttgcggctaaggcgtccccatccggtatatcttctccgtttaagttgcttcatcactttatcgtcgaacagatgggcgtaggtcttaagcctctcctcgatcatttcccggtcctcgaacagagtgagagtcaggacaatgtcctcaaggatgtcctcgttctcctcattgtccagaaaatccttgtccttgatgatcttaagcagatcatggtaggtacccaggcttgcgttgaaccgatcttcgaccccgctaatctccacagagtcgaagcattcaattttcttgaaatagtcctccttcagctgcttcacggtcacctttctattggtcttgaacagaaggtccacaattgccttcttttgttcgcctgacaggaatgcgggcttcctcatcccttcggtgacatatttcaccttggtcagctcgttgtacacggtaaagtactcgtagagcagggaatgtttcggcaggaccttctcattggggaggttcttatcgaagttagtcatccgttcgatgaatgactgagcgctggctcccttgtccaccacctcttcgaagttccagggagtgatagtttcttctgactttctggtcatccaagcaaaccggctatttcctctggcgaggggtcccacgtagtaggggatgcggaaagtgagaattttctcaatcttctccctgttgtccttaagaaaggggtagaagtcctcttggcgccgaaggatggcgtgaagctccccaagatggatctggtggggaatgctcccattgtcgaaagtgcgctgcttccgcagcagatcctcccgattcagtttcacgaggagctcttcggtgccgtccatcttctcaaggataggtttgataaacttataaaattcctcttgggaggcacctccgtcgatgtagccggcgtatccattctttgactggtcaaagaagatttccttgtacttctccggaagctgctgccggacgagtgccttaagcagggtcaggtcctggtggtgctcatcataccgcttaatcattgaagctgaaagaggggccttggtgatttcagtgttcacgcggagaatgtcagacagcaggatggcgtcagaaagattcttagcagcaaggaaaaggtcggcgtattggtcaccgatctgggccaggaggttgtcgagatcgtcatcataggtgtccttggacagttgcagcttggcatcctctgccaggtcgaaattagacttgaaattgggagtcagcccaagtgaaagagcaatcaggttcccgaacagtccatttttcttttcacccggcagctgagcgataagattctcaagtctgcggctctttgacagccgggctgacagaatggccttggcatcgactccgcttgcattgatggggttctcttcaaagagctggttgtaagtctgcaccagctggatgaacagtttatccacatcgctattgtcaggattgaggtcgccctcgatcaggaagtggcctctgaacttaatcatgtgggccagggccaggtagataagccggaggtctgccttgtcggttgagtccaccagctttttgcgaagatggtagatggttgggtatttctcgtgataggccacctcgtcgacgatattaccgaagatgggatgccgttcatgcttcttatcctcttccacgaggaatgattcttccagcctatggaagaagctgtcgtcgacctttgccatttcgttggagaatatctcttgaaggtaacaaatccggttcttcctcctggtgtaccgccgtctagcagtccgcttgaggcgggttgcttcagcggtctcccctgaatcaaagaggagagctccgatcagatttttcttgatggaatgccggtcggtgttcccgagcaccttgaacttcttagagggcaccttgtactcgtcagtgatgacggcccacccgacgctattagtcccgatgtccagtccgatagaatacttcttgtccatggtggcgagatctaattcgatccatatcagatcttttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgactagtagactgaattcgatatcaagcttatcgataccgtcgacctcgagggggggcccggtacccaattcgccctatagtgagtcgtattacgcgcgctcactggccgtcgttttac

SEQ ID NO: 60 - DNA, 11269 bp, Artificial Sequence
  pML104 X-2 UnTS
aacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctccaccgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttgcgtaagtggcccctagcgttttagagctagaaatagcaagttaaaataaggctagtccgttatcaacttgaaaaagtggcaccgagtcggtggtgctttttttgttttttatgtcttcgagtcatgtaattagttataagcatgtgagctaagacactgtaattgccaatctaaacgataccacggccgctctagagaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcctcgagagtctaggatccggaactacctaccttgcgctttttcttgggagctccctcatcccctccgagctgtgagaggtcaatccgagtttcgtaaagtccggtgatgctctggtggatgagggtggcgtccagcacttctttggtggaggtatagcgtttccggtcgatggtggtgtcgaagtacttgaatgcggcaggggcaccaagattggtgagggtgaacaggtggatgatattctcagcctgctcccgaattggtttgtctctgtgcttattgtatgcgctaaggactttatcgaggttggcatcagcgagaatgacgcgtttagaaaactcggaaatctgctcaatgatctcgtccagataatgcttatgctgctccacaaacagttgcttttgctcgttgtcctcggggctccccttcagcttctcgtaatggcttgcaaggtagaggaaattcacgtacttggaggggagagccagctcattacccttctgaagttcgccagcggaggcgagcatccgcttccgcccattctccagttcgaacaggctatactttgggagcttgatgataaggtccttcttgacttccttgtaacctttggcttcgagaaagtcaattgggttcttctcgaaggagctgcgctccataatggtgatacccagcagttccttcacgctcttgagcttcttagactttcctttttccaccttagccacgaccaggacagaataagccacagttggtgaatcgaatccgccgtacttcttagggtcccaatccttcttccttgcaatgagcttgtcggagttgcgcttggggaggattgattccttagaaaagcctccggtttgcacctcggttttcttcacgatgttcacttgaggcatagaaagcactttgcgcacggtagcgaaatctctgcccttgtcccacacgatttcgccagtctccccattagtctcgatgagcggcctcttccggatctcaccgtttgcaagggtgatttcggtcttgaagaaattcatgatgttgctgtagaagaagtactttgcggtggcctttccgatctcctgctcagatttggcaatcatcttgcgcacgtcgtagaccttgtagtctccgtacacgaactcgctttccagctttgggtacttcttgattagggcggttcccaccacggcattcagatatgcgtcatgagcatgatgatagttattgatctcccgcactttgtagaattgaaagtcctttctgaaatcgctgaccagcttggacttcagggtaatcaccttcacctcgcggataagtttgtcgttttcgtcgtacttggtattcatgcgtgagtcaaggatttgggcgacatgtttagtaatctgccgagtctccacaagttgccgtttaatgaatcctgctttgtccagttcgctcagtccgcccctctcagccttagtgagattgtcgaacttccgctgagtgattagctttgcgttcagaagctggcgccaatagttcttcatctttttcacgacttcctctgagggcacgttatctgactttccccggttcttgtctgagcgagtcagcaccttattgtcaattgagtcgtctttgaggaagctctgagggacgatgtggtccacgtcgtagtctgagagcctgttgatatcaagctcttggtccacatacatatcgcgtccattttgaaggtagtacagatagagcttctcattctgaagctgggtgttttccaccgggtgctctttaaggatctgtgacccgagctccttaatcccttcctcaattcttttcatccgctccctgctgttcttctgtcccttctgggtagtctggttttccctggccatctcgatgacaatgttctctggtttatggcgacccatcaccttgaccagctcgtccacgactttcacggtctggagaatacccttcttaatagcgggtgaaccagccagatttgcgatatgctcgtgcagtgagtcaccttgtccagacacttgggccttctggatgtcctccttaaaggtcagggaatcatcatggataagttgcatgaagttgcgattagcgaatccgtcgctcttaaggaaatcaagaatggtctttccgctctgtttgtcccggattccgttgatgagtttgcggctaaggcgtccccatccggtatatcttctccgtttaagttgcttcatcactttatcgtcgaacagatgggcgtaggtcttaagcctctcctcgatcatttcccggtcctcgaacagagtgagagtcaggacaatgtcctcaaggatgtcctcgttctcctcattgtccagaaaatccttgtccttgatgatcttaagcagatcatggtaggtacccaggcttgcgttgaaccgatcttcgaccccgctaatctccacagagtcgaagcattcaattttcttgaaatagtcctccttcagctgcttcacggtcacctttctattggtcttgaacagaaggtccacaattgccttcttttgttcgcctgacaggaatgcgggcttcctcatcccttcggtgacatatttcaccttggtcagctcgttgtacacggtaaagtactcgtagagcagggaatgtttcggcaggaccttctcattggggaggttcttatcgaagttagtcatccgttcgatgaatgactgagcgctggctcccttgtccaccacctcttcgaagttccagggagtgatagtttcttctgactttctggtcatccaagcaaaccggctatttcctctggcgaggggtcccacgtagtaggggatgcggaaagtgagaattttctcaatcttctccctgttgtccttaagaaaggggtagaagtcctcttggcgccgaaggatggcgtgaagctccccaagatggatctggtggggaatgctcccattgtcgaaagtgcgctgcttccgcagcagatcctcccgattcagtttcacgaggagctcttcggtgccgtccatcttctcaaggataggtttgataaacttataaaattcctcttgggaggcacctccgtcgatgtagccggcgtatccattctttgactggtcaaagaagatttccttgtacttctccggaagctgctgccggacgagtgccttaagcagggtcaggtcctggtggtgctcatcataccgcttaatcattgaagctgaaagaggggccttggtgatttcagtgttcacgcggagaatgtcagacagcaggatggcgtcagaaagattcttagcagcaaggaaaaggtcggcgtattggtcaccgatctgggccaggaggttgtcgagatcgtcatcataggtgtccttggacagttgcagcttggcatcctctgccaggtcgaaattagacttgaaattgggagtcagcccaagtgaaagagcaatcaggttcccgaacagtccatttttcttttcacccggcagctgagcgataagattctcaagtctgcggctctttgacagccgggctgacagaatggccttggcatcgactccgcttgcattgatggggttctcttcaaagagctggttgtaagtctgcaccagctggatgaacagtttatccacatcgctattgtcaggattgaggtcgccctcgatcaggaagtggcctctgaacttaatcatgtgggccagggccaggtagataagccggaggtctgccttgtcggttgagtccaccagctttttgcgaagatggtagatggttgggtatttctcgtgataggccacctcgtcgacgatattaccgaagatgggatgccgttcatgcttcttatcctcttccacgaggaatgattcttccagcctatggaagaagctgtcgtcgacctttgccatttcgttggagaatatctcttgaaggtaacaaatccggttcttcctcctggtgtaccgccgtctagcagtccgcttgaggcgggttgcttcagcggtctcccctgaatcaaagaggagagctccgatcagatttttcttgatggaatgccggtcggtgttcccgagcaccttgaacttcttagagggcaccttgtactcgtcagtgatgacggcccacccgacgctattagtcccgatgtccagtccgatagaatacttcttgtccatggtggcgagatctaattcgatccatatcagatcttttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgactagtagactgaattcgatatcaagcttatcgataccgtcgacctcgagggggggcccggtacccaattcgccctatagtgagtcgtattacgcgcgctcactggccgtcgttttac

SEQ ID NO: 61 - DNA, 4110 bp, Artificial Sequence
  pMARQ-X2LP-CYC1b
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggtacctacacgtacttagtcgctgaagctcttctatggtaatgagacgaaaaagctcgaaatgaatggatatattctttttgctactggcaactgttgaatatttaatgttaaaacaaactaactgaggtatattcgtatctgtatgtacacatatactatatacaggaaaagataagcaagagagaggatatcaactacgagagcgatcgattatatatcaaaagctgtccgctttgccacccataatcggcgcttagtttcggagttcaatcataattctaccaccttacactcaacttactctttaactcctatagtataatatcgccactgaccccatattaaaaaaatttttttgctcgatcttctatcctctttaggttaattgtcgctgttattgtctagattttttctcggagatggcgcatctatttgccgtcaaaagatcctctcataccatattaagtaaattgcctccatttctttttcctcgggcagagaaactcgcaggcaacttgctctcgaagtggtcacgcacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaattgcgtaagtggcccctagcggggcagtggtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttggcataatcggccctcacagagggatcccgttacccatctatgctgaagatttatcatactattcctccgctcgtttcttttttcagtgaggtgtgtcgtgaaagaaaacccacaattaaactttcaacaaccgggcgactaggaagagagtagtgggcgcggatgacgaaggctaaggtcacttcttcgtttcctttattggggtttccgtgtagccttcccctgaatagtgtgggacgttttatgagaagccgtaagaaataggcaaattgagttatgacaagtagacatgatgccgcagccttgcctgactttacgtctccttcatgaataagtttttctatcgagttcttttccttttttcgccttaattagctcaattaagcctgtcctcactacttttctttttcttatcggctttgtgccacacctaaccttcgaatgctgttttattccgttcttacatgggatggtaatgccttggcgagaatttcgattttggtagaagagccgtcaatcgagttcgtacctagagctcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 62 - DNA, 4900 bp, Artificial Sequence
  pSJ1458 (pFA6-link-mTurquoise2-CaURA3MX)
cgaattcatcgatgatatcagatccactagtggcctatgcggccgcggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacgcggccgccagctgaagcttcgtacgctgcaggtcgacggatcggtgacggtgctggtttaattaacatgtctaaaggtgaagaattgtttactggtgttgttccaattttggttgaattggatggtgatgttaatggtcataaattttctgtttctggtgaaggtgaaggtgatgctacttatggtaaattgactttgaaatttatttgtactactggtaaattgccagttccatggccaactttggttactactttgtcttggggtgttcaatgttttgctagatatccagatcatatgaaacaacatgatttttttaaatctgctatgccagaaggttatgttcaagaaagaactattttttttaaagatgatggtaattataaaactagagctgaagttaaatttgaaggtgatactttggttaatagaattgaattgaaaggtattgattttaaagaagatggtaatattttgggtcataaattggaatataattatttttctgataatgtttatattactgctgataaacaaaaaaatggtattaaagctaattttaaaattagacataatattgaagatggtggtgttcaattggctgatcattatcaacaaaatactccaattggtgatggtccagttttgttgccagataatcattatttgtctactcaatctgctttgtctaaagatccaaatgaaaaaagagatcatatggttttgttggaatttgttactgctgctggtattactttgggtatggatgaattgtataaataaggcgcgccacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagtatgaggtcgctcttattgaccacacctctaccggcagatccgctagggataacagggtaatatagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgacagtcaacactaagacctatagtgagagagcagaaactcatgcctcaccagtagcacagcgattatttcgattaatggaactgaagaaaaccaatttatgtgcatcaattgacgttgataccactaaggaattccttgaattaattgataaattaggtccttatgtatgcttaatcaagactcatattgatataatcaatgatttttcctatgaatccactattgaaccattattagaactttcacgtaaacatcaatttatgatttttgaagatagaaaatttgctgatattggtaataccgtaaagaaacaatatattggtggagtttataaaattagtagttgggcagatattaccaatgctcatggtgtcactgggaatggagtggttgaaggattaaaacagggagctaaagaaaccaccaccaaccaagagccaagagggttattgatgttagctgaattatcatcagtgggatcattagcatatggagaatattctcaaaaaactgttgaaattgctaaatccgataaggaatttgttattggatttattgcccaacgtgatatgggtggccaagaagaaggatttgattggcttattatgacacctggagttggattagatgataaaggtgatggattaggacaacaatatagaactgttgatgaagttgttagcactggaactgatattatcattgttggtagaggattgtttggtaaaggaagagatccagatattgaaggtaaaaggtatagaaatgctggttggaatgcttatttgaaaaagactggccaattataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagct

SEQ ID NO: 63 - DNA, 3627 bp, Artificial Sequence
  pNCS-mRuby3
gatctcgatcccgcgaaattaatacgactcactatagggagaccacaacggtttccctctagaatcaccgagtttattcttgacacctgatgcgatgaatgatataataggaaagtactgttttgtttaactttaagaaggagatatacatatgcggggttctcatcatcatcatcatcatggtatggctagcatgactggtggacagcaaatgggtcgggatctgtacgacgatgacgataaggatccgatggtgtctaagggcgaagagctgatcaaggaaaatatgcgtatgaaggtggtcatggaaggttcggtcaacggccaccaattcaaatgcacaggtgaaggagaaggcagaccgtacgagggaactcaaaccatgaggatcaaagtcatcgagggaggacccctgccatttgcctttgacattcttgccacgtcgttcatgtatggcagccgtacttttatcaagtacccggccgacatccctgatttctttaaacagtcctttcctgagggttttacttgggaaagagttacgagatacgaagatggtggagtcgtcaccgtcacgcaggacaccagccttgaggatggcgagctcgtctacaacgtcaaggtcagaggggtaaactttccctccaatggtcccgtgatgcagaagaagaccaagggttgggagcctaatacagagatgatgtatccagcagatggtggtctgagaggatacactgacatcgcactgaaagttgatggtggtggccatctgcactgcaacttcgtgacaacttacaggtcaaaaaagaccgtcgggaacatcaagatgcccggtgtccatgccgttgatcaccgcctggaaaggatcgaggagagtgacaatgaaacctacgtagtgcaacgcgaagtggcagttgccaaatacagcaaccttggtggtggcatggacgagctgtacaagtaatgaattcgaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 64 - DNA, 37 bp, Artificial Sequence
  gRNA FAR110 1
gatcttttcaaacgaaactcttgtgttttagagctag

SEQ ID NO: 65 - DNA, 33 bp, Artificial Sequence
  gRNA FAR110 2
ctagctctaaaacacaagagtttcgtttgaaaa

SEQ ID NO: 66 - DNA, 37 bp, Artificial Sequence
  gRNA FAR12442 1
gatcgaattctttgctgctttaccgttttagagctag

SEQ ID NO: 67 - DNA, 33 bp, Artificial Sequence
  gRNA FAR12442 2
ctagctctaaaacggtaaagcagcaaagaattc

SEQ ID NO: 68 - DNA, 19 bp, Artificial Sequence
  FAR1 Seq F
gacattgcacttgcatcac

SEQ ID NO: 69 - DNA, 19 bp, Artificial Sequence
  FAR1 Seq R
cacgtgacctgaaagtcac

SEQ ID NO: 70 - DNA, 37 bp, Artificial Sequence
  gRNA SST2-19 1
gatcttaaagatagagttgtaagagttttagagctag

SEQ ID NO: 71 - DNA, 33 bp, Artificial Sequence
  gRNA SST2-19 2
ctagctctaaaactcttacaactctatctttaa

SEQ ID NO: 72 - DNA, 37 bp, Artificial Sequence
  gRNA SST22040 1
gatccatctttgtatacgtctgacgttttagagctag

SEQ ID NO: 73 - DNA, 33 bp, Artificial Sequence
  gRNA SST22040 2
ctagctctaaaacgtcagacgtatacaaagatg

SEQ ID NO: 74 - DNA, 22 bp, Artificial Sequence
  SST2 Seq F
ctgtctttacttctcatcgtcc

SEQ ID NO: 75 - DNA, 31 bp, Artificial Sequence
  SST2 Seq R
ccgcttaatttatacgtaatagtatttacag

SEQ ID NO: 76 - DNA, 37 bp, Artificial Sequence
  gRNA STE237
gatctatgatccaacgtataatccgttttagagctag

SEQ ID NO: 77 - DNA, 33 bp, Artificial Sequence
  gRNA STE237 2
ctagctctaaaacggattatacgttggatcata

SEQ ID NO: 78 - DNA, 37 bp, Artificial Sequence
  gRNA STE21259 1
gatcatcttcagtccagaactttcgttttagagctag

SEQ ID NO: 79 - DNA, 33 bp, Artificial Sequence
  gRNA STE21259 2
ctagctctaaaacgaaagttctggactgaagat

SEQ ID NO: 80 - DNA, 22 bp, Artificial Sequence
  Ste2 Seq F
ttatccaatgcctgccaaaatg

SEQ ID NO: 81 - DNA, 20 bp, Artificial Sequence
  Ste2 Seq R
caaaatgccaaacagcgtac

SEQ ID NO: 82 - DNA, 37 bp, Artificial Sequence
  gRNA FIG124 1
gatctggtcgcaatctcaatgattgttttagagctag

SEQ ID NO: 83 - DNA, 33 bp, Artificial Sequence
  gRNA FIG124 2
ctagctctaaaacaatcattgagattgcgacca

SEQ ID NO: 84 - DNA, 37 bp, Artificial Sequence
  gRNA FIG1887 1
gatcttgaggataaaactaaacttgttttagagctag

SEQ ID NO: 85 - DNA, 33 bp, Artificial Sequence
  gRNA FIG1887 2
ctagctctaaaacaagtttagttttatcctcaa

SEQ ID NO: 86 - DNA, 23 bp, Artificial Sequence
  FIG1 Seq F
gaaaacaatatatagtgctgttg

SEQ ID NO: 87 - DNA, 22 bp, Artificial Sequence
  FIG1 Seq R
gttgttacttaattatcgaacg

SEQ ID NO: 88 - DNA, 37 bp, Artificial Sequence
  gRNA X2 1
gatcctctcgaagtggtcacgtgcgttttagagctag

SEQ ID NO: 89 - DNA, 33 bp, Artificial Sequence
  gRNA X2 2
ctagctctaaaacgcacgtgaccacttcgagag

SEQ ID NO: 90 - DNA, 37 bp, Artificial Sequence
  gRNA X2 UnTS 1
gatcttgcgtaagtggcccctagcgttttagagctag

SEQ ID NO: 91 - DNA, 33 bp, Artificial Sequence
  gRNA X2 UnTS 2
ctagctctaaaacgctaggggccacttacgcaa

SEQ ID NO: 92 - DNA, 21 bp, Artificial Sequence
  CRISPR X2 LP 60 F
tccatttctttttcctcgggc

SEQ ID NO: 93 - DNA, 28 bp, Artificial Sequence
  CRISPR X2 LP 60 R
agtatgataaatcttcagcatagatggg

SEQ ID NO: 94 - DNA, 22 bp, Artificial Sequence
  CRISPR X2 LP 110 F
cgcatctatttgccgtcaaaag

SEQ ID NO: 95 - DNA, 21 bp, Artificial Sequence
  CRISPR X2 LP 110 R
ggttttctttcacgacacacc

SEQ ID NO: 96 - DNA, 22 bp, Artificial Sequence
  GPA1 Cterm Seq F
acagaaaccgaatttaacatcg

SEQ ID NO: 97 - DNA, 23 bp, Artificial Sequence
  GPA1 Cterm Seq R
tcttgattctttgtcacctcaac

SEQ ID NO: 98 - DNA, 90 bp, Artificial Sequence
  FIG1::mTq2 UltF
gcttgtctttggtagaagaaattatagtaaacaaacaaacaaacaaacaaaaaaaaaaaaaaaaaaatgtctaaaggtgaagaattgttt

SEQ ID NO: 99 - DNA, 90 bp, Artificial Sequence
  FIG1::mTq2 UltR
ttacttttaaaaattgacattctgaaatatccgctcaaaacttatatgtttatttgaggataaaattatttatacaattcatccataccc

SEQ ID NO: 100 - DNA, 90 bp, Artificial Sequence
  FUS1::mTq2 UltF
tacgacatcctttatcttttttcctttaagagcaggatataagccatcaagtttctgaaaatcaaaatgtctaaaggtgaagaattgttt

SEQ ID NO: 101 - DNA, 90 bp, Artificial Sequence
  FUS1::mTq2 UltR
aaggaacagtatataagtacagaattataggtatagattaaatgcgaacgtcaatattattttcattatttatacaattcatccataccc

SEQ ID NO: 102 - DNA, 60 bp, Artificial Sequence
  mTq2 TEF F
aagcatagcaatctaatctaagttttaattacaaatgtctaaaggtgaagaattgtttac

SEQ ID NO: 103 - DNA, 55 bp, Artificial Sequence
  mTq2 CYC1b R
gtgaatgtaagcgtgacataactaatttatttatacaattcatccatacccaaag

SEQ ID NO: 104 - DNA, 60 bp, Artificial Sequence
  mRuby3 TEF F
attagaaagaaagcatagcaatctaatctaagttttaattacaaatggtgtctaagggcg

SEQ ID NO: 105 - DNA, 60 bp, Artificial Sequence
  mRuby3 CYC1b R
atgtgggaggagggcgtgaatgtaagcgtgacataactaatttacttgtacagctcgtcc

SEQ ID NO: 106 - DNA, 60 bp, Artificial Sequence
  TEF 60 UF
tttttacttcttgctcattagaaagaaagcatagcaatctaatctaagttttaattacaa

SEQ ID NO: 107 - DNA, 60 bp, Artificial Sequence
  CYC1b 60 UR
tccttttcggttagagcggatgtgggaggagggcgtgaatgtaagcgtgacataactaat

SEQ ID NO: 108 - DNA, 55 bp, Artificial Sequence
  ADORA1 TEF F
gaaagcatagcaatctaatctaagttttaattacaaatgcctcccagtatatccg

SEQ ID NO: 109 - DNA, 45 bp, Artificial Sequence
  ADORA1 CYC1b R
gtgaatgtaagcgtgacataactaatttagtcgtcaggccgttcc

SEQ ID NO: 110 - DNA, 57 bp, Artificial Sequence
  ADORA2A TEF F
gaaagcatagcaatctaatctaagttttaattacaaatgcctatcatgggttctagc

SEQ ID NO: 111 - DNA, 47 bp, Artificial Sequence
  ADORA2A CYC1b R
gtgaatgtaagcgtgacataactaatttaagaaaccccagcaccatc

SEQ ID NO: 112 - DNA, 54 bp, Artificial Sequence
  ADORA2B TEF F
gaaagcatagcaatctaatctaagttttaattacaaatgttgcttgagacccag

SEQ ID NO: 113 - DNA, 45 bp, Artificial Sequence
  ADORA2B CYC1b R
gtgaatgtaagcgtgacataactaatttacagccccactcccaag

SEQ ID NO: 114 - DNA, 52 bp, Artificial Sequence
  ADRA2A TEF F
gaaagcatagcaatctaatctaagttttaattacaaatgttccggcaggagc

SEQ ID NO: 115 - DNA, 49 bp, Artificial Sequence
  ADRA2A CYC1b R
gtgaatgtaagcgtgacataactaatttaaacgattcgtttcctatcgc

SEQ ID NO: 116 - DNA, 59 bp, Artificial Sequence
  ADRA2B TEF F
gaaagcatagcaatctaatctaagttttaattacaaatggatcatcaggatccctattc

SEQ ID NO: 117 - DNA, 46 bp, Artificial Sequence
  ADRA2B CYC1b R
gtgaatgtaagcgtgacataactaatttaccatgcagtctgggtcc

SEQ ID NO: 118 - DNA, 54 bp, Artificial Sequence
  AVPR2 TEF F
gaaagcatagcaatctaatctaagttttaattacaaatgctcatggcctctacc

SEQ ID NO: 119 - DNA, 48 bp, Artificial Sequence
  AVPR2 CYC1b R
gtgaatgtaagcgtgacataactaatttaagaagaggtgtctttggcc

SEQ ID NO: 120 - DNA, 55 bp, Artificial Sequence
  CHRM1 TEF F
gaaagcatagcaatctaatctaagttttaattacaaatgaatacaagcgctcctc

SEQ ID NO: 121 - DNA, 48 bp, Artificial Sequence
  CHRM1 CYC1b R
gtgaatgtaagcgtgacataactaatttaacattgtctgctaggtgtt

SEQ ID NO: 122 - DNA, 60 bp, Artificial Sequence
  CHRM3 TEF F
gaaagcatagcaatctaatctaagttttaattacaaatgacgctgcataataattcaacc

SEQ ID NO: 123 - DNA, 44 bp, Artificial Sequence
  CHRM3 CYC1b R
gtgaatgtaagcgtgacataactaatttagagcgcttgttctgg

SEQ ID NO: 124 - DNA, 55 bp, Artificial Sequence
  CHRM5 TEF F
gaaagcatagcaatctaatctaagttttaattacaaatggagggcgactcatatc

SEQ ID NO: 125 - DNA, 47 bp, Artificial Sequence
  CHRM5 CYC1b R
gtgaatgtaagcgtgacataactaatttatggcaatttgctattgcc

SEQ ID NO: 126 - DNA, 54 bp, Artificial Sequence
  CNR2 TEF F
gaaagcatagcaatctaatctaagttttaattacaaatggaagagtgttgggtg

SEQ ID NO: 127 - DNA, 45 bp, Artificial Sequence
  CNR2 CYC1b R
gtgaatgtaagcgtgacataactaatttagcaatcgctgaggtcc

SEQ ID NO: 128 - DNA, 56 bp, Artificial Sequence
  GPR43 TEF F (FFAR2)
gaaagcatagcaatctaatctaagttttaattacaaatgcttccagactggaaatc

SEQ ID NO: 129 - DNA, 49 bp, Artificial Sequence
  GPR43 CYC1b R (FFAR2)
gtgaatgtaagcgtgacataactaatttactcagttgtgaagtcggatg

SEQ ID NO: 130 - DNA, 55 bp, Artificial Sequence
  GPR35 TEF F
gaaagcatagcaatctaatctaagttttaattacaaatgaacggcacgtataaca

SEQ ID NO: 131 - DNA, 45 bp, Artificial Sequence
  GPR35 CYC1b R
gtgaatgtaagcgtgacataactaatttaggccagtgtcacacac

SEQ ID NO: 132 - DNA, 54 bp, Artificial Sequence
  GPR4 TEF F
gaaagcatagcaatctaatctaagttttaattacaaatgggcaaccatacctgg

SEQ ID NO: 133 - DNA, 44 bp, Artificial Sequence
  GPR4 CYC1b R
gtgaatgtaagcgtgacataactaatttactgagctgggggcag

SEQ ID NO: 134 - DNA, 60 bp, Artificial Sequence
  GPR65 TEF F
gaaagcatagcaatctaatctaagttttaattacaaatgaacagtacttgcatcgaagag

SEQ ID NO: 135 - DNA, 47 bp, Artificial Sequence
  GPR65 CYC1b R
gtgaatgtaagcgtgacataactaatttactccaggacttccagctc

SEQ ID NO: 136 - DNA, 54 bp, Artificial Sequence
  GPR68 TEF F
gaaagcatagcaatctaatctaagttttaattacaaatggggaacatcactgcc

SEQ ID NO: 137 - DNA, 46 bp, Artificial Sequence
  GPR68 CYC1b R
gtgaatgtaagcgtgacataactaatttatgcaagtctcccggttg

SEQ ID NO: 138 - DNA, 52 bp, Artificial Sequence
  HCAR2 TEF F
gaaagcatagcaatctaatctaagttttaattacaaatgaaccggcaccacc

SEQ ID NO: 139 - DNA, 43 bp, Artificial Sequence
  HCAR2 CYC1b R
gtgaatgtaagcgtgacataactaatttaggggctcgttgggc

SEQ ID NO: 140 - DNA, 54 bp, Artificial Sequence
  HCAR3 TEF F
gaaagcatagcaatctaatctaagttttaattacaaatgaatcgccaccacctg

SEQ ID NO: 141 - DNA, 48 bp, Artificial Sequence
  HCAR3 CYC1b R
gtgaatgtaagcgtgacataactaatttattcgatacaacagcccaac

SEQ ID NO: 142 - DNA, 58 bp, Artificial Sequence
  HTR4 TEF F
gaaagcatagcaatctaatctaagttttaattacaaatggataagctggatgctaacg

SEQ ID NO: 143 - DNA, 49 bp, Artificial Sequence
  HTR4 CYC1b R
gtgaatgtaagcgtgacataactaatttaagtatctgaaggttgagccg

SEQ ID NO: 144 - DNA, 54 bp, Artificial Sequence
  LPAR1 TEF F
gaaagcatagcaatctaatctaagttttaattacaaatggccgctattagcacc

SEQ ID NO: 145 - DNA, 49 bp, Artificial Sequence
  LPAR1 CYC1b R
gtgaatgtaagcgtgacataactaatttagaccacggaatgatcgttgg

SEQ ID NO: 146 - DNA, 53 bp, Artificial Sequence
  LPAR4 TEF F
gaaagcatagcaatctaatctaagttttaattacaaatgggtgaccggcgatt

SEQ ID NO: 147 - DNA, 50 bp, Artificial Sequence
  LPAR4 CYC1b R
gtgaatgtaagcgtgacataactaatttaaaaggtactttccagcatcag

SEQ ID NO: 148 - DNA, 56 bp, Artificial Sequence
  MRGPRD TEF F
gaaagcatagcaatctaatctaagttttaattacaaatgaaccagacgctgaactc

SEQ ID NO: 149 - DNA, 50 bp, Artificial Sequence
  MRGPRD CYC1b R
gtgaatgtaagcgtgacataactaatttatgctcccatttcgttagttcc

SEQ ID NO: 150 - DNA, 53 bp, Artificial Sequence
  MTNR1A TEF F
gaaagcatagcaatctaatctaagttttaattacaaatgcagggcaacggttc

SEQ ID NO: 151 - DNA, 48 bp, Artificial Sequence
  MTNR1A CYC1b R
gtgaatgtaagcgtgacataactaatttacacactgtcgactttcacg

SEQ ID NO: 152 - DNA, 54 bp, Artificial Sequence
  MTNR1B TEF F
gaaagcatagcaatctaatctaagttttaattacaaatgagcgagaacggttcc

SEQ ID NO: 153 - DNA, 44 bp, Artificial Sequence
  MTNR1B CYC1b R
gtgaatgtaagcgtgacataactaatttacaaagcgtcggcctg

SEQ ID NO: 154 - DNA, 54 bp, Artificial Sequence
  PTAFR TEF F
gaaagcatagcaatctaatctaagttttaattacaaatggagccacatgacagt

SEQ ID NO: 155 - DNA, 49 bp, Artificial Sequence
  PTAFR CYC1b R
gtgaatgtaagcgtgacataactaatttaattcttcagactgttccctg

SEQ ID NO: 156 - DNA, 52 bp, Artificial Sequence
  PTGER3 TEF F
gaaagcatagcaatctaatctaagttttaattacaaatgaaagagacgcgcg

SEQ ID NO: 157 - DNA, 45 bp, Artificial Sequence
  PTGER3 CYC1b R
gtgaatgtaagcgtgacataactaatttaccgttcgagatggtcg

SEQ ID NO: 158 - DNA, 55 bp, Artificial Sequence
  S1PR1 TEF F
gaaagcatagcaatctaatctaagttttaattacaaatgggaccaacatcagtcc

SEQ ID NO: 159 - DNA, 52 bp, Artificial Sequence
  S1PR1 CYC1b R
gtgaatgtaagcgtgacataactaatttagcttgagctattcacattaccac

SEQ ID NO: 160 - DNA, 55 bp, Artificial Sequence
  S1PR2 TEF F
gaaagcatagcaatctaatctaagttttaattacaaatgggctcactttacagtg

SEQ ID NO: 161 - DNA, 44 bp, Artificial Sequence
  S1PR2 CYC1b R
gtgaatgtaagcgtgacataactaatttacacgacggtgttgcc

SEQ ID NO: 162 - DNA, 52 bp, Artificial Sequence
  S1PR3 TEF F
gaaagcatagcaatctaatctaagttttaattacaaatggcaaccgcccttc

SEQ ID NO: 163 - DNA, 53 bp, Artificial Sequence
  S1PR3 CYC1b R
gtgaatgtaagcgtgacataactaatttagttacagaatatgccgttttggag

SEQ ID NO: 164 - DNA, 55 bp, Artificial Sequence
  SSTR5 TEF F
gaaagcatagcaatctaatctaagttttaattacaaatggaaccactcttccctg

SEQ ID NO: 165 - DNA, 48 bp, Artificial Sequence
  SSTR5 CYC1b R
gtgaatgtaagcgtgacataactaatttacagtttgctggtctgcatc

SEQ ID NO: 166 - DNA, 54 bp, Artificial Sequence
  SUCNR1 TEF F
gaaagcatagcaatctaatctaagttttaattacaaatgctgggaattatggcc

SEQ ID NO: 167 - DNA, 50 bp, Artificial Sequence
  SUCNR1 CYC1b R
gtgaatgtaagcgtgacataactaatttacttctccctaaaagacaggag

SEQ ID NO: 168 - DNA, 120 bp, Artificial Sequence
  SST2 del
ttgcacgcactatctgaggcgttataggttcaatttggtaattaaagatagagttgtaagtttcattgagagtcttactcatcttcaggtacaattgcacaaacagtccttttttttttc

SEQ ID NO: 169 - DNA, 21 bp, Artificial Sequence
  X-2 LP Seq R
agggacctagacttcaggttg

SEQ ID NO: 170 - DNA, 20 bp, Artificial Sequence
  ADORA1.qF.2
tgaagatctggaacgaccac

SEQ ID NO: 171 - DNA, 19 bp, Artificial Sequence
  ADORA2A.qF.3
gagtcacgagctgaaaggc

SEQ ID NO: 172 - DNA, 19 bp, Artificial Sequence
  ADORA2B.qF.3
atgtgaagtccggcaatgg

SEQ ID NO: 173 - DNA, 20 bp, Artificial Sequence
  ADRA2A.qF.1
aggcgataggaaacgaatcg

SEQ ID NO: 174 - DNA, 20 bp, Artificial Sequence
  ADRA2B.qF.1
ttcaatcaggatttccggcg

SEQ ID NO: 175 - DNA, 19 bp, Artificial Sequence
  AVPR2.qF.2
gacgaatcctgcactaccg

SEQ ID NO: 176 - DNA, 20 bp, Artificial Sequence
  CHRM1.qF.2
gggtcagttcacagaacacc

SEQ ID NO: 177 - DNA, 20 bp, Artificial Sequence
  CHRM3.qF.2
aaagaaacgacgaaagcagc

SEQ ID NO: 178 - DNA, 20 bp, Artificial Sequence
  CHRM5.qF.1
ggcagggcaatagcaaattg

SEQ ID NO: 179 - DNA, 20 bp, Artificial Sequence
  FFAR2.qF.1
atgccatcatccgacttcac

SEQ ID NO: 180 - DNA, 21 bp, Artificial Sequence
  GPR4.qF.5
caggtgcaattgaagatgctg

SEQ ID NO: 181 - DNA, 20 bp, Artificial Sequence
  GPR65.qF.1
ctgaagttctgtaccggacg

SEQ ID NO: 182 - DNA, 20 bp, Artificial Sequence
  GPR68.qF.1
ttttcaaacccccaactccc

SEQ ID NO: 183 - DNA, 20 bp, Artificial Sequence
  HCAR2.qF.1
ctggtcccctagctatctgg

SEQ ID NO: 184 - DNA, 20 bp, Artificial Sequence
  HCAR3.qF.1
agcttggagaaacagttggg

SEQ ID NO: 185 - DNA, 19 bp, Artificial Sequence
  HTR4.qF.2
agtcccagtgtcatcctcc

SEQ ID NO: 186 - DNA, 20 bp, Artificial Sequence
  LPAR1.qF.3
cagatcggtcagcttcatcc

SEQ ID NO: 187 - DNA, 20 bp, Artificial Sequence
  LPAR4.qF.1
acagaaaccccactgacaac

SEQ ID NO: 188 - DNA, 19 bp, Artificial Sequence
  MRGPRD.qF.5
cccacagtcggaactaacg

SEQ ID NO: 189 - DNA, 20 bp, Artificial Sequence
  MTNR1A.qF.1
tggcagatcgagtcaaatgg

SEQ ID NO: 190 - DNA, 20 bp, Artificial Sequence
  MTNR1B.qF.2
tatccaagacgcatccaagg

SEQ ID NO: 191 - DNA, 20 bp, Artificial Sequence
  PTAFR.qF.1
tagggccactaccgatacag

SEQ ID NO: 192 - DNA, 20 bp, Artificial Sequence
  PTGER3.qF.1
tgtcaaatccgctaccacac

SEQ ID NO: 193 - DNA, 20 bp, Artificial Sequence
  S1PR1.qF.5
accctcagaaagatgaaggc

SEQ ID NO: 194 - DNA, 20 bp, Artificial Sequence
  S1PR3.qF.1
catggacaaaaatgcagccc

SEQ ID NO: 195 - DNA, 20 bp, Artificial Sequence
  SSTR5.qF.1
gaccagacaggattcgacag

SEQ ID NO: 196 - DNA, 20 bp, Artificial Sequence
  SUCNR1.qF.1
gcggcacaattttaagtccc

SEQ ID NO: 197 - DNA, 20 bp, Artificial Sequence
  ZMRUBY3.qF.2
aaacctacgtagtgcaacgc

SEQ ID NO: 198 - DNA, 20 bp, Artificial Sequence
  CNR2.qF.4
attctagggacctggacctc

SEQ ID NO: 199 - DNA, 20 bp, Artificial Sequence
  GPR35.qF.1
tcaagaggcttctgcacttg

SEQ ID NO: 200 - DNA, 20 bp, Artificial Sequence
  S1PR2.qF.6
ttctcccaccttccttgagg

SEQ ID NO: 201 - DNA, 20 bp, Artificial Sequence
  UnTS
ttgcgtaagtggcccctagc